(19) United States
(12) Patent Application Publication
(10) Pub. No.: US 2023/0331792 A1
(43) Pub. Date: Oct. 19, 2023

(54) NEW MULTI-FUNCTIONAL OLIGOPEPTIDES

(71) Applicant: ENLITISA (SHANGHAI) PHARMACEUTICAL CO., LTD., Shanghai (CN)

(72) Inventors: Ming GU, Jiangyin City, Jiangsu Province (CN); Maoqian SONG, Jiangyin City, Jiangsu Province (CN); Jan-Christer JANSON, Uppsala (SE); Bengt Ingemar SAMUELSSON, Solna (SE)

(21) Appl. No.: 17/781,438

(22) PCT Filed: Dec. 2, 2020
(86) PCT No.: PCT/CN2020/133439

§ 371 Date: Jun. 1, 2022

(30) Foreign Application Priority Data
Dec. 2, 2019 (WO) PCT/CN2019/122391
Nov. 6, 2020 (WO) PCT/CN2020/127176

(51) Int. Cl. C07K 14/435; A61P 17/02; C07K 7/06

(52) U.S. Cl. CPC C07K 14/43504; A61P 17/02; C07K 7/06; C07K 7/08

(57) ABSTRACT

There is provided a compound of formula (I), A-Q-B I wherein Q represents a structural fragment of formula (II), wherein: the squiggly lines and m have meanings given in the description, and wherein A and B have meanings given in the description, but may represent a peptide component of the amino acid sequence: [W-Lys-X¹-Ser-U—X²—Y]ₙ—W-Lys-X¹-Ser-U—X²—Y— (SEQ ID No: 3) wherein the dashed line, n, W, X¹, U, X² and Y have meanings given in the description, which compounds are useful in medicine, including as pharmaceutical excipients, adhesives and film-forming materials and/or are useful in the treatment of conditions characterised by inflammation, including wounds, burns, and disorders of the mucosa, such as anorectal diseases, inflammatory bowel diseases, gynaecological diseases and dental diseases.

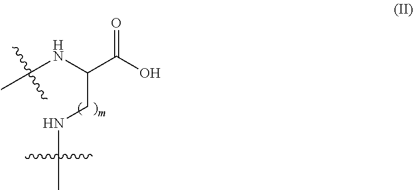

This application is a national stage application under 35 U.S.C. § 371 of International Application No. PCT/CN2020/133439, filed Dec. 2, 2020, which is a continuation-in-part of and claims priority benefit of International Application No. PCT/CN2020/127176, filed Nov. 6, 2020 and International Application No. PCT/CN2019/122391, filed Dec. 2, 2019.

The instant application contains a Sequence Listing which has been submitted electronically in ASCII format and is hereby incorporated by reference in its entirety. Said ASCII copy, created on Dec. 21, 2022, is named 147432_001450.txt and is 45,656 bytes in size.

### FIELD OF THE INVENTION

This invention relates to new peptides, the use of such peptides in human medicine, as pharmaceutically-active ingredients or otherwise, and to pharmaceutical compositions comprising them. In particular, the invention relates to the use of those peptides and compositions in the treatment of various conditions including inflammation.

### BACKGROUND AND PRIOR ART

Inflammation is typically characterized as a localised tissue response to e.g. invasion of microorganisms, certain antigens, damaged cells or physical and/or chemical factors. The inflammatory response is normally a protective mechanism which serves to destroy, dilute or sequester both the injurious agent and the injured tissue, as well as to initiate tissue healing.

Inflammation may result from physical trauma, infection, some chronic diseases (e.g. psoriasis and autoimmune diseases, such as rheumatoid arthritis) and/or chemical and/or physiological reactions to external stimuli (e.g. as part of an allergic response). A complex series of events may be involved, in which inflammatory mediators increase blood flow and dilation of local blood vessels, resulting in redness and heat, the exudation of fluids, often resulting in localised swelling, leukocytic migration into the inflamed area, and pain.

Many conditions/disorders are characterized by, and/or are caused by, abnormal, tissue-damaging inflammation. Such conditions are typically characterized by activation of immune defence mechanisms, resulting in an effect that is more harmful than beneficial to the host, and are generally associated with varying degrees of tissue redness or hyperemia, swelling, hyperthermia, pain, itching, cell death, tissue destruction, cell proliferation and/or loss of function. Examples include inflammatory bowel diseases, rheumatoid arthritis, multiple sclerosis, psoriasis, glomerulonephritis and transplant rejection.

Typically, a complex series of events results in inflammatory changes such as increased blood flow through dilation of local blood vessels, resulting in redness and heat, the extravasation of leukocytes and plasma, often resulting in localised swelling, activation of sensory nerves (resulting in pain in some tissues) and loss of function. These inflammatory changes are triggered by a cascade of cellular and biochemical events involving cells like neutrophils, monocytes, macrophages and lymphocytes together with inflammatory mediators such as vasoactive amines, cytokines, complement factors and reactive oxygen species.

Amongst other things, inflammation plays a key role in the wound healing process. Wounds and burns can therefore be classified as conditions with which inflammation is associated. Traditional thinking in the art is that anti-inflammatory drugs should not be applied directly to open wounds, as this would be detrimental to the progress of wound healing.

Fibrosis is defined by the excessive accumulation of fibrous connective tissue (components of the extracellular matrix (ECM) such as collagen and fibronectin) in and around inflamed or damaged tissue. Although collagen deposition is typically a reversible part of wound healing, it can often evolve into a progressively irreversible fibrotic response if tissue injury is severe, or if the wound-healing response itself becomes dysregulated. Furthermore, fibrogenesis is known to be a major cause of morbidity and mortality in many chronic inflammatory diseases, as well as end-stage liver disease, kidney disease, idiopathic pulmonary fibrosis (IPF) and heart failure. It is also a pathological feature of many chronic autoimmune diseases, such as scleroderma, rheumatoid arthritis, Crohn's disease, ulcerative colitis, myelofibrosis and systemic lupus erythematosus. Fibrosis may also influence the pathogenesis of many progressive myopathies, metastasis and graft rejection.

Mussel adhesive protein (MAP), also known as Mytilus edulis foot protein (mefp), is a protein that is secreted by marine shellfish species, such as Mytilus edulis, Mytilus coruscus and Perna viridis. Eleven identified separate adhesive protein subtypes have been derived from mussels, including the collagens pre-COL-P, pre-COL-D and pre-COL-NG; the mussel feet matrix proteins PTMP (proximal thread matrix protein) and DTMP (distal thread matrix protein); and mfp proteins mfp-2 (sometimes referred to as ‘mefp-2’, hereinafter used interchangeably), mfp-3/mefp-3, mfp-4/mefp-4, mfp-5/mefp-5, mfp-6/mefp-6 and, most preferably mfp-1/mefp-1 (see, for example, Zhu et al., Advances in Marine Science, 2014, 32, 560-568 and Gao et al., Journal of Anhui Agr. Sci., 2011, 39, 19860-19862).

A significant portion of mefp-1 consists of 70 to 90 tandem repeats of the decapeptide: Ala-Lys-Pro-Ser-Tyr-Hyp-Hyp-Thr-DOPA-Lys (SEQ ID No: 1; see Waite, Int. J. Adhesion and Adhesives, 1987, 7, 9-14). This decapeptide sequence may be isolated as a low molecular weight derivative of naturally-occurring MAPs, or may be synthesized, for example as described by Yamamoto in J. Chem. Soc., Perkin Trans., 1987, 1, 613-618. See also Dalsin et al., J. Am. Chem. Soc., 2003, 125, 4253-4258.

Analogues of the decapeptide, notably Ala-Lys-Pro-Ser-Tyr-Hyp-Hyp-Thr-Tyr-Lys (SEQ ID No: 2) have also been disclosed. See, for example, U.S. Pat. No. 5,616,311 and WO 96/39128.

The use of lysine amino acid residues to prepare multi antigen peptides has been disclosed in, for example, Tam Proc. Natl. Acad., Sci. USA, 1988, 85, 5409-5413, Rao et al., J. Am. Chem. Soc., 1994, 116, 6975-6976, U.S. Pat. No. 5,229,490 and WO 2010/038220.

The use of peptide-based scaffolds as drug delivery vehicles has been disclosed. See, for example, Brokx et al, J. Control. Release, 2002, 78, 115-123.

There is a clear need for new and/or improved medicines that may be used in the treatment of inflammation and conditions characterised thereby.

### BRIEF DESCRIPTION OF THE DRAWINGS

FIG. 1 shows unhealed wound rate in an acute wound mouse model.

FIG. 2 shows measured amounts of VEGF in wound tissues in a diabetic wound mouse model.

FIG. 3 shows measured amounts of TGFβ1 in wound tissues in a diabetic wound mouse model.

FIG. 4 shows the effect on swelling caused by acute inflammation in a mouse ear swelling model.

FIGS. 5 and 6 show Evans blue content in rectal and anal tissue indicating vascular permeability of test compounds.

FIGS. 7, 8 and 9 show the effect on body weight, ulcerative surface and general appearance, respectively, in a TNBS-induced ulcerative proctitis model.

FIG. 10 is photographic evidence of the effect of test compound on operative wound healing.

FIG. 11 shows the bioadhesive properties of a compound of the invention.

FIGS. 12 and 13 show plasma concentration versus time curves for mesalazine and montelukast, respectively, when administered along with, and in the absence of, a compound of the invention.

### DISCLOSURE OF THE INVENTION

According to a first aspect of the invention, there is provided a compound of formula I,

A-Q-B I

wherein:
A and B independently represent Z or A¹-Q¹-B¹;
Q represents a structural fragment of formula II,

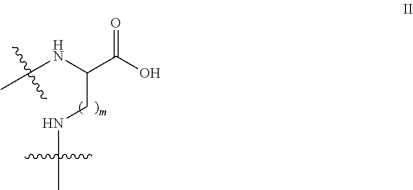

wherein:
the squiggly lines represent points of attachment of Q to A and/or B; and
m represents an integer 1 to 4;
A¹ and B¹ independently represent Z or A²-Q²-B²;
A² and B² independently represent Z or Z-Q³-Z;
Q¹, Q² and Q³ independently represent structural fragments of formula III,

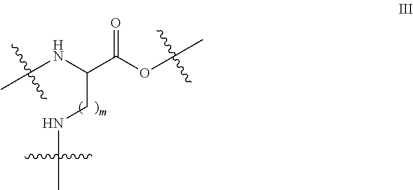

wherein:
the squiggly lines adjacent to the NH groups represent the points of attachment of Q¹, Q² and Q³ to A¹ and/or B¹, A² and/or B², and Z, respectively; and the squiggly line adjacent to the O atom represents the point of attachment of Q¹, Q² and Q³ to Q, Q¹ and Q², respectively; and m is as defined above;
on each occasion that it is employed, Z represents a peptide component of the amino acid sequence:

(SEQ ID NO: 3)
[W-Lys-X¹-Ser-U-X²-Y]ₙ-W-Lys-X¹-Ser-U-X²-Y---

wherein:
the dashed line represents the point of attachment of Z to the rest of the molecule;
n represents 0 or an integer 1 to 4; and,
on each occasion that they are employed:
W represents a 1 or 2 amino acid sequence, in which the amino acids are selected from one or more of the group Lys, Ala, DOPA and a 3,4-dihydrocinnamic acid (HCA) residue, provided that, when present, the HCA residue is located at the N-terminus of the peptide sequence Z;
X¹ represents Pro, Hyp or diHyp;
U represents Tyr or DOPA;
X² represents Ser, Pro, Hyp or diHyp; and
Y represents a 1 to 5 (e.g. a 1 to 4) amino acid sequence, in which the amino acids are selected from one or more of the group Lys, Ala, Pro, Hyp, diHyp, Thr, DOPA and Tyr,
as well as regioisomers, stereoisomers, and pharmaceutically- or cosmetically-acceptable salts of said compound,
which compounds, regioisomers, stereoisomers and salts are referred to together hereinafter as ‘the compounds of the invention’.

Compounds of the invention that may be mentioned include those in which:
W represents a 1 or 2 amino acid sequence, in which the amino acids are selected from one or more of the group Lys, Ala and DOPA;
X¹ represents Pro;
X² represents Ser, Pro or Hyp;
Y represents a 1 to 5 (e.g. a 1 to 4) amino acid sequence, in which the amino acids are selected from one or more of the group Lys, Ala, Pro, Hyp, Thr, DOPA and Tyr.

Preferred compounds of the invention that may be mentioned are those wherein m represents 1, 3 or, more preferably 4, such that one or more of Q, Q¹, Q² and Q³ represent Lys or, more properly, ‘a Lys fragment’, in accordance with what are defined above as ‘the structural fragments of formulae II and III’ (as appropriate).

On each occasion that they are employed, Q, Q¹, Q² and Q³ may each be attached to zero, one or two Z groups.

In this respect, preferred compounds of the invention include those in which:
one of A or B represents Z and the other represents A¹-Q¹-B¹; or, more preferably,
A and B both represent Z, or both represent A¹-Q¹-B¹,
in which, in each case, Q¹ preferably represents a Lys fragment and Z is as hereinbefore defined.

Further preferred compounds of the invention also include those in which: one of A¹ and B¹ represents Z and the other represents A²-Q²-B²; or, more preferably, A¹ and B¹ both represent Z, or both represent A²-Q²-B²,
in which, in each case, Q² preferably represents a Lys fragment, and Z is as hereinbefore defined.

Further preferred compounds of the invention also include those in which:
one of A² and B² represents Z and the other represents Z-Q³-Z; or, more preferably,
A² and B² both represent Z, or both represent or Z-Q³-Z,
in which, in each case, Q³ preferably represents a Lys fragment, and Z is as hereinbefore defined.

More preferred compounds of the invention include those in which A² and B² both represent Z.

Peptide components of compounds of the invention that may be mentioned include those in which n is 0, 1 or 4, or, more preferably, n is 0.

Preferred compounds of the invention include those in which:
X¹ represents Hyp or, more preferably, Pro;
X² represents Hyp;
W represents HCA, HCA-Ala-, preferably Ala or Lys-Ala or, more preferably DOPA or DOPA-Ala-; and/or
Y represents a 5, preferably a 3 or, more preferably, a 4 amino acid sequence, in which the amino acids are selected from one or more of the group Lys, Ala, Hyp, Thr, DOPA and Tyr.

More preferably, compounds of the invention also include those in which Y represents a 4 amino acid sequence selected from the group -Pro-Y¹-Y²-Lys- (SEQ ID NO:86) or, more preferably, -Hyp-Y¹-Y²-Lys- (SEQ ID NO:87) and -Thr-Y¹-Y²-Lys (SEQ ID NO:88), wherein Y¹ and Y² are each independently selected from the group Pro or, more preferably, Ala, Hyp, Thr, DOPA and Tyr.

Further preferred compounds of the invention include those in which the amino acid sequence defined b Y is selected from the group:

(SEQ ID NO: 89)
-Pro-Thr-DOPA-Lys-;
(SEQ ID NO: 90)
-Pro-Thr-Tyr-Lys-;
(SEQ ID NO: 91)
-Thr-Tyr-Pro-Lys-;
and
(SEQ ID NO: 92)
-Thr-DOPA-Pro-Lys-;
and, more preferably,
(SEQ ID NO: 93)
-Hyp-Thr-Tyr-Lys-;
(SEQ ID NO: 94)
-Hyp-Thr-DOPA-Lys-;
(SEQ ID NO: 95)
-Hyp-Thr-Ala-Lys-;
(SEQ ID NO: 96)
-Thr-Tyr-Hyp-Lys-;
(SEQ ID NO: 97)
-Thr-DOPA-Hyp-Lys-;
and
(SEQ ID NO: 98)
-Thr-Ala-Hyp-Lys-.

When Y represents a 2 amino acid sequence, preferred compounds of the invention include those in which the amino acid sequence defined by Y is selected from the group -Hyp-Thr-, -Thr-Tyr-, -Pro-Thr- and -Thr-DOPA-.

Other preferred compounds of the invention that may be mentioned include those in which the amino acid sequence defined by Y is selected from the group -Thr-Tyr-Lys-, -Tyr-Pro-Lys-, -DOPA-Pro-Lys-, -Hyp-Thr-Tyr-, -Hyp-Thr-Tyr-Hyp-Lys- (SEQ ID NO: 99) and, more preferably, the groups -Thr-Tyr-Hyp-Lys-DOPA- (SEQ ID NO: 100) and -Hyp-Thr-DOPA-.

Compounds of the invention that may be mentioned include those in which:
U represents Tyr; and/or
W represents Ala.

In this respect, further compounds of the invention that may be mentioned include those wherein Z is selected from the group:

(SEQ ID No: 2)
Ala-Lys-Pro-Ser-Tyr-Hyp-Hyp-Thr-Tyr-Lys---;
(SEQ ID No: 1)
Ala-Lys-Pro-Ser-Tyr-Hyp-Hyp-Thr-DOPA-Lys---;
(SEQ ID No: 4)
Ala-Lys-Pro-Ser-Tyr-Hyp-Thr-Tyr-Hyp-Lys---;
(SEQ ID No: 5)
Ala-Lys-Pro-Ser-Tyr-Hyp-Thr-DOPA-Hyp-Lys---;
(SEQ ID No: 6)
Ala-Lys-Pro-Ser-Tyr-Hyp-Thr-Tyr-Hyp-Lys-DOPA---;
(SEQ ID No: 7)
Ala-Lys-Pro-Ser-Tyr-Ser-Hyp-Thr-Tyr-Lys-Ala-Lys-
Pro-Ser-Tyr-Ser-Hyp-Thr-Tyr-Lys--;
and
(SEQ ID NO: 8)
Ala-Lys-Pro-Ser-Tyr-Hyp-Hyp-Thr-Tyr-Lys-Ala-Lys-
Pro-Ser-Tyr-Hyp-Hyp-Thr-Tyr-Lys-Ala-Lys-Pro-Ser-
Tyr-Hyp-Hyp-Thr-Tyr-Lys-Ala-Lys-Pro-Ser-Tyr-Hyp-
Hyp-Thr-Tyr-Lys-Ala-Lys-Pro-Ser-Tyr-Hyp-Hyp-Thr-
Tyr-Lys---.

Compounds of the invention that may be mentioned include those in which:
U represents Tyr; and/or
W represents Lys-Ala-.

In this respect, further compounds of the invention that may be mentioned include those wherein Z is selected from the group:

(SEQ ID No: 9)
Lys-Ala-Lys-Pro-Ser-Tyr-Hyp-Hyp-Thr-Tyr---;
(SEQ ID No: 10)
Lys-Ala-Lys-Hyp-Ser-Tyr-Hyp-Hyp-Thr-DOPA---;
and, more preferably,
(SEQ ID NO: 11)
Lys-Ala-Lys-Pro-Ser-Tyr-Hyp-Hyp-Thr-DOPA---.

Further compounds of the invention that may be mentioned include those in which:
U represents Tyr; and/or
W represents HCA, HCA-Ala- or, more preferably, DOPA or DOPA-Ala-.

In this respect, compounds of the invention that may be mentioned include those wherein Z is selected from the group:

(SEQ ID No: 12)
HCA-Ala-Lys-Pro-Ser-Tyr-Hyp-Thr-Tyr-Hyp-Lys---;
(SEQ ID No: 13)
HCA-Ala-Lys-Pro-Ser-Tyr-Hyp-Hyp-Thr-Tyr-Lys---;
(SEQ ID No: 14)
HCA-Lys-Pro-Ser-Tyr-Hyp-Hyp-Thr-Ala-Lys---;
(SEQ ID No: 15)
HCA-Lys-Pro-Ser-Tyr-Hyp-Thr-Ala-Hyp-Lys---;

and, more preferably, wherein Z is selected from the group:

(SEQ ID No: 16)
DOPA-Lys-Pro-Ser-Tyr-Hyp-Thr-Ala-Hyp-Lys---;
(SEQ ID No: 17)
DOPA-Lys-Pro-Ser-Tyr-Hyp-Hyp-Thr-Ala-Lys---;
(SEQ ID No: 18)
DOPA-Ala-Lys-Pro-Ser-Tyr-Hyp-Thr-Tyr-Hyp-Lys---;
and
(SEQ ID NO: 19)
DOPA-Ala-Lys-Pro-Ser-Tyr-Hyp-Hyp-Thr-Tyr-Lys---.

Other compounds of the invention that may be mentioned include those in which U represents DOPA.

In this respect, compounds of the invention that may be mentioned include those wherein Z is selected from the group:

(SEQ ID No: 20)
Ala-Lys-Pro-Ser-DOPA-Hyp-Thr-DOPA-Hyp-Lys---;
(SEQ ID No: 21)
Ala-Lys-Pro-Ser-DOPA-Hyp-Hyp-Thr-DOPA-Lys---;
(SEQ ID No: 22)
Lys-Ala-Lys-Pro-Ser-DOPA-Hyp-Hyp-Thr-DOPA---;
(SEQ ID No: 23)
Lys-Ala-Lys-Hyp-Ser-DOPA-Hyp-Hyp-Thr-DOPA---;

and, more preferably, wherein Z is selected from the group:
Ala-Lys-Pro-Ser-DOPA-Hyp-Hyp-Thr-DOPA-Lys-Ala-lys-Pro-Ser-DOPA-Hyp-Hyp-Thr-DOPA-Lys- (SEQ ID No: 24); and
Ala-Lys-Pro-Ser-DOPA-Hyp-Hyp-Thr-DOPA-Lys-Ala-Lys-Pro-Ser-DOPA-Hyp-Hyp-Thr-DOPA-Lys-Ala-Lys-Pro-Ser-DOPA-Hyp-Hyp-Thr-DOPA-Lys-Ala-Lys-Pro-Ser-DOPA-Hyp-Hyp-Thr-DOPA-Lys-Ala-Lys-Pro-Ser-DOPA-Hyp-Hyp-Thr-DOPA-Lys- (SEQ ID No: 25).

Further compounds of the invention that may be mentioned include those in which:
U represents DOPA; and/or
W represents HCA, HCA-Ala- or, more preferably, DOPA or DOPA-Ala-.

Accordingly, particular compounds of the invention that may be mentioned include those wherein Z is selected from the group:

(SEQ ID No: 26)
HCA-Ala-Lys-Pro-Ser-DOPA-Hyp-Thr-DOPA-Hyp-Lys---;
(SEQ ID No: 27)
HCA-Ala-Lys-Pro-Ser-DOPA-Hyp-Hyp-Thr-DOPA-Lys---;
(SEQ ID No: 28)
HCA-Lys-Pro-Ser-DOPA-Hyp-Thr-Ala-Hyp-Lys---;
(SEQ ID No: 29)
HCA-Lys-Pro-Ser-DOPA-Hyp-Hyp-Thr-Ala-Lys---;

and, more preferably, wherein Z is selected from the group:

(SEQ ID No: 30)
DOPA-Lys-Pro-Ser-DOPA-Hyp-Thr-Ala-Hyp-Lys---;
(SEQ ID No: 31)
DOPA-Lys-Pro-Ser-DOPA-Hyp-Hyp-Thr-Ala-Lys---;
(SEQ ID No: 32)
DOPA-Ala-Lys-Pro-Ser-DOPA-Hyp-Thr-DOPA-Hyp-Lys---;
(SEQ ID No: 33)
DOPA-Ala-Lys-Pro-Ser-DOPA-Hyp-Hyp-Thr-Tyr-Lys---;
and
(SEQ ID NO: 34)
DOPA-Ala-Lys-Pro-Ser-DOPA-Hyp-Hyp-Thr-DOPA-Lys---.

Compounds of the invention that may be mentioned include those in which:
A and B both represent Z;
one, or preferably both, Z groups represent:

(SEQ ID No: 4)
Ala-Lys-Pro-Ser-Tyr-Hyp-Thr-Tyr-Hyp-Lys---,
(SEQ ID No: 5)
Ala-Lys-Pro-Ser-Tyr-Hyp-Thr-DOPA-Hyp-Lys---,
(SEQ ID No: 12)
HCA-Ala-Lys-Pro-Ser-Tyr-Hyp-Thr-Tyr-Hyp-Lys---,
(SEQ ID No: 15)
HCA-Lys-Pro-Ser-Tyr-Hyp-Thr-Ala-Hyp-Lys---,
(SEQ ID No: 18)
DOPA-Ala-Lys-Pro-Ser-Tyr-Hyp-Thr-Tyr-Hyp-Lys--,
(SEQ ID No: 20)
Ala-Lys-Pro-Ser-DOPA-Hyp-Thr-DOPA-Hyp-Lys---,
(SEQ ID No: 22)
Lys-Ala-Lys-Pro-Ser-DOPA-Hyp-Hyp-Thr-DOPA---,
(SEQ ID No: 26)
HCA-Ala-Lys-Pro-Ser-DOPA-Hyp-Thr-DOPA-Hyp-Lys---,
(SEQ ID No: 31)
DOPA-Lys-Pro-Ser-DOPA-Hyp-Hyp-Thr-Ala-Lys---,
(SEQ ID No: 32)
DOPA-Ala-Lys-Pro-Ser-DOPA-Hyp-Thr-DOPA-Hyp-Lys---,

or, more preferably, one, or preferably both, Z groups represent:

(SEQ ID No: 4)
Ala-Lys-Pro-Ser-Tyr-Hyp-Thr-Tyr-Hyp-Lys---,
or
(SEQ ID No: 5)
Ala-Lys-Pro-Ser-Tyr-Hyp-Thr-DOPA-Hyp-Lys---;

or, even more preferably, one, or preferably both, Z groups represent:

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---,

and
Q represents a Lys fragment.

Further compounds of the invention that may be mentioned include those in which:
A and B both represent A¹-Q¹-B¹;
A¹ and B¹ both represent Z;
one, or preferably both, Z groups represent:

(SEQ ID No: 4)
Ala-Lys-Pro-Ser-Tyr-Hyp-Thr-Tyr-Hyp-Lys---,
(SEQ ID No: 5)
Ala-Lys-Pro-Ser-Tyr-Hyp-Thr-DOPA-Hyp-Lys---,
(SEQ ID No: 11)
Lys-Ala-Lys-Pro-Ser-Tyr-Hyp-Hyp-Thr-DOPA---,
(SEQ ID No: 12)
HCA-Ala-Lys-Pro-Ser-Tyr-Hyp-Thr-Tyr-Hyp-Lys---,
(SEQ ID No: 13)
HCA-Ala-Lys-Pro-Ser-Tyr-Hyp-Hyp-Thr-Tyr-Lys---,
(SEQ ID No: 18)
DOPA-Ala-Lys-Pro-Ser-Tyr-Hyp-Thr-Tyr-Hyp-Lys---,
(SEQ ID No: 20)
Ala-Lys-Pro-Ser-DOPA-Hyp-Thr-DOPA-Hyp-Lys---,
(SEQ ID No: 21)
Ala-Lys-Pro-Ser-DOPA-Hyp-Hyp-Thr-DOPA-Lys---,
(SEQ ID No: 10)
Lys-Ala-Lys-Hyp-Ser-Tyr-Hyp-Hyp-Thr-DOPA---,
(SEQ ID No: 28)
HCA-Lys-Pro-Ser-DOPA-Hyp-Thr-Ala-Hyp-Lys---,
(SEQ ID No: 30)
DOPA-Lys-Pro-Ser-DOPA-Hyp-Thr-Ala-Hyp-Lys---,
(SEQ ID No: 32)
DOPA-Ala-Lys-Pro-Ser-DOPA-Hyp-Thr-DOPA-Hyp-Lys---,

or, more preferably, one, or preferably both, Z groups represent:

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---;

and
Q¹ represents a Lys fragment.

Further compounds of the invention that may be mentioned include those in which:
A and B both represent A¹-Q¹-B¹;
A¹ and B¹ both represent A²-Q²-B²;
A² and B² both represent Z;
one, or preferably both, Z groups represent:

(SEQ ID No: 4)
Ala-Lys-Pro-Ser-Tyr-Hyp-Thr-Tyr-Hyp-Lys---,
(SEQ ID No: 5)
Ala-Lys-Pro-Ser-Tyr-Hyp-Thr-DOPA-Hyp-Lys---,
(SEQ ID No: 12)
HCA-Ala-Lys-Pro-Ser-Tyr-Hyp-Thr-Tyr-Hyp-Lys---,
(SEQ ID No: 18)
DOPA-Ala-Lys-Pro-Ser-Tyr-Hyp-Thr-Tyr-Hyp-Lys---,
(SEQ ID No: 21)
Ala-Lys-Pro-Ser-DOPA-Hyp-Hyp-Thr-DOPA-Lys---,
(SEQ ID No: 32)
DOPA-Ala-Lys-Pro-Ser-DOPA-Hyp-Thr-DOPA-Hyp-Lys---,

or, more preferably, one, or preferably both, Z groups represent:
DOPA-Ala-Lys-Pro-Ser-Tyr-Hyp-Hyp-Thr-Tyr-Lys- (SEQ ID No: 19),
or, even more preferably, one, or preferably both, Z groups represent:

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---;

and
Q¹ and Q² both represent Lys fragments.

Further compounds of the invention that may be mentioned include those in which:
A and B both represent A¹-Q¹-B¹;
A¹ and B¹ both represent A²-Q²-B²;
A² and B² both represent Z-Q³-Z;
one, or preferably both, Z groups represent:
   and

(SEQ ID No: 2)
Ala-Lys-Pro-Ser-Tyr-Hyp-Hyp-Thr-Tyr-Lys---,
or
(SEQ ID No: 1)
Ala-Lys-Pro-Ser-Tyr-Hyp-Hyp-Thr-DOPA-Lys---;

Q¹, Q² and Q³ all represent Lys fragments.

In further aspect of the invention, there is provided an (isolated) peptide compound of the amino acid sequence:
[Ala-Lys-X¹-Ser-U—X²—Y]p-Ala-Lys-X¹-Ser-U—X¹—Y-G (SEQ ID No: 35)
   wherein
p represents an integer 1 to 4;
G may be absent (in which case Y is the C-terminal amino acid) or G may represent DOPA or dopamine (or, more properly, ‘a dopamine fragment’); and
X¹, U, X² and Y are as hereinbefore defined,
as well as regioisomers, stereoisomers, and pharmaceutically- or cosmetically-acceptable salts of said compound,
which compounds, regioisomers, stereoisomers and salts are referred to hereinafter ‘the linear long-chain compounds of the invention’.

As used herein, the terms dopamine and dopamine fragment refer to a structural fragment of formula IV,

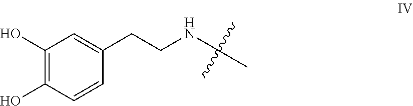 [SMILES: *NCCc1ccc(O)c(O)c1]

wherein the squiggly line represents the point of attachment to Y.

Preferred values of p in linear long-chain compounds of the invention are, in ascending order of preference 2, 3, 1 and 4.

Preferred values of U, X and Y mentioned hereinbefore for compounds of the invention are also preferred for linear long-chain compounds of the invention.

Particular linear long-chain compounds of the invention that may be mentioned are those where G is absent.

In this respect, particular linear long-chain peptide compounds include those of the amino acid sequence:

(SEQ ID No: 36)
Ala-Lys-Pro-Ser-Tyr-Ser-Hyp-Thr-Tyr-Lys-Ala-Lys-
Pro-Ser-Tyr-Ser-Hyp-Thr-Tyr-Lys;
(SEQ ID No: 37)
Ala-Lys-Pro-Ser-Tyr-Hyp-Hyp-Thr-Tyr-Lys-Ala-Lys-
Pro-Ser-Tyr-Hyp-Hyp-Thr-Tyr-Lys-Ala-Lys-Pro-Ser-
Tyr-Hyp-Hyp-Thr-Tyr-Lys-Ala-Lys-Pro-Ser-Tyr-Hyp-
Hyp-Thr-Tyr-Lys-Ala-Lys-Pro-Ser-Tyr-Hyp-Hyp-Thr-
Tyr-Lys;
(SEQ ID No: 38)
Ala-Lys-Pro-Ser-DOPA-Hyp-Hyp-Thr-DOPA-Lys-Ala-Lys-
Pro-Ser-DOPA-Hyp-Hyp-Thr-DOPA-Lys;
(SEQ ID NO: 39)
Ala-Lys-Pro-Ser-DOPA-Hyp-Hyp-Thr-DOPA-Lys-Ala-Lys-
Pro-Ser-DOPA-Hyp-Hyp-Thr-DOPA-Lys-Ala-Lys-Pro-Ser-
DOPA-Hyp-Hyp-Thr-DOPA-Lys-Ala-Lys-Pro-Ser-DOPA-
Hyp-Hyp-Thr-DOPA-Lys-Ala-Lys-Pro-Ser-DOPA-Hyp-Hyp-
Thr-DOPA-Lys;
(SEQ ID No: 40)
Ala-Lys-Pro-Ser-Tyr-Hyp-Hyp-Thr-DOPA-Lys-Ala-Lys-
Pro-Ser-Tyr-Hyp-Hyp-Thr-DOPA-Lys;
(SEQ ID No: 41)
Ala-Lys-Pro-Ser-Tyr-Hyp-Hyp-Thr-DOPA-Lys-Ala-Lys-
Pro-Ser-Tyr-Hyp-Hyp-Thr-DOPA-Lys-Ala-Lys-Pro-Ser-
Tyr-Hyp-Hyp-Thr-DOPA-Lys;
(SEQ ID No: 42)
Ala-Lys-Pro-Ser-Tyr-Hyp-Hyp-Thr-DOPA-Lys-Ala-Lys-
Pro-Ser-Tyr-Hyp-Hyp-Thr-DOPA-Lys-Ala-Lys-Pro-Ser-
Tyr-Hyp-Hyp-Thr-DOPA-Lys-Ala-Lys-Pro-Ser-Tyr-Hyp-
Hyp-Thr-DOPA-Lys;
and
(SEQ ID NO: 43)
Ala-Lys-Pro-Ser-Tyr-Hyp-Hyp-Thr-DOPA-Lys-Ala-Lys-
Pro-Ser-Tyr-Hyp-Hyp-Thr-DOPA-Lys-Ala-Lys-Pro-Ser-
Tyr-Hyp-Hyp-Thr-DOPA-Lys-Ala-Lys-Pro-Ser-Tyr-Hyp-
Hyp-Thr-DOPA-Lys-Ala-Lys-Pro-Ser-Tyr-Hyp-Hyp-Thr-
DOPA-Lys.

For the avoidance of doubt, compounds of the invention as hereinbefore defined, whether a compound of formula I, or a linear long-chain peptide compound of SEQ ID No: 35, are referred to together hereinafter as ‘compounds of the invention’.

As used herein, Pro represents proline, Ala represents alanine, Ser represents serine, Tyr represents tyrosine, Hyp represents hydroxyproline (including 3-hydroxyproline (3Hyp) and 4-hydroxyproline (4Hyp)), diHyp represents dihydroxyproline (including 3,4-dihydroxyproline (3,4diHyp), 3,5-dihydroxyproline (3,5diHyp) and 4,5-dihydroxyproline (4,5diHyp)), Thr represents threonine, Lys represents lysine, Ala represents alanine, DOPA represents 3,4-dihydroxyphenylalanine, Orn represents ornithine and Dap represents diaminopropionic acid. 3,4-Dihydrocinnamic acid (HCA) residues are essentially DOPA residues but without the —NH₂ group in the 2- or α-carbon position relative to the carboxylic acid that is attached to the N-terminal amino acid (whether Lys or Ala).

Compounds of the invention, whether in the form of salts or otherwise, include regioisomers within amino acids of the peptides (for example diHyp, Hyp and Tyr moieties), as well as mixtures of such regioisomers. For example, included within the definition of Tyr are, not only tyrosine (4-hydroxyphenylalanine), but also 2- and 3-hydroxyphenylalanine. Included within the definition of Hyp are 4-hydroxyproline (4Hyp), 3-hydroxyproline (3Hyp) and 5-hydroxyproline (5Hyp). It is more preferred that Hyp residues are 4-hydroxyproline. Similarly, included within the definition of diHyp are 3,4-dihydroxyproline (3,4diHyp), 3,5-dihydroxyproline (3,5diHyp) and 4,5-dihydroxyproline (4,5diHyp). It is more preferred that diHyp residues are 3,4-dihydroxyproline (3,4diHyp).

Also, in addition to the standard central carbon atom of the amino acids in the compounds of the invention (which are normally but not exclusively in the L-configuration), certain amino acids in the sequence comprise further chiral carbon atoms. All such stereoisomers and mixtures (including racemic mixtures) thereof are included within the scope of the invention. In respect, included within the definition of Hyp are trans-4-hydroxy-L-proline, cis-4-hydroxy-L-proline, trans-3-hydroxy-L-proline, cis-3-hydroxy-L-proline, trans-5-hydroxy-L-proline and cis-5-hydroxy-L-proline, however we prefer that the Hyp that is employed in compounds of the invention is 4-hydroxy-L-proline. Similarly, corresponding definitions may be applied to diHyp, in which the two hydroxy groups can also be cis or trans relative to each other. In any event, individual enantiomers of compounds of formula I (and the isolated peptide sequences of SEQ IDs Nos: 4 to 26) that may form part of a compound of the invention are included within the scope of the invention.

Compounds of the invention may be in the form of salts. Salts that may be mentioned include pharmaceutically-acceptable and/or cosmetically-acceptable salts, such as pharmaceutically- and/or cosmetically-acceptable acid addition salts and base addition salts. Such salts may be formed by conventional means, for example by reaction of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of the compound of the invention in the form of a salt with another counter-ion, for example using a suitable ion exchange resin.

Preferred salts include, for example, acetate, hydrochloride, bisulfate, maleate, mesylate, tosylate, alkaline earth metal salts, such as calcium and magnesium, or alkali metal salts, such as sodium and potassium salts. Most preferably, compounds of the invention may be in the form of acetate salts.

Compounds of the invention may be prepared by way of conventional techniques, for example by way of standard amino acid coupling techniques, using standard coupling reagents and solvents, for example as described hereinafter. Compounds of the invention may be synthesised from available starting materials using appropriate reagents and reaction conditions. In this respect, the skilled person may refer to inter alia “Comprehensive Organic Synthesis” by B. M. Trost and I. Fleming, Pergamon Press, 1991. Further references that may be employed include “Heterocyclic Chemistry” by J. A. Joule, K. Mills and G. F. Smith, 3^{rd} edition, published by Chapman & Hall, “Comprehensive Heterocyclic Chemistry II” by A. R. Katritzky, C. W. Rees and E. F. V. Scriven, Pergamon Press, 1996 and “Science of Synthesis”, Volumes 9-17 (Hetarenes and Related Ring Systems), Georg Thieme Verlag, 2006.

Compounds of the invention may be isolated from their reaction mixtures and, if necessary, purified using conventional techniques as known to those skilled in the art. Thus, processes for preparation of compounds of the invention as described herein may include, as a final step, isolation and optionally purification of the compound of the invention.

It will be appreciated by those skilled in the art that, in the processes described above and hereinafter, the functional groups of intermediate compounds may need to be protected by protecting groups. The protection and deprotection of functional groups may take place before or after a reaction.

Protecting groups may be applied and removed in accordance with techniques that are well-known to those skilled in the art and as described hereinafter. For example, protected compounds/intermediates described herein may be converted chemically to unprotected compounds using standard deprotection techniques. The type of chemistry involved will dictate the need, and type, of protecting groups as well as the sequence for accomplishing the synthesis. The use of protecting groups is fully described in ‘Protective Groups in Organic Synthesis’, 5th edition, T. W. Greene & P. G. M. Wutz, Wiley-Interscience (2014), the contents of which are incorporated herein by reference.

Compounds of the invention are useful as human and animal medicine. They are therefore indicated as pharmaceuticals (and/or in veterinary science), although they may also be used as cosmetics and/or as part of a medical device.

Compounds of the invention (and isolated peptide sequences) may also possess pharmacological activity as such, certain pharmaceutically-acceptable (e.g. ‘protected’) derivatives of compounds of the invention may exist or may be prepared which may not possess such activity, but which may be administered and thereafter be metabolised or chemically transformed to form compounds of the invention. Such compounds (which may possess some pharmacological activity, provided that such activity is appreciably lower than that of the active compounds to which they are metabolised/transformed) may therefore be described as ‘prodrugs’ of compounds of the invention.

As used herein, references to prodrugs will include compounds that form a compound of the invention, in an experimentally-detectable amount, within a predetermined time, following administration. All prodrugs of the compounds of the invention are included within the scope of the invention.

When compounds of the invention possess pharmacological activity, they are particularly useful in the treatment of inflammation.

The term ‘treatment of inflammation’ includes the treatment of inflammation in any organ of the body (including soft tissue, joints, nerves, the vascular system, internal organs, especially mucosal surfaces, and particularly the skin), irrespective of the cause, and also includes all such inflammatory disorders or conditions, and/or disorders or conditions characterized by inflammation (e.g. as a symptom).

Inflammatory disorders and/or conditions may be (and are typically) characterized by activation of immune defence mechanisms, resulting in an effect that is more harmful than beneficial to the host. Such conditions are generally associated with varying degrees of tissue redness or hyperemia, swelling, edema, hyperthermia, pain (including aching), exudation of body fluids, itching (pruritis), cell death and tissue destruction, cell proliferation, and/or loss of function.

Inflammatory conditions that may be mentioned include arteritis, diabetes mellitus, metabolic syndrome, rosacea, asthma and allergy, ankylosing spondylitis, chronic obstructive pulmonary disease, gouty arthritis, inflammatory bowel disease (such as Crohn's disease and ulcerative colitis), multiple sclerosis, osteoarthritis, pancreatitis, prostatitis, psoriatic arthritis, rheumatoid arthritis, tendinitis, bursitis, Sjögren's syndrome, systemic lupus erythematosus, uveitis, urticaria, vasculitis, mastocytosis, diabetic vascular complications, migraine, atherosclerosis and associated cardiovascular disorders. A disease state characterised by inflammation that may be mentioned is chronic obstructive pulmonary disease (COPD). A further disease state characterised by inflammation that may be mentioned is inflammatory bowel diseases including Crohn's disease and, especially, ulcerative colitis. Other disease states characterized by inflammation that may be mentioned are gynaecological diseases, such as cervicitis, vaginitis (e.g. radiation vaginitis) and colpitis. Diseases that affect the gastrointestinal tract, such as gastrohelcosis (e.g. gastritis, gastric ulcer, gastric cancer and other stomach mucosa diseases) as well as gastroesophageal reflux disease (GERD), constipation, and gastritis, inflammation associated with cancers and infections (e.g. viral infections, such as the common cold or influenza).

Inflammatory conditions that may be more especially mentioned include inflammations of the skin or mucosa (including the oral, nasal, ocular, vaginal, cervical and/or anorectal mucosae, more particularly the oral or nasal mucosae), such as inflammation resulting from infections (such as viral and/or bacterial infections), or allergic/atopic conditions (such as rhinitis (e.g. allergic rhinitis), pharyngitis, periodontitis, gingivitis, xerophthalmia, conjunctivitis (e.g. allergic conjunctivitis), dermatitis, urticaria (hives) and food allergy); and other inflammatory conditions, such as herpes, drug eruptions, polymorphous light eruptions, sunburn, early manifestations of skin cancers (erythema-like skin lesions), pathological hair loss (including following skin grafting), chemo rash, psoriasis, erythema multiforme, folliculitis, eczema and external otitis. A disease state that may be mentioned is polymorphous light eruptions.

More particularly, compounds may be used to treat certain conditions characterized by inflammation, and/or with which inflammation is associated. Such conditions may include wounds (including abrasions (scratches), incisions (including operative incisions), lacerations, punctures, avulsions, bruising and scarring), and burns (including inflammation resulting from surgery following burns, such as skin grafting) and other conditions, such as hemorrhoids. Wounds may be acute or chronic, and/or may result from one or more inflammatory disorders as defined herein.

Wounds of the skin or mucosa may arise from internal or external physical injury to the membrane surface, or may be caused by (i.e. be a symptom of) an underlying physiological disorder.

Physical (e.g. ‘open’) wounds may be caused by sharp objects (cuts, incisions, punctures) or blunt objects/mechanical forces (lacerations, abrasions, avulsions), physical blows (bruises), heat or chemicals (burns and blisters), UV light (sunburn), cold (chilblains or frostbite). Wounds may be superficial (damage only to the epidermis and/or dermis) or may be full thickness wounds (damage below the epidermis and/or dermis). In serious cases, subcutaneous and/or submucosal tissues, such as muscles, bones, joints, and even internal organs, may be damaged.

Compounds of the invention may be used to relieve the pain (including aching) associated with inflammation and/or wounding. In particular, compounds of the invention may be used to relieve procedural pain and/or non-procedural pain. The skilled person will understand that the term ‘procedural pain’ (i.e. operation pain) refers to acute pain that is associated with medical investigations and treatments conducted for the purpose of healthcare. The term ‘non-procedural’ refers to general pain that is associated with inflammation and/or wounding (e.g. pain associated with dental ulcers, burns and/or scars), and is not a consequence of a particular medical intervention.

Compounds of the invention may be used to treat not only the inflammation, pain (including aching) and/or pruritis (itching) associated with the wound itself and the healing process, but also to prevent the exudation of body fluids from wounds, the risk of infection, and the prevention of physiological reactions that result from inflammation and/or wound healing processes, such as scarring and melanin pigmentation.

Scarring is a consequence of inflammation and/or wound healing and is a general term for the formation of fibrotic tissue that is a consequence of such inflammation/healing.

Compounds of the invention may also be useful in the suppression of the production of melanin pigmentation, which may or may not result from inflammation and/or wound healing. Compounds of the invention may also be useful in the suppression of disorders associated with melanin pigmentation, such as chloasma, freckles, melanosis, malar rash and other chromatosis, skin cancers with melanoma, and chromatosis that is caused by exposure to the sun or skin diseases like acne.

Wounds may also arise as a consequence of (e.g. inflammatory) diseases or disorders. Such wounds may include blistering and/or ulcers of the skin and mucosa. These are common conditions that are often long-lasting and difficult to treat. Skin tissues can often be damaged, removed, liquefied, infected and/or necrotic. Ulcers can lead to secondary consequences to health particularly if they become infected, are hard to heal and are costly to treat. They can also cause significant psychological stress and economic loss to patients, affecting both general well-being and quality of life.

In the alternative, inflammatory skin conditions or diseases in which compounds of the invention find particular utility include psoriasis, acne, eczema and dermatitis, especially allergic/atopic dermatitis, as well as in the treatment of mucosal inflammation as characterized by rhinitis, especially allergic rhinitis, hemorrhoids, chronic obstructive pulmonary disease and ulcerative colitis, for example.

Psoriasis is a chronic, inflammatory skin disease with a tendency to recur (some patients never heal during their entire life). Clinical manifestations of psoriasis mainly include erythema and scales. It can occur over the whole body, but is more commonly observed on the scalp and limbs.

Acne is a follicular (pilosebaceous unit) chronic, inflammatory skin disease, the occurrence of which is closely related to main factors like hypersteatosis, blocked pilosebaceous ducts (including closed and open comedones), bacterial infection and inflammatory reactions, that tends to occur during youth, characterized by multiform skin lesions on the face. The term acne thus includes regular acne and acne rosacea (i.e. copper nose).

Eczema is a skin inflammatory reaction with strong itching caused by a variety of internal and external factors. It has three phases, acute, sub-acute, and chronic. In the acute phase, there is a tendency for the production of exudates, while the chronic phase includes infiltration and hypertrophy. Skin lesions are often itchy and recur easily.

Dermatitis is a common skin disease characterized by coarseness, redness, itching, eczema, and dryness. Small lumps, refractory ulcers, and pigmented spots caused by dermatitis may, if not treated promptly, develop to basal cell carcinoma, squamous cell carcinoma, and malignant melanoma. Dermatitis may be caused by various internal and external infectious or non-infectious factors, including substances (contact dermatitis) or allergy (allergic/atopic dermatitis). Also included is seborrheic dermatitis (seborrheic eczema) and all forms of steroid-dependent dermatitis (including light-sensitive seborrheic, perioral dermatitis, rosacea-like dermatitis, steroid-rosacea, steroid-induced rosacea, iatrosacea, steroid dermatitis resembling rosacea, topical corticosteroid-induced rosacea-like dermatitis and, more particularly, facial corticosteroid addictive dermatitis (FCAD) or facial corticosteroid-dependent dermatitis (FCDD), as characterized by flushing, erythema, telangiectasia, atrophy, papules and/or pustules in the facial area after long-term treatment with (including uncontrolled use, abuse or misuse of) topical corticosteroids; see, for example, Xiao et al., J. Dermatol., 2015, 42, 697-702 and Lu et al., Clin. Exp. Dermatol., 2009, 35, 618-621).

Rhinitis is irritation and inflammation of the mucous membrane inside the nose. Common symptoms of rhinitis include a stuffy nose, runny nose, sneezing and post-nasal drip. The most common kind of rhinitis is allergic rhinitis, caused by an allergen, such as pollen, dust, mould, or flakes of skin from certain animals. It has been surprisingly found that patients with allergic rhinitis who were treated with compounds of the invention experienced relief of eye itchiness, even when compounds of the invention were administered nasally (i.e. to the nasal mucosa).

Hemorrhoids are swellings caused by inflammation of the hemorrhoidal blood vessels found inside or around the rectum and the anus. Symptoms include bleeding (i.e. wounding) after the passage of a stool, prolapse of the hemorrhoid, mucus discharge and itchiness, soreness, redness and swelling in the area of the anus. Hemorrhoids are believed to be a consequence of an increase of pressure in the abdomen, for example, as a result of constipation or diarrhea.

Chronic obstructive pulmonary disease (COPD) is the name for a group of lung conditions that cause breathing difficulties, including emphysema (damage to the alveoli) and chronic bronchitis (long-term inflammation of the airways). COPD occurs when the lungs become inflamed, damaged and narrowed. The damage to the lungs is usually irreversible and results in an impairment of the flow of air into and out of the lungs. Symptoms of COPD include breathlessness, productive cough, frequent chest infections and persistent wheezing. The most common cause of the disease is smoking, although other risk factors include high levels of air pollution and occupational exposure to dust, chemicals and fumes.

Compounds of the invention may have positive effects in mitigating erythema, redness and swelling, edema, blisters, and bullous pemphigoid caused by various conditions including those mentioned generally and specifically herein, and may inhibit exudation of subcutaneous tissue fluid, and suppressing itching and pain caused by such inflammatory conditions.

Other inflammatory conditions that may be mentioned include:
(a) Mucosal inflammation, such as oral mucositis, aphthous ulcers, otitis media, laryngitis, tracheitis, esophagitis, gastritis, enteritis and enterocolitis (including bacillary dysentery, chronic amoebic dysentery, schistosomiasis, nonspecific ulcerative colitis and regional enteritis), cervicitis and endocervicitis, endometritis, inflammation caused by inhalation injury and the like, as well as mucosal inflammation associated with cancers, and infections (e.g. viral infections, such as the common cold or influenza), that affect mucosal surfaces, such as those in the oral cavity, the nasopharynx, the ear, the throat, the trachea, the gastrointestinal tract, the cervix, etc.
(b) Orthopedic inflammation associated with, for example bone fractures, pyogenic infection of bones and joints, inflammation caused by rheumatic bone diseases, as well as pyogenic osteomyelitis (acute, chronic, localized, sclerotic, post-traumatic), pyogenic arthritis; bone tumors (osteoma, osteoid osteoma, chondroma), bone cysts, osteoclastoma, primary bone sarcoma (osteosarcoma, chondrosarcoma, osteofibrosarcoma, Ewing's sarcoma, non-Hodgkin's lymphoma, myeloma, chordoma), metastatic bone tumors, tumor-like lesions of bone (bone cyst, aneurysmal bone cyst, eosinophilic granuloma, fibrous dysplasia); and rheumatic arthritis
(c) Nerve inflammation, such as peripheral polyneuritis, facial neuritis, peripheral neuritis, subcutaneous neuritis, ulnar neuritis, intercostal neuritis, etc.
(d) Subcutaneous and submucosal soft tissue inflammation, such as myositis, ligamentitis, tendonitis, panniculitis capsulitis, lymphadenitis, bubonadentitis, tonsillitis, synovitis, fasciitis, and soft tissue inflammation caused by injuries, contusion or laceration of muscles, ligaments, fascia, tendons, membrana synovialis, fat, articular capsules, and lymphoid tissue.
(e) Vascular inflammation, such as allergic leukocytoclastic vasculitis, allergic cutaneous vasculitis, polyarteritis nodosa, thrombotic vasculitis, granulomatous vasculitis, lymphocytic vasculitis, vasculitis with abnormalities in blood composition, and rheumatic vasculitis, as well as vascular inflammation associated with vascular cancers caused by allergic leukocytoclastic vasculitis, polyarteritis nodosa, thrombotic vasculitis, granulomatous vasculitis, lymphocytic vasculitis, vasculitis with abnormalities in blood composition, and rheumatic vasculitis.
(f) Inflammation of the internal organs, such as the heart, stomach, intestine, lung, liver, spleen, kidney, pancreas, bladder, ovary, and prostate, including but not limited to pericarditis, myocarditis, endocarditis, pneumonia, hepatitis, splenitis, nephritis pancreatitis, cystitis, oophoritis, prostatitis and treatment of gastric ulcer.
(g) Inflammation of the eye and surrounding area, such as conjunctivitis, keratitis (e.g. acute epithelial keratitis, nummular keratitis, interstitial keratitis, disciform keratitis, neurotrophic keratitis, mucous plaque keratitis, herpes simplex keratitis, herpes zoster keratitis, bacterial keratitis, fungal keratitis acanthamoebic keratitis, onchocercal keratitis, superficial punctate keratitis, ulcerative keratitis, exposure keratitis photokeratitis and contact lens acute red eye), optic neuritis, etc.
(h) Inflammation of the gums and the oral cavity, such as periodontitis, gingivitis, dental ulcers, etc.
(i) Inflammation associated with rheumatism, such as rheumatic vasculitis, rheumatoid arthritis, rheumatic bone diseases, ankylosing spondylitis, bursitis, Crohn's disease, gout, infectious arthritis, juvenile idiopathic arthritis, osteoarthritis, osteoporosis, polymyalgia rheumatica, polymyositis, psoriatic arthritis, scleroderma, Sjögren's syndrome, spondyloarthropathies, systemic lupus erythematosus, tendinitis, etc.

Compounds of the invention may also be used in the treatment of certain specific diseases of the digestive system, such as gastroesophageal reflux disease (GERD), which may be characterized by an acidic taste in the mouth, regurgitation, heartburn, pain with swallowing and/or sore throat, increased salivation (water brash), nausea, chest pain, and coughing. GERD may cause injury of the esophagus, including reflux esophagitis (i.e. inflammation of the esophageal epithelium which may cause ulceration at or around the junction of the stomach and esophagus), esophageal strictures (i.e. the persistent narrowing of the esophagus caused by reflux-induced inflammation), Barrett's esophagus (i.e. intestinal metaplasia (i.e. changes of epithelial cells from squamous to intestinal columnar epithelium of the distal esophagus) and/or esophageal adenocarcinoma (a form of cancer)).

Compounds of the invention may also be used in the treatment of certain specific diseases of the respiratory system, such as pulmonary cystic fibrosis, usual interstitial pneumonia, allergic pneumonia, asbestosis, emphysema, pulmonary heart disease, pulmonary embolism, etc. A specific disease state that may be mentioned in idiopathic pulmonary fibrosis (IPF).

IPF is a diffuse and fatal pulmonary interstitial disease with pathological features including alveolar epithelial damage, massive proliferation of lung fibroblasts, excessive deposition of extracellular matrix, ultimately leading to irreversible lung tissue damage. In the latter stages of the disease, subjects with IPF experience respiratory failure and death. It has been found that compounds of the invention may find utility in the treatment of IPF and/or alleviation of the symptoms associated with the disease.

Compounds of the invention are particularly useful in the treatment of the following lung and/or fibrotic conditions (whether otherwise mentioned herein or not): lung fibrosis, renal fibrosis, liver fibrosis, silicosis, acute bronchitis, chronic bronchitis, tracheobronchitis, bronchial asthma, status asthmatics, bronchiectasis, upper respiratory tract infections (including the common cold and influenza), allergic airway inflammation, bacterial pneumonia, viral pneumonia, mycoplasma pneumonia, reckettsia, radiation pneumonia, pneumococcal (including staphylococcal, streptococcal and gram-negative bacillus) pneumonia, pulmonary candidiasis (including aspergillosis, mucormycosis, histoplasmosis, actinomycosis and nocardiosis), pulmonary mycosis, cryptococcosis, lung abscesses, anaphylactic pneumonia, extrinsic allergic alveolitis, pulmonary eosinophilia (including Loeffler's syndrome and eosinophilosis), obstructive pulmonary emphysema, pulmonary edema, pulmonary tuberculosis, respiratory alkalosis/acidosis, acute lung injury, interstitial lung disease, empyema, lung fibroma and cor pulmonale.

Particular mucosal disorders and disease in which compounds of the invention find utility include anorectal diseases, such as diarrhea, hemorrhoids, abscesses, fistula, fissures, anal itching, anal sinusitis, warts and rectal prolapse; inflammatory bowel disease, including Crohn's disease and, particularly, ulcerative colitis; gynaecological diseases, such as cervicitis, vaginitis, pelvic pain and disorders; and dental diseases, such as paradentitis, for example.

Compounds of the invention may further possess an antioxidation effect, by increasing SOD (superoxide dismutase) production and reducing lipid oxidation. Compounds of the invention may therefore be considered to have antioxidant properties.

Compounds of the invention may also possess antipyretic properties that allow for the treatment of a fever and/or alleviate the symptoms thereof; for example, by reducing a subject's body temperature, which results in a reduction of fever. Compounds of the invention and formulations including them may therefore be considered to be antipyretics.

According to a further aspect of the invention there is provided a method of treatment of inflammation, of an inflammatory disorder, and/or of a disorder/condition characterised by inflammation (for example as a symptom), which method comprises the administration of a compound of the invention or a salt thereof to a patient in need of such treatment.

For the avoidance of doubt, in the context of the present invention, the terms ‘treatment’, ‘therapy’ and ‘therapy method’ include the therapeutic, or palliative, treatment of patients in need of, as well as the prophylactic treatment and/or diagnosis of patients which are susceptible to, inflammation and/or inflammatory disorders.

Compounds of the invention may further possess antiviral properties that may allow for the treatment of a viral infection per se, that is treatment of a viral infection, or a viral disease, by interfering with the replication of the virus within a host, as opposed to the treatment of any symptoms of any viral infection or disease, such as pain and/or inflammation. Such antiviral properties may also allow for the prevention of the onset of such an infection or disease, the protection of cells in a host from (e.g. further) viral infection, prevention or arrest of the spread of viral infection or disease (within a single host, or from one host to a new host), or for the prevention of reactivation of a virus after latency in a host.

According to a further aspect of the invention there is provided a method of treatment of a viral infection, which method comprises the administration of a compound of the invention or a salt thereof to a patient in need of such treatment.

Viral infections that may be mentioned include those caused by viruses in the following families: adenoviridae (e.g. adenovirus), papillomaviridae (e.g. human papillomavirus), polyomaviridae (e.g. BK virus; JC virus), herpesviridae (e.g. herpes simplex, type 1; herpes simplex, type 2; varicella-zoster virus; Epstein-Barr virus; human cytomegalovirus; human herpes virus, type 8), poxviridae (e.g. smallpox), hepadnaviridae (e.g. hepatitis B virus), parvoviridae (e.g. parvovirus B19), astroviridae (e.g. human astrovirus), caliciviridae (e.g. norovirus; Norwalk virus), picornaviridae (e.g. coxsackievirus, hepatitis A virus; poliovirus; rhinovirus), coronoviridae (e.g. severe acute respiratory syndrome virus), flaviviridae (e.g. hepatitis C virus; yellow fever virus; dengue virus; West Nile virus; tick-borne encephalitis virus), retroviridae (e.g. human immunodeficiency virus; HIV), togaviridae (e.g. rubella virus), arenaviridae (e.g. Lassa virus), bunyaviridae (e.g. hantavirus; Crimean-Congo hemorrhagic fever virus; Hantaan virus), filoviridae (e.g. Ebola virus; Marburg virus; Ravn virus), orthomyxoviridae (e.g. influenza viruses, including influenza A virus (e.g. H1N1 and H3N2 viruses), influenza B virus or influenza C virus), paramyxoviridae (e.g. measles virus; mumps virus; parainfluenza virus, respiratory syncytial virus), rhabdoviridae (e.g. rabies virus), hepeviridae (e.g. hepatitis E virus), reoviridae (e.g. rotavirus; orbivirus; coltivirus; Banna virus), as well as viruses not assigned to families, such as hepatitis D virus.

Viruses that may be more specifically mentioned include herpes simplex, type 1 and herpes simplex, type 2 viruses, human papillomavirus, influenza virus and parainfluenza virus.

Compounds of the invention may further possess antibacterial and/or bacteriostatic properties that may allow for the treatment of a bacterial infection per se, that is treatment of a bacterial infection, or a bacterial disease, by interfering with bacterial growth or proliferation in a host, as opposed to the treatment of any symptoms of any bacterial infection or disease, such as pain and/or inflammation. Compounds of the invention may therefore be considered to be bacteriocides and/or, preferably, bacteriostatic agents.

Such antibacterial properties may also allow for the prevention of the onset of such an infection or disease, the protection of cells in a host from (e.g. further) bacterial infection, prevention or arrest of the spread of bacterial infection or disease (within a single host, or from one host to a new host), or for the prevention of reactivation of a bacterium after latency in a host.

According to a further aspect of the invention there is provided a method of treatment of a bacterial infection, which method comprises the administration of a compound of the invention or a salt thereof to a patient in need of such treatment.

As disclosed herein, compounds of the invention may further possess anticancer properties that may allow for the treatment of a cancer per se, that is treatment of a cancer by interfering with the cancer as opposed to the treatment of any symptoms of the cancer, such as pain and/or inflammation. Such anticancer properties may also include the prevention of the onset of such a disease e.g. by treating inflammation and thereby preventing such onset.

According to another aspect of the invention, there is provided a method of treatment of cancer, which method comprises the administration of a compound of the invention or a salt thereof to a patient in need of such treatment.

Particular cancers that may be mentioned include oral cancer, a nasopharynx cancer, a middle ear cancer, a conjunctival cancer, a throat cancer, a tracheal cancer, an esophageal cancer, a gastric cancer, an intestinal cancer, a cervical cancer, an endometrial cancer, skin cancer and the like caused by oral mucositis, rhinitis, otitis media, conjunctivitis, pharyngitis, laryngitis, tracheitis, esophagitis, gastritis, enterocolitis, cervicitis, endometritis, erythema-like skin lesions and the like. A particular skin cancer that may be mentioned is basal cell carcinoma.

‘Patients’ include reptilian, avian and, preferably, mammalian (particularly human) patients.

In accordance with the invention, compounds of the invention are preferably administered locally or systemically, for example orally, intravenously or intraarterially (including by intravascular and other perivascular devices/dosage forms (e.g. stents)), intramuscularly, cutaneously, subcutaneously, transmucosally (e.g. sublingually or buccally), rectally, intravaginally, intradermally, transdermally, nasally, pulmonarily (e.g. tracheally or bronchially), preferably topically, or by any other parenteral route, in the form of a pharmaceutical preparation comprising the compound(s) in pharmaceutically acceptable dosage form(s).

Administration by inhalation (e.g. nasally) is particularly useful when the condition to be treated is rhinitis or inflammation resulting from viral infections of the airways (e.g. upper respiratory tract infections, such as the common cold and influenza).

Pulmonary administration is particularly useful when the condition to be treated is COPD or IPF. Topical forms of administration may be enhanced by creating a spray comprising active ingredients, e.g. by using a powder aerosol or by way of an aqueous mist using an appropriate atomisation technique or apparatus, such as a nebulizer.

Anorectal administration is particularly useful when the condition to be treated is hemorrhoids or ulcerative colitis, using an appropriate delivery means, such as a solution of foam to be injected or a suppository.

Administration to the lower gastrointestinal tract may also be achieved by parenteral, and particularly by peroral, delivery, by means of standard delayed- or extended-release coating techniques known to those skilled in the art. In particular, distinct parts of the upper or lower intestine may be targeted. For example, colonic administration can also be achieved by way of colon-targeted drug delivery means that are initially administered perorally or parenterally.

Compounds of the invention may in the alternative be administered by direct systemic parenteral administration. Such administration may be useful in methods of treatment of an inflammatory and/or fibrotic disorder or condition of one or more internal organs of a patient.

Internal organs that may be mentioned include the stomach, the intestines, the pancreas, the liver, the spleen, the bladder, the vascular system, the ovaries, the prostate, preferably the heart and the kidneys and more preferably the lungs.

Fibrotic conditions of internal organs that may be mentioned include acute and/or severe internal fibrotic conditions characterised by the excessive accumulation of fibrous connective tissues (as described above) in and around inflamed or damaged tissues. Formulations of the invention may thus be useful in the treatment or prevention of fibrogenesis (as described above) and the morbidity and mortality that may be associated therewith. Thus, (e.g. acute and/or severe) fibrotic conditions of the internal organs that may be treated with formulations of the invention include fibrosis of the liver, the kidneys, the lungs, the cardiovascular system, including the heart and the vascular system, the pancreas, the spleen, the central nervous system (nerve fibrosis), bone marrow fibrosis, the eyes, the vagina, the cervix, etc.

Inflammatory conditions of internal organs include any condition that is, or may develop into a condition that is, severe (i.e. one that requires intensive medical treatment), and in which some sort of inflammatory component is apparent, as may be characterised by detectable inflammation, and further in which morbidity is manifested (or is expected) and/or is life-threatening.

Inflammatory conditions that may be mentioned include one or more acute disorders or conditions of internal organs (i.e. one or more conditions that require, or may develop into a condition that requires, immediate medical interventions) that are characterized by inflammation (e.g. as a symptom), such as acute internal injuries, in one or more internal organs (including any of the organs mentioned hereinbefore). By treating such acute inflammatory disorders, formulations of the invention may prevent or arrest the development of symptoms (acute or chronic) that are associated with such conditions, and also may arrest the progress of morbidity and/or mortality that is associated with such conditions.

Acute inflammatory conditions that may be mentioned thus include conditions such as peritonitis, pancreatitis, colitis, proctitis, gastritis, duodenitis, pharyngitis, GERD, parodontitis and stomatitis. Particular acute inflammatory conditions that may be mentioned include acute injury to one or more internal organs (including any of those mentioned hereinbefore), such as acute lung injury, inhalation injury (such as burns), acute respiratory distress syndrome (ARDS), severe acute respiratory syndrome (SARS), and multiple-organ inflammation, injury and/or failure.

Such conditions may be caused by internal or external trauma (e.g. injury or a burn), or by an infection by e.g. viruses, bacteria or fungi.

For example, proctitis (which includes eosinophilic, gonorrheal and/or ulcerative proctitis) may be caused by inflammatory bowel disease, infections, radiation (e.g. for cancer), drugs such as antibiotics, surgery or allergic conditions, such as food intolerances.

For example, multiple-organ inflammation, injury and/or failure may result from extensive and/or traumatic external injuries, including traumatic and/or extensive external burns. Traumatic external burns will be understood to include second-degree, and more particularly third-degree burns and fourth-degree, burns. Extensive external burns will be understood to include burns that affect at least about 10%, such as at least about 15%, including at least about 20% of a patient's body area. External (and internal) burns may result from exposure to heat, chemicals and the like.

Acute inflammatory and/or fibrotic conditions may also result from sepsis or septic shock, which can be caused by viral, bacterial or fungal infection. Furthermore, acute lung injury, ARDS and, particularly, SARS may be caused by viruses, such as coronaviruses, include the novel SARS coronavirus 2 (SARS-CoV-2).

Thus, in addition, one or more of the aforementioned (e.g. acute) inflammatory conditions may (indeed in some cases will likely) result in some form of internal tissue damage and/or dysfunction of relevant internal tissues. Relevant tissues thus include (e.g. mucosal) tissues, such as the respiratory epithelium. Such tissue damage may also give rise to one or more of the fibrotic conditions mentioned hereinbefore. For example, the SARS disease caused by the novel coronavirus SARS-CoV-2 (coronavirus disease 2019 or COVID-19) is known in many cases to result in fibrosis, which arise from one or more of a number of factors, including inflammation.

In this respect, compounds of the invention and salts thereof find particular utility in the treatment of relevant inflammatory and/or fibrotic conditions on the basis that such conditions are often characterized by one or more comorbidities. By conditions that are ‘characterized by comorbidities’, we include that the main condition in question results in (or from) one more further medical conditions, including (and indeed preferably) those mentioned hereinbefore, at the same time, which conditions may interact and/or overlap with each other in some way.

Thus, there are provided:
methods of treatment of at least one inflammatory and/or fibrotic disorder or condition of one or more internal organs of a patient, which method comprises direct systemic parenteral administration of a compound of the invention, or a pharmaceutically-acceptable salt thereof, to a patient in need of such treatment;
a method of treatment of two or more inflammatory and/or fibrotic disorders or conditions of one or more internal organs of a patient, which method comprises direct systemic parenteral administration of a compound of the invention, or a pharmaceutically-acceptable salt thereof, to a patient in need of such treatment; and
a method of reduction in the incidence of morbidity and/or mortality that is or may be associated with one or more inflammatory and/or fibrotic disorders or conditions of one or more internal organs of a patient, which method comprises direct systemic parenteral administration of a compound of the invention, or a pharmaceutically-acceptable salt thereof, to a patient in need of such treatment.

When compounds of the invention/salts thereof are administered directly and parenterally, they may be administered intravenously, intraarterially, intravascularly, perivascularly, intramuscularly, cutaneously, and/or subcutaneously, for example by way of direct injection, or by way of any other parenteral route, in the form of a compound of the invention or salt thereof in the form of a pharmaceutically-acceptable dosage form.

Pharmaceutically-acceptable formulations for use in such administration may thus comprise compounds of the invention in admixture with a pharmaceutically-acceptable adjuvant, diluent or carrier, which may be selected with due regard to the intended route of direct parenteral administration and standard pharmaceutical practice. Such pharmaceutically-acceptable carriers may be chemically inert to the active compounds and may have no detrimental side effects or toxicity under the conditions of use. Such pharmaceutically-acceptable carriers may also impart an immediate, or a modified, release of the compound of the invention.

Formulations for injection may thus be in the form of an aqueous formulation such as an a suspension and/or, more preferably a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution), or a freeze-dried powder that may be reconstituted with a vehicle, such as an aqueous vehicle prior to use (e.g. injection)).

Formulations for injection may include other suitable excipients known to those skilled in the art, such as solvents (e.g. water), co-solvents, solubilizing agents (e.g. cyclodextrins), wetting agents, suspending agents, emulsifying agents, thickening agents, chelating agents, antioxidants, reducing agents, antimicrobial preservatives, bulking agents and/or protectants.

Formulations for injection are preferably buffered by standard techniques to physiologically-acceptable pH values (e.g. pHs of between about 4.5 and about 9.5, e.g. about 6 and about 9, such as between about 6.5 and about 8.5) using buffers and/or pH modifiers as described herein, and/or may further comprise tonicity-modifying agents (such as sodium chloride).

The above notwithstanding, preferred modes of delivery of compounds of the invention include topically to the site of inflammation (e.g. the mucosa, including the oral and/or nasal mucosa, the lung, the anorectal area and/or the colon or, more preferably, the skin) in an appropriate (for example pharmaceutically- and topically-acceptable) vehicle suitable for application to the skin and/or the appropriate mucosal surface, and/or a commercially-available formulation, but may also include oral, intravenous, cutaneous or subcutaneous, nasal, intramuscular, intraperitoneal, or pulmonary delivery.

Administration by injection is particularly useful for administering the compounds of the invention, in the form of a solution of suspension into e.g. the dermis (e.g. intradermal injection), joint cavity or the eyes.

Administration by intradermal injection (e.g. intradermally) is particularly useful for administering the compound of the invention, in the form of a solution or suspension (e.g. a dermal filler), into the dermis. This is particularly useful as a means of administration for melanin pigmentation therapy as described hereinbefore or for the use of the compounds of the invention in the treatment of, e.g. wrinkles.

Administration by injection is particularly useful to fill, e.g. the surgical site of the nasal cavity, the anal fistula, the space between the gingival and the root or the sinus. This is particularly useful for shaping support and/or lubrication.

Compounds of the invention will generally be administered in the form of one or more for example pharmaceutical formulations in admixture with a (e.g. pharmaceutically acceptable) adjuvant, diluent or carrier, which may be selected with due regard to the intended route of administration (e.g. topical to the relevant mucosa (including the lung) or, preferably, the skin) and standard pharmaceutical or other (e.g. cosmetic) practice. Such pharmaceutically acceptable carriers may be chemically inert to the active compounds and may have no detrimental side effects or toxicity under the conditions of use. Such pharmaceutically acceptable carriers may also impart an immediate, or a modified, release of the compound of the invention.

Suitable pharmaceutical formulations may be commercially available or otherwise prepared according to techniques that are described in the literature, for example, Remington The Science and Practice of Pharmacy, 22^{nd} edition, Pharmaceutical Press (2012) and Martindale—The Complete Drug Reference, 38^{th} Edition, Pharmaceutical Press (2014) and the documents referred to therein, the relevant disclosures in all of which documents are hereby incorporated by reference. Otherwise, the preparation of suitable formulations including compounds of the invention may be achieved non-inventively by the skilled person using routine techniques.

Compounds of the invention may be in the form of an aqueous formulation such as an emulsion, a suspension and/or a solution (e.g. an (optionally) buffered aqueous formulation (e.g. solution), such as a physiological saline-containing formulation (e.g. solution), a phosphate-containing formulation (e.g. solution), an acetate-containing formulation (e.g. solution) or a borate-containing formulation (e.g. solution)), or a freeze-dried powder.

Compounds of the invention may further and/or in the alternative be combined with appropriate excipients to prepare:
gel formulations (for which suitable gel matrix materials include cellulose derivatives, carbomer and alginates, gummi tragacanthae, gelatin, pectin, carrageenan, gellan gum, starch, Xanthan gum, cationic guar gum, agar, noncellulosic polysaccharides, saccharides such as glucose, glycerin, propanediol, vinyl polymers, acrylic resins, polyvinyl alcohol, carboxyvinyl polymer and, particularly, hyaluronic acid);
lotions (for which suitable matrix materials include cellulose derivatives, glycerin, noncellulosic polysaccharides, polyethylene glycols of different molecular weights and propanediol);
pastes or ointments (for which suitable paste matrix materials include glycerin, vaseline, paraffin, polyethylene glycols of different molecular weights, etc.);
creams or foams (for which suitable excipients (e.g. foaming agents) include hydroxypropyl methyl cellulose, gelatin, polyethylene glycols of different molecular weights, sodium dodecyl sulfate, sodium fatty alcohol polyoxyethylene ether sulfonate, corn gluten powder and acrylamide);
powder aerosols (for which suitable excipients include mannitol, glycine, dextrin, dextrose, sucrose, lactose, sorbitol and polysorbates, e.g. a dry powder inhalant); and/or
liquid, for example, water (aerosol) sprays for oral use or for inhalation (for which suitable excipients include viscosity modifiers, such as hyaluronic acid, sugars, such as glucose and lactose, emulsifiers, buffering agents, alcohols, water, preservatives, sweeteners, flavours, etc.);
injectable solutions or suspensions (which may be aqueous or otherwise and for which suitable excipients include solvents and co-solvents, solubilizing agents, wetting agents, suspending agents, emulsifying agents, thickening agents, chelating agents, antioxidants, reducing agents, antimicrobial preservatives, buffers and/or pH modifiers, bulking agents, protectants and tonicity-modifying agents), particular injectable solutions or suspensions that may be mentioned include dermal fillers (i.e. injectable fillers or soft-tissue fillers), particularly when the compound of the invention is combined with hyaluronic acid.

Moisturizing agents, such as glycerol, glycerin, polyethylene glycol, trehalose, glycerol, petrolatum, paraffin oil, silicone oil, hyaluronic acid and salts (e.g. sodium and potassium salts) thereof, octanoic/caprylic triglyceride, and the like; and/or antioxidants, such as vitamins and glutathione; and/or pH modifiers, such as acids, bases and pH buffers, may also be included in such formulations, as appropriate. Furthermore, surfactants/emulsifiers, such as hexadecanol (cetyl alcohol), fatty acids (e.g. stearic acid), sodium dodecyl sulfate (sodium lauryl sulfate), sorbitan esters (e.g. sorbitan stearate, sorbitan oleate, etc.), monoacyl glycerides (such as glyceryl monostearate), polyethoxylated alcohols, polyvinyl alcohols, polyol esters, polyoxyethylene alkyl ethers (e.g. polyoxyethylene sorbitan monooleate), polyoxyethylene castor oil derivatives, ethoxylated fatty acid esters, polyoxylglycerides, lauryl dimethyl amine oxide, bile salts (e.g. sodium deoxycholate, sodium cholate), lipids (e.g. fatty acids, glycerolipids, glycerophospholipids, sphingolipids, sterols, prenols, saccharolipids, polyketides), phospholipids, N,N-dimethyldodecylamine-N-oxide, hexadecyltrimethyl-ammonium bromide, poloxamers, lecithin, sterols (e.g. cholesterol), sugar esters, polysorbates, and the like; preservatives, such as phenoxyethanol, ethylhexyl glycerin, and the like; and thickeners, such as acryloyldimethyltaurate/VP copolymer, may be included. In particular, stearic acid, glyceryl monostearate, hexadecanol, sorbitan stearate, cetyl alcohol, octanoic/capric glyceride etc. may be included, particularly in cream formulations.

Compounds of the invention, and (e.g. pharmaceutical) formulations (e.g. aqueous solutions, gels, creams, ointments, lotions, foams, pastes and/or dry powders as described above) including them, may further be combined with an appropriate matrix material to prepare a dressing or a therapeutic patch for application on a biological surface, such as the skin or a mucosal surface. Such formulations may thus be employed to impregnate a matrix material, such as gauze, non-woven cloth or silk paper. The therapeutic patch may alternatively be, for example, a band-aid, a facial mask, an eye mask, a hand mask, a foot mask, etc.

Vaseline may be employed for use in applying such dressings to wounds, but we have also found that ointments based on PEGs (e.g. PEG 400) may be combined with matrix materials to prepare dressings without the need to use Vaseline.

Compounds of the invention may also be used in combination with solid supports (such as nasal dressings (for example, to stop nasal bleeding), dermal scaffolds (for example, in wound healing) or artificial bones (for example, in the case of bone grafting/implantation).

Compounds of the invention may be administered for inhalation by way of suspension, a dry powder or a solution. Suitable inhalation devices include pressurized metered-dose inhalers (pMDIs), which may be hand- or breath-actuated and employed with or without a standard spacer device, dry powder inhalers (DPIs), which may be single-dose, multi-dose, and power-assisted, and soft mist inhalers (SMIs) or nebulizers, in which aerosol drug in a fine mist is delivered with slower velocity than a spray delivered using, for example, a pMDI.

In pMDIs, compounds of the invention may be administered as a pressurized suspension of micronized particles distributed in a propellant (e.g. HFA, along with excipients, such as mannitol, lactose, sorbitol, etc.), or as an ethanolic solutions, to deliver one or more metered dose of between about 20 and about 100 μL with each actuation. Actuation may be effected by hand (e.g. pressing) or by inhalation (breath-actuation), involving a flow-triggered system driven by a spring.

In DPIs, compounds of the invention may be administered in the form of micronized drug particles (of a size between about 1 and about 5 μm), either alone or blended with inactive excipient of larger particle size (e.g. mannitol), inside a capsule, which may be pre-loaded or manually loaded into the device. Inhalation from a DPI may de-aggregate the medication particles and disperse them within the airways.

In SMIs, compounds of the invention may be stored as a solution inside a cartridge, which is loaded into the device. A spring may release the dose into a micropump, such that the dose is released when a button is pressed, releasing jet streams of drug solution.

Various nebulizers may also be used to administer compounds of the invention in the form of a fine mist of aerosolized solution. Nebulizers may include breath-enhanced jet nebulizer (in which, with the assistance of a compressor, an air stream moves through jet causing drug solution to be aerosolized); breath-actuated jet nebulizers (in which, after a patient inhales, with the assistance of a compressor, an air stream moves through a tube causing the drug solution to be aerosolized); ultrasonic nebulizers (in which piezoelectric crystals vibrate causing aerosolization by heating causing nebulization); vibrating mesh nebulizers (in which piezoelectric crystals vibrate a mesh plate causing aerosolization to give very fine droplets without a significant change in temperature of the solution during nebulization).

According to a further aspect of the invention there is provided a process for the preparation of a pharmaceutical composition/formulation, as defined herein, which process comprises bringing into association a compound of the invention, as hereinbefore defined, with one or more pharmaceutically-acceptable excipient, as hereinbefore defined.

Compounds of the invention may also be combined in treatment with one or more growth factors selected from platelet-type growth factors (including platelet-derived growth factors, PDGFs); osteosarcoma-derived growth factors (ODGF), epidermal growth factors (EGFs), transforming growth factors (TGFα and TGFβ), fibroblast growth factors (αFGF, βFGF), insulin-like growth factors (IGF-I, IGF-II), nerve growth factors (NGF), interleukin-type growth factors (IL-1, IL-1, IL-3), erythropoietin (EPO), and colony stimulating factor (CSF).

According to a further aspect of the invention there is provided a (e.g. pharmaceutical) composition comprising a compound of the invention and one or more pharmaceutically-acceptable excipient, such as an adjuvant, diluent or carrier. Preferred formulations are suitable for application locally to e.g. the mucosa (including the oral and/or nasal mucosa, the lung, the anorectal area and/or the colon) or, more preferably, the skin and therefore comprise a topically-acceptable adjuvant, diluent or carrier.

There is, thus, further provided pharmaceutical compositions comprising compounds of the invention that are suitable for, adapted for, and/or packaged and presented for topical administration (e.g. to the mucosa, including the oral and/or nasal mucosa, the lung, the anorectal area and/or the colon, or, preferably, to the skin), as well as the use of such a formulation in the treatment of a disorder including inflammation, an inflammatory disorder and/or a condition characterized by inflammation (e.g. as a symptom) by way of direct topical administration of that formulation (e.g. to the mucosa, including the oral and/or nasal mucosa, the lung, the anorectal area and/or the colon, or, preferably, to the skin).

In relation to this aspect of the invention, for the avoidance of doubt, topical formulations comprising compounds of the invention may be used in any and all conditions described herein, including treatments of inflammation, in the treatment of any and all inflammatory disorder(s), and/or in the treatment of any and all condition(s) characterized by inflammation, as hereinbefore mentioned, defined or described. Similarly, topical formulations comprising compounds of the invention that may be mentioned include any and all of those mentioned, defined or described herein. Any and all of the relevant disclosures herein are hereby incorporated by reference in conjunction with this aspect of the invention.

Topical (e.g. liquid- or (e.g. aqueous) solution-based) formulations comprising compounds of the invention may be particularly useful in wound recovery, and may alleviate pain (including aching) and, particularly, pruritis/itching that is associated with the wound itself and the wound healing process. Such topical formulations comprising compounds of the invention may be particularly useful in the prevention and/or suppression of the exudation of body fluids from wounds, particularly during the acute inflammation stage, for example during the first 48 hours, after a burn or wound has been inflicted. This prevents the risk of infection, and other physiological reactions. Such topical formulations comprising compounds of the invention may also be particularly useful in the prevention and/or suppression of scarring and melanin pigmentation (vide supra), whether associated with wounds or otherwise.

Administration of compounds of the invention may be continuous or intermittent. The mode of administration may also be determined by the timing and frequency of administration, but is also dependent, in the case of the therapeutic treatment of inflammation, on the severity of the condition.

Depending on the disorder, and the patient, to be treated, as well as the route of administration, compounds of the invention may be administered at varying therapeutically effective doses to a patient in need thereof.

Similarly, the amount of compound of the invention in a formulation will depend on the severity of the condition, and on the patient, to be treated, but may be determined by the skilled person.

In any event, the medical practitioner, or other skilled person, will be able to determine routinely the actual dosage, which will be most suitable for an individual patient, depending on the severity of the condition and route of administration. The dosages mentioned herein are exemplary of the average case; there can, of course, be individual instances where higher or lower dosage ranges are merited, and such are within the scope of this invention.

Doses may be administered between once and four (e.g. three) times daily.

Appropriate concentrations of compounds of the invention in an aqueous solution product may be about 0.01 (e.g. about 0.1) to about 15.0 mg/mL, in all cases calculated as the free (non-salt) compound.

Appropriate topical doses of compounds of the invention are in the range of about 0.05 to about 50 μg/cm² of treated area, such as about 0.1 (e.g. about 0.5) to about 20 μg/cm² of treated area, including about 1 to about 10 μg/cm² of treated area, such as about 5 μg/cm² of treated area, in all cases calculated as the free (non-salt) compound.

Appropriate doses of compounds of the invention for nasal administration (e.g. by inhalation) are in the range of about 0.01 μg to about 2000 mg, for example between about 0.1 μg to about 500 mg, or between 1 μg to about 100 mg. Particular doses for nasal administration that may be mentioned include between about 10 μg to about 1 mg, particularly a dose of about 0.1 mg (i.e. about 100 μg). Nasal administration of about 0.1 mg per day of compounds of the invention has been found to be particularly effective in the treatment of conditions associated with inflammation of the nasal passages and mucosae, such as rhinitis (e.g. allergic rhinitis) and/or conditions associated with nasosinusitis surgery.

Appropriate doses of compounds of the invention for pulmonary administration (e.g. by inhalation) are in the range of about 0.01 μg to about 2000 mg, for example between about 0.1 μg to about 500 mg, or between 1 μg to about 100 mg. Particular doses for pulmonary administration that may be mentioned include between about 10 μg to about 10 mg, particularly a dose of about 0.6 mg (i.e. 60 μg) to 6 mg (e.g. for use in treating COPD or IPF).

We prefer that pH values of formulations comprising compounds of the invention are in the range of about 1.0 to about 9.0 (for example about 3.0 to about 8.0).

In any event, the dose administered to a mammal, particularly a human, in the context of the present invention should be sufficient to effect a therapeutic response in the mammal over a reasonable timeframe (as described hereinbefore). One skilled in the art will recognize that the selection of the exact dose and composition and the most appropriate delivery regimen will also be influenced by inter alia the pharmacological properties of the formulation, the nature and severity of the condition being treated, and the physical condition and mental acuity of the recipient, as well as the age, condition, body weight, sex and response of the patient to be treated, and the stage/severity of the disease, as well as genetic differences between patients.

Compounds of the invention are useful in human and animal medicine. In this respect, and as described above, compounds of the invention that possess an appropriate degree of relevant pharmacological (or biological) activity per se may be used as human, and/or animal, medicines.

Certain compounds of invention, particularly compounds of formula I, and/or linear long-chain compounds of the invention preferably in which long-chain compounds W represents HCA, HCA-Ala or, more preferably, DOPA or DOPA-Ala, and/or U represents DOPA may in addition and/or instead of possessing the aforementioned biological activity, possess adhesive properties.

These adhesive properties stem from the fact that the relevant W and/or U groups are capable of cross-linking with each other in order to form three-dimension networks.

Such compounds of the invention may adhere to a number of substrates including inorganic substrates, such as glass, metal and the like, as well as organic substrates, such as biological tissue.

In respect, such compounds of the invention may also be used as wound surface repair products, wound surface protecting products, medical biological adhesive products, medical coating products, industrial coating products (e.g. in corrosion prevention in ships, electronic apparatuses, pipelines and the like), biochemical reagents, medical products, sterilization products, culture vessels for cell culture and the like.

Such compounds of the invention may form a film over various skin and mucous wound surfaces such as burns, scalds, ulcers, chilblains, and bedsores to aid in recovery. Such compounds of the invention may also be used in surgery, e.g. in the closure of surgical incisions, adhesion of fractured bones, adhesion of mucous membranes, coatings of human body implants such as artificial bones, cartilage brackets, periostea, artificial joints, dental implants, plugging stents, spinal fusion devices, spinal spacers and organ patches.

According to a further aspect of the invention, there is provided a compound of formula I and/or a linear long-chain compounds of the invention, preferably in which linear long-chain compounds W represents HCA, HCA-Ala or, more preferably, DOPA or DOPA-Ala, and U represents DOPA as an adhesive or a film-forming material.

As discussed hereinbefore, naturally occurring MAP is known for its adhesive properties, but it should be remembered that such adhesives properties may arise from the fact that that is a high molecular weight, linear peptide that can exist in multiple conformations, enabling inter- and intramolecular reactions/cross-linking of DOPA residues in molecules, and thereby adhesion. Conversely, compounds of the invention as defined above are not linear polypeptides or proteins but are instead, for example multiply-branched lower molecular weight residues and it is a surprise to the applicant that similar properties (whether adhesive or biological) to naturally-occurring MAP are observed.

Such crosslinking may be carried out by a variety of chemicals (e.g. iodine vapour, glutaraldehyde, N-(3-dimethylaminopropyl)-N′-ethylcarbodiimide hydrochloride and N-hydroxysuccinimide (EDC/NHS), 4-(4,6-dimethoxy-1,3,5-triazin-2-yl)-4-methylmorpholinium chloride (DMTMM), or other water soluble condensation agents) or enzymatic means (e.g. tyrosinase, or as described hereinafter).

Irrespective of the level of pharmacological activity that compounds of the invention may possess, they may in any event be (and/or may be further) combined with active pharmaceutical ingredients, either in combination therapy (as described hereinafter), or by performing a function either as, or as part of, a pharmaceutically-acceptable excipient (e.g. an adjuvant, diluent or carrier), as part of a medical device, and/or as part of a drug-medical device combination.

Certain compounds of the invention may thus be described as novel multifunctional excipients, which may be used for a variety of applications in the pharmaceutical field. In this respect, such compounds of the invention include those that may be used as adhesives and/or as film-forming agents (as described hereinbefore), Furthermore, such compounds of the invention and/or different compounds of the invention may in the alternative, and/or in addition, be used as release retarding polymers, as binders, as suspending agents, as gelling agents, as coating agents, as diluents or as carriers for active ingredients (drugs) of varying solubilities.

Compounds of the invention that are particularly useful as pharmaceutical excipients may be adopted for large-scale production and may present no significant toxicity risk and may so be described and listed as ‘Generally Recognized as Safe’ (GRAS) by the US Food & Drug Administration (FDA).

Such compounds of the invention may also be employed as excipients in veterinary science, as well as in cosmetics.

According to a further aspect of the invention, there is provided a pharmaceutical formulation comprising an active pharmaceutical ingredient in admixture with a pharmaceutically-acceptable excipient system (such as a pharmaceutically-acceptable adjuvant, diluent or carrier system), which excipient system comprises one of more compounds of the invention.

Furthermore, compounds of the invention may be combined with active pharmaceutical ingredients, and may thus be employed as part of a drug-medical device combination, which combination comprises one or more active pharmaceutical ingredients and one or more compounds of the invention, in which said one or more compounds of the invention constitute the medical device component of that combination.

When used as, or as part of, a medical device or the medical device part of a drug-medical device combination, the skilled person will understand that the relevant compound of the invention will be employed in human or animal medicine, optionally in conjunction with an active pharmaceutical ingredient, in such a way as to affect the structure, and/or one or more functions, of a human or an animal body, and will achieve its primary intended purposes without exerting a chemical action within or on said human or animal body (optionally in a manner that is not dependent upon the compound of the invention being metabolized for the achievement of any of its primary intended purposes).

In this respect, compounds of the invention may be combined with a multitude of known pharmaceutically-active ingredients and may be so combined irrespective of whether the compound of the invention is employed:
as a separate pharmaceutically-active ingredient per se in combination therapy;
as, or as part of, a medical device;
as, or as the medical device part of, a drug-medical device combination; or
as a pharmaceutically-acceptable excipient.

Such patients may also (and/or may already) be receiving therapy based upon administration of one or more of such other, known pharmaceutically-active ingredients, by which we mean receiving a prescribed dose of one or more of the active ingredients mentioned herein, prior to, in addition to, and/or following, treatment with a compound of the invention.

Pharmaceutically-active agents that may be co-administered with a compound of the invention include any agent, or drug, that is capable of producing some sort of physiological effect (whether in a therapeutic or prophylactic capacity against a particular disease state or condition) in a living subject, including, in particular, mammalian and especially human subjects (patients).

In addition, compounds of the invention such as those that may be crosslinked as hereinbefore described may be employed as pharmaceutical excipients and may be mixed with such pharmaceutically-active ingredients either before or after crosslinking and/or at least partial crosslinking, as hereinbefore described, in order to form a stable pharmaceutical composition in which a compound of the invention acts an excipient, such as a carrier. When employed in this way, it may be found that compounds of the invention may affect, in a positive way, physical, chemical and/or biological properties of such active ingredients, including their physical and/or chemical stability and/or their metabolism following administration.

Pharmaceutically-active agents that may be used along with compounds of the invention may, for example, be selected from anti-inflammatory agents, pro-inflammatory agents, antibiotics, anti-bacterial and/or antiprotozoal agents, antiviral agents (e.g. protease inhibitors), anaesthetics and wound recovery drugs (e.g. growth factors).

Biologically-active agents may, for example, be selected from anti-inflammatory agents, pro-inflammatory agents, antibiotics, anti-bacterial and/or antiprotozoal agents, antiviral agents (e.g. protease inhibitors), anaesthetics and wound recovery drugs (e.g. growth factors).

Non-limiting examples of anti-inflammatory drugs which may be used also include those used in the treatment of rheumatic diseases and/or arthritis (such as cataflam, betamethasone, naproxen, cyclosporin, chondroitin, celecoxib, etodolac, meclofenamate, salsalate, methylprednisolone, and piroxicam); osteoarthritis (such as sulindac, meloxicam, fenoprofen, etoricoxib, and nabumetone); inflammation and its symptoms, e.g. fever, pain, itchiness and/or swelling (such as mefenamic acid, indomethacin, aspirin, ketorolac, fluorometholone, loteprednol, hydrocortisone, fluorometholone, bromfenac, prednisolone acetate, indomethacin, and ibuprofen); allergies and their symptoms (such as pheniramine, diphenhydramine, naphazoline, antazoline, prednisolone, lodoxamide, pemirolast, oxymetazoline, ketotifen, naphazoline, emestine fumarate, olopatadine, azelastine, tranilast, levocabastine, cortisone, ephedrine, cetirizine, levocetirizine, pseudophedrine, fexofenadine, terfenadine, loratadine, and alexis); respiratory diseases, including asthma and/or COPD (such as budesonide, ciclesonide, nedocromil, dexamethasone, ambroxol, and pranlukast); skin diseases (such as mometasone, triamcinolone, desonide, sulfacetamide, tacrolimus, allantoin, and triamcinolone); mastocytosis (such as cromolyn); gout (such as diclofenac, and febuxostat); conjunctivitis (such as hydrobenzole, pranoprofen, and zinc sulfate); eye diseases (such as dextran 70, thyroxine/liothyronine, and ocular extractives), known or commercially-available pharmaceutically acceptable salts of any of the foregoing, and combinations of any of the forgoing compounds and/or salts.

Antiinflammatory drugs that may be mentioned include endogenous (and/or exogenous) lipid-based pro-resolving, antiinflammatory molecules or mediators, such as lipoxins, resolvins, and protectins. Pro-inflammatory agents that may be mentioned include prostaglandins (e.g. latanoprost, prostaglandin E1, and prostaglandin E2), and leukotrienes (e.g. Leukotriene B4).

Non-limiting examples of anti-bacterial drugs which may be used also include chloramphenicol, ofloxacin, levofloxacin, tobramycin, norfloxacin, ciprofloxacin, lomefloxacin, lincomycin, fluconazole, enoxacin, furazolidone, nitrofurazone, rifampicin, micronomicin, gentamicin, cetylpyridinium, neomycin, roxithromycin, sulfadiazine silver, clarithromycin, clindamycin, metronidazole, azithromycin, mafenide, sulfamethoxazole, paracetamol, chloramphenicol, pseudoephedrine, mupirocin, amoxicillin, amoxicillin/clavulanic acid, trimethoprim/sulfamethoxazole, cefalexin, moxifloxacin, known or commercially-available pharmaceutically acceptable salts of any of the foregoing, and combinations of any of the foregoing compounds and/or salts.

Non-limiting examples of antiviral drugs which may be used also include tobramycin ribavirin, acyclovir, moroxydine, foscarnet, ganciclovir, idoxuridine, trifluridine, brivudine, vidarabine, entecavir, telbivudine, foscarnet, zidovudine, didanosine, zalcitabine, stavudine, lamivudine, abacavir, emtricitabine, nevirapine, delavirdine, efavirenz, etravirine, rilpivirine, saquinavir, ritonavir, indinavir, nelfinavir, amprenavir, lopinavir, ritonavir, atazanavir, fosamprenavir, tipranavir, darunavir, telaprevir, boceprevir, simeprevir, asunaprevir, raltegravir, elvitegravir, dolutegravir, rsv-igiv, palivizumab, docosanol, enfuvirtide, maraviroc, vzig, varizig, acyclovir, ganciclovir, famciclovir, valacyclovir, penciclovir, valganciclovir, cidofovir, tenofovir disoproxil fumarate, adefovir dipivoxil, fomivirsen, podofilox, imiquimod, sinecatechins, interferon-α 2b (recombinant, human), known or commercially-available pharmaceutically acceptable salts of any of the foregoing, and combinations of any of the foregoing compounds and/or salts.

Non-limiting examples of anaesthetics which may be used also include articaine, dextropropoxyphene, sevoflurane, cophenylcaine, lidocaine, prilocaine, pramoxine, benzocaine, dibucaine, diclonine, tetracaine, bupivacaine and known or commercially-available pharmaceutically acceptable salts of any of the foregoing, and combinations of any of the foregoing compounds and/or salts.

Non-limiting examples of wound recovery drugs which may be used also include basic fibroblast growth factor (recombinant, human; recombinant, bovine), epidermal growth factor (recombinant, human; yeast), rhEFG (I), acidic fibroblast growth factor (recombinant, human), granulocyte macrophage stimulating factor (recombinant, human), sulfadiazine silver, sulfadiazine zinc, fusidic acid, bacitracin, chlorhexidine, silver nitrate, triethanolamine, ethacridine, retinoids, calf blood deproteinized extract, carraghenates, amiotide and known or commercially-available pharmaceutically acceptable salts of any of the foregoing, and combinations of any of the foregoing compounds and/or salts.

Such pharmaceutically-active ingredients include those that may be administered topically, e.g. to the skin or to a mucosal surface along with a compound of the invention. In this respect, preferred active ingredients from the above list include cyclosporin, chondroitin, loteprednol, fluorometholone, bromfenac, prednisolone acetate, indomethacin, oxymetazoline, ketotifen, naphazoline, emestine fumarate, olopatadine, azelastine, tranilast, levocabastine, cortisone, ephedrine, cetirizine, pseudoephedrine, levocetirizine, fexofenadine, terfenadine, loratadine, alexis, dexamethasone, ambroxol), sulfacetamide, tacrolimus, allantoin, triamcinolone, cromolyn, nedocromil, diclofenac, hydrobenzole, pranoprofen, zinc sulfate, dextran 70, thyroxine/liothyronine, ocular extractives, chloramphenicol, ofloxacin, levofloxacin, tobramycin, norfloxacin, ciprofloxacin, lomefloxacin, lincomycin, fluconazole, enoxacin, furazolidone, nitrofurazone, rifampicin, micronomicin, gentamicin, cetylpyridinium, neomycin, roxithromycin, sulfadiazine silver, clarithromycin, sulfamethoxazole, chloramphenicol, tobramycin ribavirin, acyclovir, moroxydine, foscarnet, ganciclovir, interferon-α 2b (recombinant, human), articaine, dextropropoxyphene, sevoflurane, cophenylcaine, lidocaine, prilocaine, pramoxine, benzocaine, dibucaine, diclonine, tetracaine, bupivacaine, basic fibroblast growth factor (recombinant, human; recombinant, bovine), epidermal growth factor (recombinant, human; yeast), rhEFG (I), acidic fibroblast growth factor (recombinant, human), granulocyte macrophage stimulating factor (recombinant, human), sulfadiazine silver, sulfadiazine zinc, fusidic acid, bacitracin, chlorhexidine, silver nitrate, triethanolamine, ethacridine, retinoids, calf blood deproteinized extract, carraghenates, amiotide, and known or commercially-available pharmaceutically acceptable salts of any of the foregoing, and combinations of any of the foregoing compounds and/or salts.

Other pharmaceutically-active ingredients that may be co-administered with a compound of the invention include those that may be administered to treat one or of the gastrointestinal disorders mentioned hereinbefore.

Non-limiting examples of gastrointestinal drugs include oxalazine (olsalazine), sulfasalazine, domperidone, erythromycin, berberine, dexamethasone, cefuroxime axetil, levofloxacin, mesalazine, belladonna, sulfobenzidine, azathioprine, sulfasalazine, live bacillus (such as Clostridium butyricum, licheniformis, cereus), probiotics (such as bifidobacterium) tegafur, nifuratel, amoxicillin, ampicillin, nystatin, allicin, cefadroxil, dyclonine, carmofur, fluorouracil, mosapride, sodium carbosulfan, thrombin, pantoprazole, cimetidine, cisapride, ethylenediamine diacetamine, nimustine, famotidine, barium sulfate, aminocaproic acid, roxatidine acetate, vincristine, azasetron, lentinan, bismuth salts (e.g. aluminate, potassium citrate) in combination with e.g. magnesium salts, magnesium trisilicate, bicarbonate, vitamin U, aluminium hydroxide, belladonna extract, famotidine and calcium carbonate, magnesium hydroxide, hydrotalcite, proton pump inhibitors (such as omeprazole, lansoprazole, rabeprazole, pantoprazole, dexlansoprazole or esomeprazole), glycine, trypsin, allantoin aluminium hydroxide, sodium L-glutamine gualenate, rebampette, rotundine, quxipite, lafutidine, thymus protein, hericium erinaceus, irsogladine maleate, nizatidine, L-glutamine and sodium azulene sulfonate (sodium gualenate), ranitidine, bismuth citrate, lactobacillin, bisacordine, dimethylsiloxane, live Clostridium butyricum, loperamide hydrochloride, dibazol, secnidazole, zinc acephate, montmorillonite, tegafur/gimeracil/oteracil, famotidine, oteracil, doxifluridine, capecitabine and known or commercially-available pharmaceutically acceptable salts of any of the foregoing.

Pharmaceutically-active ingredients that may be mentioned for use in combination with compounds of the invention include active ingredients that are useful in the treatment of inflammation and/or inflammatory disorders (other anti-inflammatory agents).

Anti-inflammatory agents that may be used in combination with compounds of the invention in the treatment of inflammation include therapeutic agents that are useful in the treatment of inflammation and/or of diseases characterized by inflammation as one of its symptoms, including those described hereinbefore. Depending on the condition to be treated, such anti-inflammatory agents may include NSAIDs (e.g. aspirin), aminosalysates (e.g. 5-aminosalicyclic acid (mesalazine)), leukotriene receptor antagonists (e.g. montelukast, pranlukast, and zafirlukast), corticosteroids, analgesics and certain enzymes, such as trypsin, for example as described hereinafter. Compounds of the invention may also be combined with leukotrienes (e.g. cysteinyl leukotrienes, and leukotriene B4).

Other preferred agents that may be combined with compounds of the invention include LTB4 (to treat wounds and burns), NSAIDS (e.g. aspirin) or montelukast (to treat inflammation generally) and trypsin (to treat inflammation of the mucosa associated with e.g. viral infections).

Compounds of the invention may also be combined with other therapeutic agents which, when administered, are known to give rise to inflammation as a side-effect.

Conjugates of the invention may also be combined with stem cells (e.g. totipotent (omnipotent), pluripotent (such as embryonic or induced pluripotent stem cells), multipotent (such as mesenchymal stem cells), oligopotent (such as hematopoietic stem cells), or unipotent (such as muscle stem cells)).

Other known pharmaceutically-active ingredients may also be administered in combination with compounds of the invention in numerous ways.

For example, compounds of the invention may be ‘combined’ with the (or with the other) pharmaceutically-active ingredients (or ‘therapeutic agents’) for administration together in the same (e.g. pharmaceutical) formulation, or administration separately (simultaneously or sequentially) in different (e.g. pharmaceutical) formulations.

Thus, such combination products provide for the administration of compounds of the invention in conjunction with the (or with the other) therapeutic agent, and may thus be presented either as separate formulations, wherein at least one of those formulations comprises a compound of the invention, and at least one comprises the (or the other) therapeutic agent, or may be presented (i.e. formulated) as a combined preparation (i.e. presented as a single formulation including a compound of the invention and the (or the other) therapeutic agent).

Thus, there is further provided:
(1) a (e.g. pharmaceutical) formulation including a compound of the invention; another pharmaceutically-active ingredient; and, optionally, a pharmaceutically-acceptable inactive excipient (e.g. adjuvant, diluent or carrier), which formulation is hereinafter referred to as a ‘combined preparation’; and
(2) a kit of parts comprising components:
(A) a compound of the invention, optionally in the form of an (e.g. pharmaceutical) formulation in admixture with a pharmaceutically-acceptable inactive excipient (e.g. adjuvant, diluent or carrier); and
(B) another pharmaceutically-active ingredient, optionally in the form of a (e.g. pharmaceutical) formulation in admixture with a pharmaceutically-acceptable adjuvant, diluent or carrier,
which components (A) and (B) are each provided in a form that is suitable for administration in conjunction with the other.

In a further aspect of the invention, there is provided a process for the preparation of a combined preparation (1) as hereinbefore defined, which process comprises bringing into association a compound of the invention, the other pharmaceutically-active ingredient, and at least one (e.g. pharmaceutically-acceptable) excipient.

In a further aspect of the invention, there is provided a process for the preparation of a kit-of-parts (2) as hereinbefore defined, which process comprises bringing into association components (A) and (B). As used herein, references to bringing into association will mean that the two components are rendered suitable for administration in conjunction with each other.

Thus, in relation to the process for the preparation of a kit-of-parts as hereinbefore defined, by bringing the two components ‘into association with’ each other, we include that the two components of the kit-of-parts may be:
(i) provided separately (i.e. independently of one another), which are subsequently brought together for use in conjunction with each other in combination therapy; or
(ii) packaged and presented together as separate components of a ‘combination pack’ for use in conjunction with each other in combination therapy.

Thus, there is further provided a kit of parts comprising:
(I) one of components (A) and (B) as defined herein; together with
(II) instructions to use that component in conjunction with the other of the two components.

In relation to kits of parts described above, although the compound of the invention may be provided in the form of an (e.g. pharmaceutical) formulation, in admixture with one or more additional pharmaceutically-acceptable excipients (e.g. adjuvants, diluents or carriers), when the compound of the invention is provided with a view to it primarily performing its function as a medical device or as an excipient, it may not be provided along with such additional pharmaceutically-acceptable excipients. In any event, it is preferred that the (other) pharmaceutically-active ingredient of the kit of parts is provided in the form of a pharmaceutical formulation in admixture with a pharmaceutically-acceptable adjuvant, diluent or carrier.

The kits of parts described herein may comprise more than one (e.g. formulation including an) appropriate quantity/dose of a compound of the invention, and/or more than one (e.g. formulation including an) appropriate quantity/dose of the other pharmaceutically-active ingredient, in order to provide for repeat dosing. If more than one formulation comprising or quantity/dose of either of the foregoing is present, such may be the same, or may be different in terms of the dose of either compound, chemical composition(s) and/or physical form(s).

With respect to the kits of parts as described herein, by ‘administration in conjunction with’, we include that respective components are administered, sequentially, separately and/or simultaneously, over the course of treatment of the relevant condition.

Thus, in respect of the combination product according to the invention, the term ‘administration in conjunction with’ includes that the two components of the combination product (compound of the invention and other pharmaceutically-active ingredient) are administered (optionally repeatedly), either together, or sufficiently closely in time, to enable a beneficial effect for the patient, that is greater, over the course of the treatment of the relevant condition, than if either the compound of the invention, or (e.g. a formulation comprising) the other agent, are administered (optionally repeatedly) alone, in the absence of the other component, over the same course of treatment. Determination of whether a combination provides a greater beneficial effect in respect of, and over the course of treatment of, a particular condition will depend upon the condition to be treated or prevented, but may be achieved routinely by the skilled person.

Further, in the context of a kit of parts according to the invention, the term ‘in conjunction with’ includes that one or other of the two components may be administered (optionally repeatedly) prior to, after, and/or at the same time as, administration of the other component. When used in this context, the terms ‘administered simultaneously’ and ‘administered at the same time as’ include that individual quantities/doses of the relevant compound of the invention and other active pharmaceutical ingredient are administered within 48 hours (e.g. 24 hours) of each other.

In relation to combined preparations and kits of parts described above, it is preferred that the other pharmaceutically-active ingredient is an anti-inflammatory agent, or agent known to give rise to inflammation as a side-effect, as hereinbefore described.

Wherever the word ‘about’ is employed herein, for example in the context of amounts, such as concentrations and/or doses of active ingredients and/or compounds of the invention, molecular weights or pHs, it will be appreciated that such variables are approximate and as such may vary by ±10%, for example ±5% and preferably ±2% (e.g. ±1%) from the numbers specified herein. In this respect, the term ‘about 10%’ means e.g. ±10% about the number 10, i.e. between 9% and 11%.

Compounds of the invention have the advantage that they have a wide variety of uses, including:
as biologically-active agents in variety of conditions characterised by inflammation, whether that condition is an organic inflammatory disease per se or is associated with, or is characterised by, inflammation (e.g. a wound or a burn), and/or in surgical and/or cosmetic applications as described hereinbefore
in combination with active pharmaceutical ingredients, either in combination therapy, or by performing a more inert function either as, or as part of:
   a pharmaceutically-acceptable excipient (e.g. an adjuvant, diluent or carrier),
   a medical device, and/or
   the medical device part of a drug-medical device combination.

The compounds, uses and methods described herein may also have the advantage that, in the treatment of the conditions mentioned hereinbefore, they may be more convenient for the physician and/or patient than, be more efficacious than, be less toxic than, have a broader range of activity than, be more potent than, produce fewer side effects than, or that it/they may have other useful pharmacological properties over, similar compounds or methods (treatments) known in the prior art, whether for use in the treatment of inflammation, inflammatory disorders, or disorders characterised by inflammation as a symptom (including wounds), or otherwise.

The invention is illustrated by the following examples.

### EXAMPLES

### Example 1

(SEQ ID NO: 40)
(Ala-Lys-Pro-Ser-Tyr-Hyp-Hyp-Thr-DOPA-Lys)₂

Fmoc-Lys(Boc)-Wang resin (9.15 g, GLS180322-41301, GL Biochem, Shanghai, China) was loaded into a glass reaction column.

Methylene chloride (DCM, 200 mL; Shandong Jinling Chemical Industry Co. Ltd., Shandong, China) was added to the column and allowed to soak the resin for about half an hour. The DCM was then removed by vacuum filtration.

The resin was washed 3 times with N,N-dimethylformamide (DMF, 200 mL; Shandong Shitaifeng Fertilizer Industry Co. Ltd., Shandong, China).

A 20% piperidine solution in DMF (200 mL; Shandong Shitaifeng Fertilizer Industry Co. Ltd., Shandong, China) and was added as deprotection solution and reacted for 20 minutes. The solution was then removed by vacuum filtration and the column was washed with DMF six times.

Fmoc-DOPA(Acetonide)-OH (4.14 g; GLS190219-21003, GL Biochem, Shanghai, China) and 2-(1H-benzotriazole-1-yl)-1,1,3,3-tetramethylaminium tetrafluoroborate (TBTU, 2.89 g; GLS170805-00705, GL Biochem, Shanghai, China) were added to the resin. DMF (150 mL) was added to the reaction column, followed by N,N-diisopropylethylamine (DIPEA, 2.33 g; Suzhou Highfine Biotech Co. Ltd., Jiangsu, China). A Kaiser Test was carried out with few of the resin after 30 minutes reaction, a yellow color of the solution and colorless gel indicated that the reaction was complete. The solvent was removed by vacuum filtration.

The above coupling steps were repeated to couple the remaining amino acids in the same amounts (by mols): Fmoc-Thr(tBu)-OH, Fmoc-4-Hyp(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH.

After the Fmoc-Ala-OH was coupled to the resin, the above coupling steps were repeated starting with Fmoc-Lys(Boc)-OH and followed by Fmoc-DOPA(Acetonide)-OH, Fmoc-Thr(tBu)-OH, Fmoc-4-Hyp(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH.

In a separate procedure, after Fmoc-Ala-OH was coupled on the resin, a deprotection step was carried out to remove the Fmoc protection on Dopa. The resin was washed 3 times with DMF (200 mL each time). A 20% piperidine solution in DMF (200 mL) was added as a deprotection solution and reacted for 20 minutes. Then, the resin was washed three times each with the following solvents, DMF (200 mL each time), DCM (200 mL each time) and methanol (200 mL each time; Xilong Scientific Co., Ltd., Guangdong, China). The resin was dried under vacuum for about 2 hours.

160.0 mL (i.e. 10 mL per gram of the dried resin) of lysate, which comprised of 95% trifluoroacetic acid (TFA), 2.5% water and 2.5% triisopropylsilane (Tis), were added to immerse the resin-bounded peptide-containing compound. After cleavage for about 2 hours, the solid support was removed by filtration and the filtrate was collected under reduced pressure. The filtrate was precipitated with 1600 mL (i.e. 10 mL per ml of the filtrate) of diethyl ether (Xilong Scientific Co. Ltd., Guangdong, China) and the sediment was collected by filtration. The sediment was dried by vacuum for about 2 hours, yielding 7.53 g of the crude title compound.

The crude product was firstly analyzed as a 1 mg/mL sample in pure water and detected using a Shimadzu LCMS-8050 system. The analysis column was an Agilent ZORBAX Eclipse SB-C18 (4.6×250 mm, 5 μm column; detection: UV at 220 nm; solvent A: 0.1% TFA in MeCN, solvent B: 0.1% TFA in water, with a linear gradient from 5%-90% solvent A concentration in 50 minutes; flow rate 1.0 mL/min; sample volume: 10 μL).

The target peak was eluted at 11.926 minutes and had the expected molecular weight (MS: m/z 2380.6), with a purity of 60.345%.

7.5 g of the crude product was then dissolved in 80 mL of pure water and purified using LC3000 semi-preparation equipment. The preparation column model was a Dubhe-C18 model (Hanbon Sci. & Tech. Co. Ltd., Jiangsu, China) (50*250 mm, 100 Å column; detection: UV at 220 nm). The appropriate gradient for elution was calculated from LCMS detection step (Solvent A: 0.1% TFA in MeCN, solvent B: 0.1% TFA in water, with a linear gradient from 5%-20% solvent A concentration in 30 minutes; flow rate 60.0 mL/min). Fractions were collected and analyzed using a Shimadzu LC-20 HPLC system (column as above, except with a linear gradient from 5%-30% solvent A concentration in 25 minutes).

Fractions with a purity of 98% were then mixed for an anion exchange step. This was achieved using a LC3000 semi-preparation equipment (preparation column model: Dubhe-C18 model (as above). The fractions were diluted one time with pure water and loaded to the column directly, after that the column was washed with 0.37% of ammonium acetate in pure water for about 20 minutes followed by pure water for another 20 minutes at the flow rate of 60 mL/min, then eluted with the following gradient (Solvent A: 0.1% HAc in MeCN, solvent B: 0.1% HAc in water, with a linear gradient from 5%-20% solvent A concentration in 30 minutes; flow rate 60.0 mL/min). The fractions were collected and analyzed using Shimadzu LC-20 HPLC system (column and conditions as above). Fractions with a purity of 98% were mixed and freeze-dried to give 3.06 g of the purified title compound.

### Example 2

(SEQ ID Nos: 41, 42 and 43)
(Ala-Lys-Pro-Ser-Tyr-Hyp-Hyp-Thr-DOPA-Lys)_{3~5}

One or more of the procedures described in Example 1 were repeated. Once the amino acids were coupled to the resin, the procedure was repeated three more times as described in Example 1 to provide (Ala-Lys-Pro-Ser-Tyr-Hyp-Hyp-Thr-DOPA-Lys)s (SEQ ID No: 43).

The five decapeptide repeat product peaks were detected at 13.511 minutes by LCMS (analysis column model: GS-120-5-C18-BIO, 4.6*250 mm; detection: UV at 220 nm; solvent A: 0.1% TFA in MeCN, solvent A: 0.1% TFA in water; gradient: 0˜25 min, 5%˜30% B; flow rate 1.0 mL/min.; volume: 10 μL) and the compound was isolated.

MS (five decapeptide repeat product): m/z 5924.6

To provide (Ala-Lys-Pro-Ser-Tyr-Hyp-Hyp-Thr-DOPA-Lys)₃ (SEQ ID No: 41) and (Ala-Lys-Pro-Ser-Tyr-Hyp-Hyp-Thr-DOPA-Lys)₄ (SEQ ID No: 42), the procedure as described in Example 1 is repeated once or twice, as necessary.

MS (three decapeptide repeat product): m/z is 3562.0
MS (four decapeptide repeat product): m/z is 4743.3

### Example 3

(SEQ ID NO: 45)
(Ala-Lys-Pro-Ser-Tyr-Hyp-Hyp-Thr-DOPA-Lys)₂-Lys

Fmoc-Lys(fmoc)-Wang resin (9.9 g, GLS191010-41303, GL Biochem, Shanghai, China) was loaded into a glass reaction column.

The method was the same as the first one described in Example 1 above, except that Fmoc-Lys(Boc)-OH was coupled to the resin first followed by Fmoc-Dopa(Acetonide)-OH, Fmoc-Thr(tBu)-OH, Fmoc-4-Hyp(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH, and the amounts of the amino acids, TBTU and DIPEA were doubled (by mols) compared to Example 1.

Repeating essentially the same procedure gave a further batch of crude title compound (yield 7.89 g). Analysis showed a target peak that was eluted at 11.376 minutes with the expected molecular weight (MS: m/z 2508.8). The purity was 68.985%.

7.8 g of the crude product was then purified as described in Example 1 above to give 2.57 g of pure title compound after freeze-drying.

### Example 3

(SEQ ID NO: 45)
(Ala-Lys-Pro-Ser-Tyr-Hyp-Hyp-Thr-DOPA-Lys)₂-Lys

The method is the same as described in Example 3, except the resin used is either Fmoc-Orn(fmoc)-Wang resin or Fmoc-Dap(fmoc)-Wang resin.

### Example 5

(SEQ ID NO: 48)
[(Ala-Lys-Pro-Ser-Tyr-Hyp-Hyp-Thr-DOPA-Lys)₂-
Lys]₂-Lys

The method was the same as described in Example 3, starting with Fmoc-Lys(fmoc)-OH followed by Fmoc-Lys(boc)-OH, Fmoc-Dopa(Acetonide)-OH, Fmoc-Thr(tBu)-OH, Fmoc-4-Hyp(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH, and the amounts of the amino acids, TBTU and DIPEA were doubled (by mol) compared to Example 3.

Repeating essentially the same procedure gave a further batch of crude title compound (yield 15.29 g). Analysis showed a target peak that was eluted at 11.563 minutes with the expected molecular weight (MS: m/z 5127.62). The purity was 52.126%.

15.2 g of the crude product was then purified as described in Example 1 above to give 4.96 g of pure title compound after freeze-drying.

### Example 6

(SEQ ID Nos: 49 and 50)
[(Ala-Lys-Pro-Ser-Tyr-Hyp-Hyp-Thr-DOPA-Lys)₂-
Orn]₂-Orn
or
-Dap]₂-Dap

The method is the same as described in Example 5, except using Fmoc-Orn(fmoc)-Wang resin or Fmoc-Dap acid(fmoc)-Wang resin instead. The first amino acid coupled to the resins are Fmoc-Orn(fmoc)-OH or Fmoc-Dap(fmoc)-OH, as appropriate, instead of Fmoc-Lys(fmoc)-OH.

### Example 7

(SEQ ID NO: 51)
{[(Ala-Lys-Pro-Ser-Tyr-Hyp-Hyp-Thr-DOPA-Lys)₂-
Lys]₂-Lys}₂-Lys

The method was the same as described in Example 5, except the first amino acid coupled to the resin was Fmoc-Lys(fmoc)-OH, followed by Fmoc-Lys(fmoc)-OH and then Fmoc-Lys(boc)-OH, Fmoc-DOPA(Acetonide)-OH, Fmoc-Thr(tBu)-OH, Fmoc-4-Hyp(tBu)-OH, Fmoc-4-Hyp(tBu)-OH, Fmoc-Tyr(tBu)-OH, Fmoc-Ser(tBu)-OH, Fmoc-Pro-OH, Fmoc-Lys(Boc)-OH and Fmoc-Ala-OH, and the amounts of the amino acids, TBTU and DIPEA were doubled (by mol) compared to Example 5.

Repeating essentially the same procedure gave a further batch of crude title compound (yield 28.14 g). Analysis showed a target peak that was eluted at 11.753 minutes with the expected molecular weight (MS: m/z 10365.2). The purity was 30.423%.

28.1 g of the crude product was then purified as described in Example 1 above to give 5.72 g of pure title compound after freeze-drying.

The compound of Example 7 is referred to hereinafter as ‘Compound B’.

### Example 8

(SEQ ID NO: 52)
{[(Dopa-Ala-Lys-Pro-Ser-Tyr-Hyp-Hyp-
Thr-Tyr-Lys)₂-Lys]₂-Lys}₂-Lys

The method was the same as described in Example 7, except the fourth amino acid coupled to the resin was Fmoc-Tyr(tBu)-OH instead of Fmoc-DOPA(Acetonide)-OH, and after Fmoc-Ala-OH could to the resin in the end, one more amino acid Fmoc-DOPA(Acetonide)-OH was coupled to the resin.

### MS: m/z 11671.1

The compound of Example 8 is referred to hereinafter as ‘Compound A’.

### Example 8

(SEQ ID NO: 52)
{[(Dopa-Ala-Lys-Pro-Ser-Tyr-Hyp-Hyp-
Thr-Tyr-Lys)₂-Lys]₂-Lys}₂-Lys

The method is the same as described in Example 7, except using Fmoc-Orn(fmoc)-Wang resin or Fmoc-Dap acid(fmoc)-Wang resin instead. The first two amino acids coupled to the resins are Fmoc-Orn(fmoc)-OH or Fmoc-Dap(fmoc)-OH, as appropriate, instead of Fmoc-Lys(fmoc)-OH.

### Example 10

### Mixture of (Ala-Lys-Pro-Ser-Tyr-Hyp-Hyp-Thr-DOPA-Lys)₂ (SEQ ID No: 40) Self-Crosslinked Products

4 mg of the product of Example 1 (Ala-Lys-Pro-Ser-Tyr-Hyp-Hyp-Thr-DOPA-Lys)₂ (SEQ ID No: 40) and 0.33 mg of Mushroom Tyrosinase (Sigma: T3824-250KU, 2687 units/mg) were added to 2.2 mL of a phosphate buffer solution (100 mM, pH 6.5) containing 25 mM ascorbic acid. The mixture was stirred for 2 hours. 0.15 mL of 1M HCl solution was then added to the mixture to stop the reaction.

Samples were taken for MALDI-TOF mass spectrum analysis. The results reveal that the molecular weight of the two repeat linear peptide (Ala-Lys-Pro-Ser-Tyr-Hyp-Hyp-Thr-DOPA-Lys)₂ could increase 2 to 6 times than its original molecular weight.

### Example 11

### Synthesis of Further Two-Pronged Branched Peptides

The following peptides were synthesised essentially the same processes as those described in Example 3 above, except that appropriate amino acids were used in the appropriate peptide coupling sequences:

(SEQ ID No: 55)
(Ala-Lys-Pro-Ser-Tyr-Hyp-Hyp-
Thr-Tyr-Lys)₂-Lys;
(SEQ ID No: 56)
(Ala-Lys-Pro-Ser-Tyr-Hyp-Thr-
Tyr-Hyp-Lys)₂-Lys;
(SEQ ID No: 57)
(Ala-Lys-Pro-Ser-Tyr-Hyp-Thr-
DOPA-Hyp-Lys)₂-Lys;
(SEQ ID No: 58)
(DOPA-Ala-Lys-Pro-Ser-Tyr-Hyp-
Thr-Tyr-Hyp-Lys)₂-Lys;
(SEQ ID No: 59)
(Ala-Lys-Pro-Ser-DOPA-Hyp-Thr
-DOPA-Hyp-Lys)₂-Lys;
and
(SEQ ID NO: 60)
(DOPA-Ala-Lys-Pro-Ser-DOPA-Hyp-
Thr-DOPA-Hyp-Lys)₂-Lys.

The crude yields and purity, retention time, MS values and final yields from these peptide syntheses were as shown in Table 1 below.

TABLE 1

| SEQ ID | Crude | Crude | Retention | | |
|---|---|---|---|---|---|
| No. | amount | purity | time | MS | Final amount |
| 55 | 7.59 g | 67.392% | 11.395 | 2476.8 | 2.47 g |
| 56 | 7.38 g | 69.176% | 10.957 | 2476.5 | 2.34 g |
| 57 | 7.86 g | 68.319% | 11.493 | 2508.9 | 2.51 g |
| 58 | 7.94 g | 65.843% | 11.764 | 2835.0 | 2.57 g |
| 59 | 7.63 g | 66.125% | 11.356 | 2540.3 | 2.39 g |
| 60 | 8.05 g | 64.934% | 11.798 | 2899.1 | 2.46 g |

### Example 12

### Synthesis of Further Four-Pronged Branched Peptides

The following peptides were synthesised essentially the same processes as those described in Example 5 above, except that appropriate amino acids were used in the appropriate peptide coupling sequences:

(SEQ ID No: 61)
[(Ala-Lys-Pro-Ser-Tyr-Hyp-Hyp-Thr-
Tyr-Lys)₂-Lys]₂-Lys;
(SEQ ID No: 62)
[(Ala-Lys-Pro-Ser-Tyr-Hyp-Thr-Tyr-
Hyp-Lys)₂-Lys]₂-Lys;
(SEQ ID No: 63)
[(Ala-Lys-Pro-Ser-Tyr-Hyp-Thr-DOPA-
Hyp-Lys)₂-Lys]₂-Lys;
(SEQ ID No: 64)
[(DOPA-Ala-Lys-Pro-Ser-Tyr-Hyp-Thr-
Tyr-Hyp-Lys)₂-Lys]₂-Lys;
(SEQ ID No: 65)
[(Ala-Lys-Pro-Ser-DOPA-Hyp-Thr-DOPA-
Hyp-Lys)₂-Lys]₂-Lys;

hereinafter ‘Compound C’); and

(SEQ ID NO: 66)
[(DOPA-Ala-Lys-Pro-Ser-DOPA-Hyp-
Thr-DOPA-Hyp-Lys)₂-Lys]₂-Lys

The crude yields and purity, retention time, MS values and final yields from these peptide syntheses were as shown in Table 2 below.

TABLE 2

| SEQ ID | Crude | Crude | Retention | | |
|---|---|---|---|---|---|
| No. | amount | purity | time | MS | Final amount |
| 61 | 15.06 g | 51.071% | 11.697 | 5063.2 | 4.68 g |
| 62 | 15.21 g | 50.944% | 11.684 | 5063.0 | 4.59 g |
| 63 | 15.19 g | 52.194% | 11.589 | 5127.2 | 4.46 g |
| 64 | 15.32 g | 50.058% | 11.729 | 5780.3 | 4.62 g |
| 65 | 15.25 g | 52.165% | 11.536 | 5191.5 | 4.33 g |
| 66 | 15.53 g | 50.137% | 11.785 | 5904.4 | 4.78 g |

### Example 13

### Synthesis of Further Eight-Pronged Branched Peptides

The following peptides were synthesised essentially the same processes as those described in Example 7 above, except that appropriate amino acids were used in the appropriate peptide coupling sequences:

(SEQ ID No: 67)
{[(Ala-Lys-Pro-Ser-Tyr-Hyp-Hyp-Thr-
Tyr-Lys)₂-Lys]₂-Lys}₂-Lys;
(SEQ ID No: 68)
{[(Ala-Lys-Pro-Ser-Tyr-Hyp-Thr-Tyr-
Hyp-Lys)₂-Lys]₂-Lys}₂-Lys;
(SEQ ID No: 69)
{[(Ala-Lys-Pro-Ser-Tyr-Hyp-Thr-DOPA-
Hyp-Lys)₂-Lys]₂-Lys}₂-Lys;
(SEQ ID No: 70)
{[(DOPA-Ala-Lys-Pro-Ser-Tyr-Hyp-Thr-
Tyr-Hyp-Lys)₂-Lys]₂-Lys}₂-Lys;
(SEQ ID No: 71)
{[(Ala-Lys-Pro-Ser-DOPA-Hyp-Thr-DOPA-
Hyp-Lys)₂-Lys]₂-Lys}₂-Lys;
and
(SEQ ID No: 72)
{[(DOPA-Ala-Lys-Pro-Ser-DOPA-Hyp-Thr-
DOPA-Hyp-Lys)₂-Lys]₂-Lys}₂-Lys.

The crude yields and purity, retention time, MS values and final yields from these peptide syntheses were as shown in Table 3 below.

TABLE 3

| SEQ ID | Crude | Crude | Retention | | |
|---|---|---|---|---|---|
| No. | amount | purity | time | MS | Final amount |
| 67 | 28.04 g | 31.129% | 11.798 | 10237.2 | 5.87 g |
| 68 | 27.79 g | 30.586% | 11.706 | 10237.5 | 5.58 g |
| 69 | 27.83 g | 30.638% | 11.693 | 10365.5 | 5.63 g |
| 70 | 28.87 g | 29.398% | 11.895 | 11671.1 | 5.78 g |
| 71 | 29.06 g | 30.234% | 11.957 | 10493.3 | 5.94 g |
| 72 | 28.04 g | 28.957% | 11.984 | 11927.1 | 5.66 g |

### Example 14

(SEQ ID NO: 73)
(HCA-Ala-Lys-Pro-Ser-Tyr-Hyp-
Thr-Tyr-Hyp-Lys)₂-Lys

The title compound was prepared using essentially the same process as described in Example 3 above, except that a final coupling with 3,4-dihydroxyhydrocinnamic acid (3.28 g, Macklin, Shanghai, China) was performed to yield 7.78 g of crude title compound.

Analysis showed a target peak that was eluted at 10.684 minutes with the expected molecular weight (MS: m/z 2805.0). The purity was 62.283%.

7.7 g of the crude product was then purified as described in Example 1 above to give 2.46 g of pure title compound after freeze-drying.

### Example 15

(SEQ ID NO: 74)
[(HCA-Ala-Lys-Pro-Ser-Tyr-Hyp-Thr-
Tyr-Hyp-Lys)₂-Lys]₂-Lys

The title compound was prepared using essentially the same process as described in Example 5 above, except that a final coupling with 3,4-dihydroxyhydrocinnamic acid (6.56 g) was performed to yield 15.77 g of crude title compound.

Analysis showed a target peak that was eluted at 10.727 minutes with the expected molecular weight (MS: m/z 5720.1). The purity was 48.274%.

15.7 g of the crude product was then purified as described in Example 1 above to give 4.59 g of pure title compound after freeze-drying.

### Example 16

(SEQ ID NO: 75)
{[(HCA-Ala-Lys-Pro-Ser-Tyr-Hyp-Thr-Tyr-
Hyp-Lys)₂-Lys]₂-Lys}₂-Lys

The title compound was prepared using essentially the same process as described in Example 7 above, except that a final coupling with 3,4-dihydroxyhydrocinnamic acid (13.12 g) was performed to yield 28.69 g of crude title compound.

Analysis showed a target peak that was eluted at 10.833 minutes with the expected molecular weight (MS: m/z 11551.1). The purity was 28.821%.

28.6 g of the crude product was then purified as described in Example 1 above to give 5.62 g of pure title compound after freeze-drying.

### Example 17

(SEQ ID NO: 76)
[(Ala-Lys-Pro-Ser-DOPA-Hyp-Hyp-
Thr-DOPA-Lys)₂-Lys]₂-Lys

The title compound was prepared using essentially the same process as that described in Example 5 above, except that appropriate amino acids were used in the appropriate peptide coupling sequences, to yield 14.97 g of crude title compound.

Analysis showed a target peak that was eluted at 11.578 minutes with the expected molecular weight (MS: m/z 5191.2). The purity was 52.553%.

14.9 g of the crude product was then purified as described in Example 1 above to give 4.87 g of pure title compound after freeze-drying.

### Example 18

(SEQ ID NO: 77)
[(HCA-Ala-Lys-Pro-Ser-Tyr-Hyp-
Hyp-Thr-Tyr-Lys)₂-Lys]₂-Lys

The title compound was prepared using essentially the same process as that described in Example 15 above, except that appropriate amino acids were used in the appropriate peptide coupling sequences, to yield 15.66 g of crude title compound.

Analysis showed a target peak that was eluted at 10.697 minutes with the expected molecular weight (MS: m/z 5720.0). The purity was 49.033%.

15.6 g of the crude product was then purified as described in Example 1 above to give 4.62 g of pure title compound after freeze-drying.

### Example 19

(SEQ ID NO: 78)
(HCA-Ala-Lys-Pro-Ser-DOPA-Hyp-
Thr-DOPA-Hyp-Lys)₂-Lys

The title compound was prepared using essentially the same process as that described in Example 14 above, except that appropriate amino acids were used in the appropriate peptide coupling sequences, to yield 7.83 g of crude title compound.

Analysis showed a target peak that was eluted at 10.594 minutes with the expected molecular weight (MS: m/z 2869.1). The purity was 61.036%.

7.8 g of the crude product was then purified as described in Example 1 above to give 2.51 g of pure title compound after freeze-drying.

### Example 20

(SEQ ID NO: 79)
[(DOPA-Lys-Pro-Ser-DOPA-Hyp-Thr-
Ala-Hyp-Lys)₂-Lys]₂-Lys

The title compound was prepared using essentially the same process as that described in Example 5 above, except that appropriate amino acids were used in the appropriate peptide coupling sequences, to yield 14.67 g of crude title compound.

Analysis showed a target peak that was eluted at 11.554 minutes with the expected molecular weight (MS: m/z 5191.3). The purity was 50.576%.

14.6 g of the crude product was then purified as described in Example 1 above to give 4.64 g of pure title compound after freeze-drying.

### Example 21

(SEQ ID NO: 80)
(DOPA-Lys-Pro-Ser-DOPA-Hyp-
Hyp-Thr-Ala-Lys)₂-Lys

The title compound was prepared using essentially the same process as that described in Example 3 above, except that appropriate amino acids were used in the appropriate peptide coupling sequences to yield 7.75 g of crude title compound.

Analysis showed a target peak that was eluted at 11.059 minutes with the expected molecular weight (MS: m/z 2540.6). The purity was 65.384%.

7.7 g of the crude product was then purified as described in Example 1 above to give 2.36 g of pure title compound after freeze-drying.

### Example 22

(SEQ ID NO: 81)
(HCA-Lys-Pro-Ser-Tyr-Hyp-Hyp-Thr-Ala-Lys)₂-Lys

The title compound was prepared using essentially the same process as that described in Example 14 above, except that appropriate amino acids were used in the appropriate peptide coupling sequences, to yield 7.45 g of crude title compound.

Analysis showed a target peak that was eluted at 10.489 minutes with the expected molecular weight (MS: m/z 2446.1). The purity was 65.457%.

7.3 g of the crude product was then purified as described in Example 1 above to give 2.27 g of pure title compound after freeze-drying.

### Example 23

(SEQ ID No: 82)
[(HCA-Lys-Pro-Ser-DOPA-Hyp-Thr-Ala-Hyp-Lys)₂-Lys]₂-
Lys

The title compound was prepared using essentially the same process as that described in Example 15 above, except that appropriate amino acids were used in the appropriate peptide coupling sequences to yield 14.79 g of crude title compound.

Analysis showed a target peak that was eluted at 11.235 minutes with the expected molecular weight (MS: m/z 5067.5). The purity was 53.853%.

14.7 g of the crude product was then purified as described in Example 1 above to give 4.37 g of pure title compound after freeze-drying.

### Example 24

(SEQ ID No: 83)
[(Lys-Ala-Lys-Pro-Ser-Tyr-Hyp-Hyp-Thr-Tyr)₂-Lys]₂-
Lys

The title compound was prepared using essentially the same process as that described in Example 5 above, except that appropriate amino acids were used in the appropriate peptide coupling sequences, to yield 14.26 g of crude title compound.

Analysis showed a target peak that was eluted at 11.478 minutes with the expected molecular weight (MS: m/z 5063.3). The purity was 49.642%.

14.2 g of the crude product was then purified as described in Example 1 above to give 4.33 g of pure title compound after freeze-drying.

### Example 25

(SEQ ID No: 84)
(Lys-Ala-Lys-Pro-Ser-DOPA-Hyp-Hyp-Thr-DOPA)₂-Lys

The title compound was prepared using essentially the same process as that described in Example 3 above, except that appropriate amino acids were used in the appropriate peptide coupling sequences, to yield 7.37 g of crude title compound.

Analysis showed a target peak that was eluted at 10.672 minutes with the expected molecular weight (MS: m/z 2540.2). The purity was 61.252%.

7.3 g of the crude product was then purified as described in Example 1 above to give 2.28 g of pure title compound after freeze-drying.

### Example 26

(SEQ ID No: 85)
[(Lys-Ala-Lys-Hyp-Ser-DOPA-Hyp-Hyp-Thr-DOPA)₂-
Lys]₂-Lys

The title compound was prepared using essentially the same process as that described in Example 5 above, except that appropriate amino acids were used in the appropriate peptide coupling sequences, to yield 14.06 g of crude title compound.

Analysis showed a target peak that was eluted at 11.223 minutes with the expected molecular weight (MS: m/z 5255.3). The purity was 50.577%.

14.0 g of the crude product was then purified as described in Example 1 above to give 4.25 g of pure title compound after freeze-drying.

### Example 27

### Air Pouch Model

Healthy adult male C57BL/6 mice weighing between 20 and 30 g were supplied by Changzhou Cvens Experimental Animal Co. Ltd. Prior to any experiments being conducted, the mice were housed under standardized conditions (at a constant temperature or 22±2° C., with alternating 12-hour periods of light and darkness), and were fed on a standard mouse diet with water, for about a week. The mice were randomly divided into 9 groups as described in Table 4, with 7 mice in each group.

General anaesthesia was induced using intraperitoneal delivery of 3% chloral hydrate (Sinopharm Chemical Reagent Co., Ltd., Shanghai, China); 1 mL/10 g of body weight). The hair of the entire dorsum was shaved and depilated one day before sterile air injection.

Air pouches were produced by subcutaneous injection of sterile air (5 mL) into the intrascapular area of the mice. After three days, another injection of air (3 mL) was performed to maintain the pouches. In order to induce acute inflammation, three days after the second injection, animals received an injection of sterile carrageenan solution (CP Kelco, Taixing, Jiangsu Province, China; 1%, 0.5 mL; produced by adding 0.1 g of carrageenan powder into a beaker containing 10 mL of 0.9% saline solution and stirring). Mice were pre-treated with test samples or vehicle 1 hour before and 23 hours after the carrageenan injection into the subcutaneous air pouch. Animals were sacrificed 24 hours after the carrageenan injection.

Skin biopsies were taken from the air pouches. A part of the biopsy was fixed in formalin (produced by adding ultra-pure water to 50 mL of a 40% formaldehyde solution (Nanchang Rain Dew Experimental Equipment Co., Ltd., Nanchang, Hubei Provence, China) up to a total volume of 500 mL) and analyzed by histological embedding in paraffin wax, sectioning and staining.

The histological specimens were analyzed, and an inflammation score and an edema score were estimated. The inflammation scores were estimated by observing heamatoxylin and eosin (HE) stained slices under an optical microscope. Scores (between 1 to 3 points) were given according to the perceived inflammation level (e.g. in cases where only a small amount of inflammatory cells scattered in the area: 1 point was given (mild); in cases where many inflammatory cells were observed: 2 points were given (moderate); and, in cases with diffuse infiltration: 3 points were given (severe)). A similar scoring system was employed for edema levels (3 points for most severe and 1 point for mild) after overall observation. The scores are presented in Table 5 for each group.

After conducting some preliminary experiments to validate the model, an experiment was conducted in which mice were treated by administering test samples or vehicle in accordance with Table 4 below. Compound A and Compound B were dissolved in saline at concentrations indicated in Table 4 below (L=low dose, M=medium dose, H=high dose).

Compound A and Compound B were synthesized as described in Examples 8 and 7 above, respectively. The peptide powders were stored at −20° C. prior to use. Dexamethasone (Dex) was obtained from Shanghai Aladdin Bio-Chem Technology Co., Ltd., Shanghai, China.

TABLE 4

| Group | Treatment | Dosage (μg/mouse) |
|---|---|---|
| Control | Untreated mice | / |
| Model | Model + saline | / |
| Dex | Model + dexamethasone | 400 |
| A- L | Model + Compound A | 50 |
| A-M | Model + Compound A | 250 |
| A-H | Model + Compound A | 1250 |
| B-L | Model + Compound B | 50 |
| B-M | Model + Compound B | 250 |
| B-H | Model + Compound B | 1250 |

The histological specimens were analyzed and scored as outlined above, as the results are shown in Table 5 below.

TABLE 5

| Group | Inflammation score | Edema score | Sum of the scores |
|---|---|---|---|
| Control | 0.00 | 1.14 | 1.14 |
| Model | 2.30 | 2.71 | 5 |
| Dex | 0.57 | 1.57 | 2.14 |
| A- L | 1.86 | 1.71 | 3.57 |
| A-M | 1.57 | 1.57 | 3.14 |
| A-H | 1.27 | 1.71 | 2.98 |
| B-L | 1.83 | 2.00 | 3.83 |
| B-M | 1.80 | 2.14 | 3.94 |
| B-H | 1.56 | 1.57 | 3.13 |

The histological analysis results show that Compound A and Compound B had some anti-inflammatory effects in comparison to the model group.

### Example 28

### Acute Wound Model

6-8 weeks old male C57BL/6 mice were supplied by Changzhou Cvens Experimental Animal Co. Ltd. (Changzhou, Jiangsu Province, China). Prior to any experiments being conducted, mice were housed under standardized conditions at a constant temperature of 22±2° C., with alternating 12-hour periods of light and darkness, and were fed on a standard mouse diet with water, for about a week.

General anesthesia was induced using intraperitoneal administration of 3% chloral hydrate (1 mL/10 g of body weight). The hair on the back was shaved by a baby hair shaver and depilated with cream. The skin area was wiped and sterilized with 75% alcohol twice.

A 12 mm EMS skin biopsy punch (Electron Microscopy Sciences, P.O. Box 550, 1560 Industry Road, Hatfield, PA 19440) was used to make two round wounds on the midline of the back. The two wounds were tangential and the skin between the circles was cut along the upper and lower tangents. Scissors were used to trim the wound. The full thickness of skin was removed to reach the fascia. The wound was oval shape and was left open without suture.

Different drugs as below were administrated topically at 50 μL/wound, once daily from Day 0 to Day 7 (see Table 6 below). The control group did not have a wound inflicted. The model group received the same amount of normal saline. There were 10 mice in each group apart from the control group, which had 5 mice.

Recombinant human epidermal growth factor (rhEGF, Shanghai Haohai Biological Technology Co. Ltd., Shanghai, China) was purchased and prepared according to the instructions by the manufacturer instructions. Lyophilized rhEGF powder (100000 IU/vial) was dissolved in 20 mL of normal saline to make a solution with a 5000 IU/mL concentration. The dose of rhEGF for this experiment was 1285 IU/wound. Compound A and Compound B were dissolved in saline at concentrations indicated in Table 6 (L=low dose, M=medium dose, H=high dose).

TABLE 6

| Group | Amount drug (μg/wound/day) |
|---|---|
| Control | / |
| Model | / |
| EGF | 1285 IU (500 IU/cm²) |
| A- L | 1.65 |
| A-M | 16.45 |
| A-H | 164.5 |
| B-L | 1.65 |
| B-M | 16.45 |
| B-H | 164.5 |

Photographs were taken of each wound every other day from Day 0. Photos were scanned into a computer, and wound areas calculated using ImageJ image analysis software (National Institute of Health).

The unhealed wound area was expressed as a percentage of the original wound area:

Aₜ/A₀×100%,

where A₀ and Aₜ refer to the initial area at Day 0 and the wound area at the date of measurement (time t), respectively.

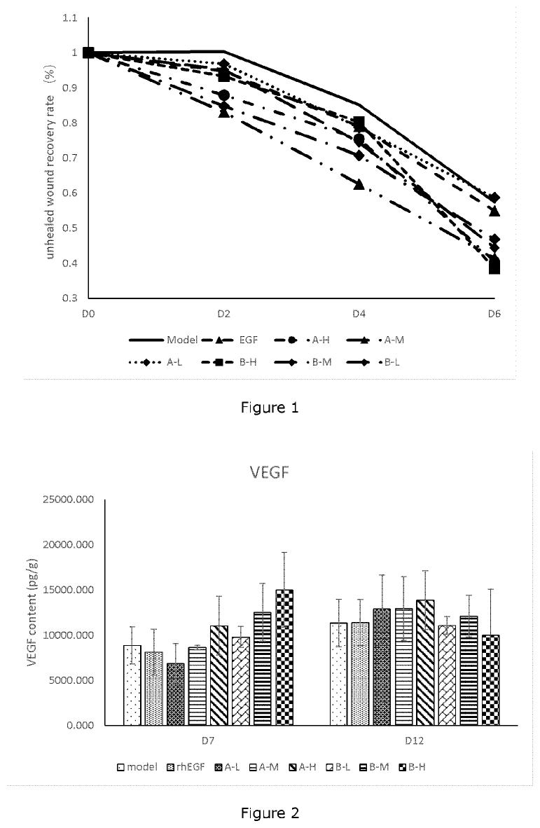

The unhealed wound rate is shown in FIG. 1. All of the wounds in the testing groups were healed faster than that in the model group. Compound A and Compound B appeared to significantly improve wound healing, especially at medium concentration of Compound A and high concentration of Compound B.

### Example 29

### Diabetic Wound Model

8 to 12 week old male db/db mice (C57BL/KsJ-db/db, with a body weight of 35-45 g/mouse) supplied by Changzhou Cvens Experimental Animal Co. Ltd. Prior to any experiments being conducted, mice were housed under standardized conditions at a constant temperature of 22±2° C., with alternating 12-hour periods of light and darkness, and were fed on a standard mouse diet with water, for about a week.

General anesthesia was induced using intraperitoneal 3% chloral hydrate (Sinopharm Chemical Reagent Co., Ltd.; 1 mL/10 g of body weight). The hair on the back was shaved by a baby hair shaver and depilated with cream. The skin area was wiped and sterilized with 75% alcohol twice.

An EMS skin biopsy punch with an 18 mm diameter was used to make a round wound on the back. The full thickness of skin was removed, and the depth of the wound reached the fascia. Wounds were left open without a suture.

Different drugs were administrated topically at 50 μL/wound, once daily from Day 0 to Day 18, as shown in Table 7 below. The control group did not have wound inflicted.

The model group was given same amount of normal saline. There were 12 mice in each group apart from the control group, which had 8 mice. The skin pieces taken during wound creation were used as the samples at Day 7 for the control group.

Recombinant Human Epidermal Growth Factor (rhEGF) was purchased and prepared according to the instructions by the manufacturer. The lyophilized rhEGF powder (100000 IU/vial) was dissolved in 20 mL of normal saline to make a solution with a concentration of 5000 IU/mL. The working dose of rhEGF for this experiment was 1285 IU/wound.

Compound A and Compound B were dissolved in saline at concentrations indicated in Table 7 below (L=low dose, M=medium dose, H=high dose). 50 μL of each solution was applied to the wound surfaces every day.

TABLE 7

| Group | Amount drug (μg/wound/day) | |
|---|---|---|
| Control | / | |
| Model | Normal saline | |
| EGF | 1285 | IU |
| A- L | 1.65 | μg |
| A-M | 16.45 | μg |
| A-H | 164.5 | μg |
| B-L | 1.65 | μg |
| B-M | 16.45 | μg |
| B-H | 164.5 | μg |

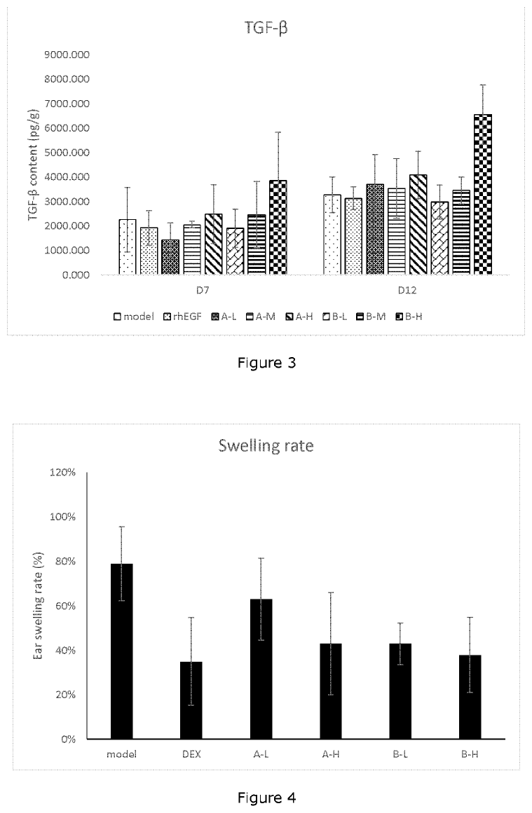

Vascular endothelial growth factor (VEGF) and transforming growth factor-beta 1 (TGF-β1) play prominent roles in wound healing process. VEGF and TGF-β1 are often co-expressed in tissues in which angiogenesis occurs. The content of these two factors in wound tissues were also detected and are shown in FIGS. 2 and 3.

The results showed that the content of VEGF and TGF-β1 in all test groups was higher than in the model group at different time, indicating that Compound A and Compound B stimulated the production of VEGF and TGF-31.

### Example 30

### Mouse Ear Swelling Model

30 Health male BALB/c mice with 6-8 weeks old and average body weight of 18-25 g were supplied by Changzhou Cvens Experimental Animal Co., Ltd. and housed and cared for about 1 week prior to the experiment. The housing temperature was around 25 to 27° C., the humidity was 74%, with alternating 12-hour periods of light and darkness, and free access to food and water. The mice were randomly divided into 6 groups as described in Table 8, with 5 mice in each group. Compound A and Compound B were dissolved at concentrations indicated in Table 8 below (L=low dose, M=medium dose, H=high dose).

TABLE 8

| Group | Drug concentration | |
|---|---|---|
| Model | / | |
| Dex cream | 10 | μg/g |
| A-L | 0.5 | mg/g |
| A-H | 1 | mg/g |
| B-L | 0.5 | mg/g |
| B-H | 1 | mg/g |

Hydrogels were prepared comprising the amounts of peptides described in Table 8, along with methyl cellulose (2.5%), propanediol (11%), glycerol (11%), pH was adjusted to 5.5 by adding acetic acid (pH regulator; 0 to 0.5 g). All excipients were obtained from Sinopharm Chemical Reagent Co. Ltd. The gels were made with water for injection.

Dexamethasone acetate cream (Dex cream; 5 mg/10 g (which means that there was 5 mg Dex contained in 10 g of the cream), Fuyuan Pharmaceutical Co., Ltd., Anhui, China) was used as positive control.

The left ear of each mouse was used as an autologous control. The right ear of each mouse was treated with the above compounds in the stated concentrations (Table 8).

About 0.1 g of the various gels, and Dex cream were applied to the right ear of mice in each group, both inside and outside. The blank gel base was applied on the ears in the model groups. After 1 hour, 20 μL of xylene (Shanghai Aladdin Bio-Chem Technology Co., Ltd.) was applied to the same ear of each mouse.

The mice were sacrificed by cervical dislocation 40 minutes after xylene application. The left and right ears were cut off. An EMS skin biopsy punch with a diameter of 8 mm was used to take a piece of the ear from the same site on both ears. The weights were recorded, and the swelling rate was calculated as a percentage according to the following formula:

(right ear weight−left ear weight)/left ear weight×100

The results are shown in Table 9 and FIG. 4.

TABLE 9

| | model | DEX | A-L | A-H | B-L | B-H |
|---|---|---|---|---|---|---|
| Swelling rate | 79% | 35% | 63% | 43% | 43% | 38% |
| SD | 0.166 | 0.198 | 0.184 | 0.231 | 0.095 | 0.17 |

The results showed that Compound A and Compound B could significantly eliminate the edema caused by inflammation.

### Example 31

### Preparation of Compound A and Compound B Coated Membranes

0.2 μm microfiltration membranes (Jinteng corp., China) were cut into 2.5 cm diameter disks and put into three different containers (each with 10-15 pieces). About 10 mL of Compound A and Compound B stock solutions with 5 mg/ml were added into the containers respectively making sure that all membranes were fully immersed. PBS buffer (pH 8.0) was added dropwise into the containers while shaking. The pH of the reaction mixtures was regularly checked until the value reached 7. The containers were carefully covered and continuously shaken for 8 hours. The reaction mixtures were then poured out and the membranes were washed with 5 mL PBS buffer each time until the washing eluents become colorless. Finally, the membranes were placed in the shade to dry. The obtained membranes coated with Compound A and Compound B were used for antioxidant tests.

The antioxidant capacity (AC) was measured using a modified DPPH (2,2-diphenyl-1-picrylhydrazyl) method (as follows). Each membrane with same weight was cut into small pieces and put in a 2 mL centrifugal tube separately. A 0.1 mM solution of DPPH in methanol was prepared and 600 μL of DPPH solution was added to make sure all the pieces were fully immersed. The tube was then kept in the dark at room temperature for 3 hours, followed by centrifugation for 5 mins. 300 μL of supernatant was added into a 96-well plate and measured at 517 nm using a microplate reader. The AC values of each membrane were calculated according to the formula shown below, where A₀ was the absorbance of DPPH solution only, and Am was the absorbance of each membrane sample.

AC(%))=(A₀−Aₘ)/A₀×100%

The antioxidant properties of the Compound A and Compound B coated membranes were tested at day 0 (DO), day 3 (D3), day 7 (D7) days and day 10 (D10) to check stability. The results are shown in Table 10.

TABLE 10

| Samples | D 0 | D 3 | D 7 | D 10 |
|---|---|---|---|---|
| Compound A coated membrane | 70.85% | 68.16% | 65.82% | 68.82% |
| Compound B coated membrane | 68.00% | 65.52% | 68.97% | 65.97% |

The results showed that both Compound A and Compound B could coat on membranes. The coated membranes had antioxidant properties and could last for at least 10 days.

### Example 32

### Croton Oil-Induced Anal Swelling Model in Rats I

A croton oil mixture was prepared by mixing one part distilled water, four parts of pyridine (Nanjing Chemical Reagent Co., Ltd.), five parts of ether (China Pharmaceutical Group Chemical Reagents Co., Ltd.) and ten parts of 6% croton oil (Shanghai Yuanye Biotechnology Co., Ltd.) ether solution.

6-8 weeks old Sprague Dawley (SD) rats with average body weights of 180-220 g were supplied by Changzhou Cvens Experimental Animal Co. Ltd. (Changzhou, Jiangsu Province, China), half male and half female. Prior to any experiments being conducted, rats were housed under standardized conditions (at a constant temperature of 22±2° C., with alternating 12-hour periods of light and darkness) and were fed on a standard mouse diet with water, for about a week.

56 rats were randomly divided into 7 groups (as shown in Table 11 below), with 8 rats in each group. Compound A, Compound B and MaYinglong hemorrhoids ointment were dissolved at concentrations indicated in Table 11 below (L=low dose, M=medium dose, H=high dose).

TABLE 11

| | | | | Volume |
|---|---|---|---|---|
| Group | Treatment | Drug concentration | | (μL) |
| Control | Normal saline | / | | 200 |
| Model | Blank gel | / | | 200 |
| MY-L | MaYinglong hemorrhoids | commercially | | 200 |
| | ointment | available Ointment | | |
| A-L | Compound A | 0.5 | mg/g | 200 |
| A-H | Compound A | 1 | mg/g | 200 |
| B-L | Compound B | 0.5 | mg/g | 200 |
| B-H | Compound B | 1 | mg/g | 200 |

The rats were anesthetized by isoflurane (China Pharmaceutical Group Chemical Reagents Co., Ltd.) inhalation. 75% alcohol cotton ball was used to disinfect the skin around the anus. Then, 0.16 mL croton oil mixture was dripped slowly on a cotton swab which was inserted 0.5 cm into the rat anus. The rat was lifted to keep the head upwards (the position was maintained for 10 seconds), then the cotton swab was withdrawn, and the croton oil mixture was evenly applied to the surrounding skin. The control group was given the same volume, but of olive oil.

One hour after modelling, rats in each group were treated according to Table 11. The positive control drug was MaYinglong hemorrhoids ointment (MaYinglong Pharmaceutical Group Co., Ltd.). The gels of Compound A and Compound B were prepared as described in Example 14. The drug was administered twice a day, once in the morning and once in the evening, for three consecutive days.

200 μL of the corresponding drugs were drawn with 1 mL syringe (needle removed). The syringe was inserted into the anal canal and about 160 mL of the respective test substance was pushed 1.5 cm into the anal canal. The remaining of the respective test substances was applied to the surrounding skin near the anus. The skin around the anus was held tightly for 1 minute to prevent drug discharge.

In the morning of the fourth day, 1% Evans blue (EB) was injected into the tail vein 30 minutes after drug administration (200 μL/100 g). The rats were sacrificed by cervical dislocation after 30 minutes.

The rats were placed in the supine position on an anatomical plate and their abdomens were opened. The rectoanal tissues (15 mm in length) were isolated and weighed and the EB dye present in the tissue was extracted using 1 mL of formamide.

All samples were transferred to a 55° C. water bath or a heat block. Incubation for 24 hours extracted EB from the tissue. The formamide/EB mixture was centrifuged to pelletize any remaining tissue fragments. Absorbance was measured at 610 nm, using 500 μL of formamide as a blank.

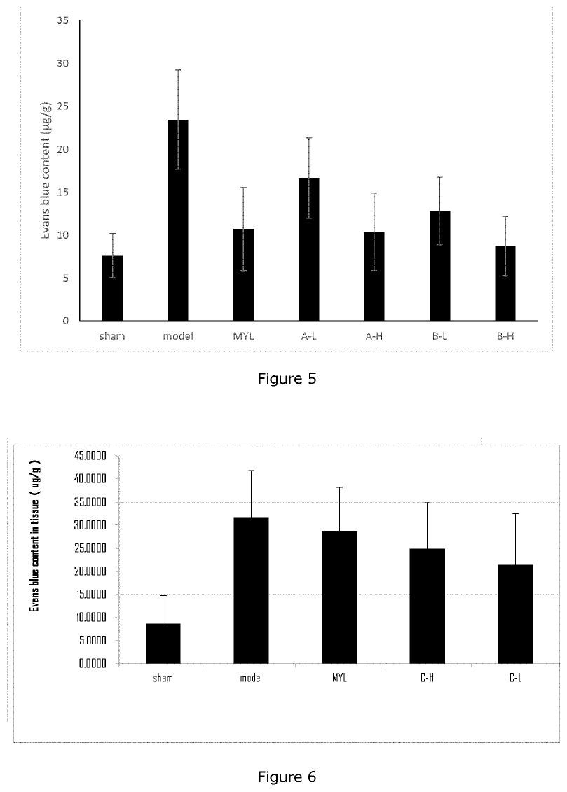

The content of EB in rectal and anal tissues was calculate using amount (in ng) of EB extravasated per mg of tissue to evaluate vascular permeability. The results are shown in FIG. 5 and show that Compound A and Compound B gel may reduce inflammatory swelling caused by croton oil application, as indicated by the variation of EB content in the different treatments. A decrease of EB concentration was an indication of vascular permeability.

### Example 33

### Radiation Proctitis

A gel comprising 0.5 g of Compound C (see Example 12 above) was made, which also consisted of the following components: methyl cellulose (2.2 g; Shandong Guangda Technology Development Co., Ltd., ShanDong, China), glycerin (11 g) and propanediol 11 g (both Sinopharm Chemical Reagent Co. Ltd.), and purified water (75.3 g).

For a 1.5 mg/g gel, the amounts of Compound C and water were adjusted accordingly.

The methyl cellulose and water were mixed together and stirred until to a homogeneous colloidal suspension was formed. Then, peptide powder, glycerin and propanediol were added to the methyl cellulose/water mixture, and the resultant mixture quickly stirred for 5 minutes to obtain the finished product.

Male Wistar rats weighing 180-220 g were obtained from Zhejiang Vital River Laboratory Animal Technology Co., Ltd. All animals were maintained on standard rodent chow and tap water in standard cages with alternating 12-hour periods of light and darkness.

The rats were anesthetized with an intraperitoneal injection of 10% chloral hydrate (3.3 mL/kg). The rats were restrained and taped by the tail and four limbs on a cardboard in supine position. Irradiation was delivered using an Elekta Synergy medical linear accelerator (Elekta limited, UK). All animals except the sham group received single continuous pelvic irradiation. The distance from animal to source was 100 cm. The radiation area was 2 cm×5 cm, 5 cm upward from the anal orifice. The radiation dose was 17.5 Gy at the dose rate of 600 cGy/min.

After irradiation, the animals were put back into the cages for natural recovery. The animals in the sham operation group (‘Sham’) were anesthetized in abdominal cavity without irradiation. Daily feed intake of rats and body weight were measured, and general observation was performed every day.

Day 1 (D1) was defined as the day of drug administration, which was 24 hours after modelling. Rats in the sham operation group and the model group (‘Model’) were given blank gel. Rats in the treatment group were given a high dose (1.5 mg/g; ‘C—H’) or a low dose (0.5 mg/g; ‘C-L’) of a rectal dressing comprising the gel (300 μL/rat), 1 time a day, and 7 days continuously (D1 to D7). In order to reduce bowel movement and to extend the duration of the gel in the rectum, all animals were given a 6 mL/kg intraperitoneal injection of 5% chloral hydrate every day before administration. Drugs were introduced into the rectum at about 6 cm by an intragastric needle. Sampling was performed at day 8 (D8). The rats were fasted for at least 12 hours in advance.

The rats were scarified by posterior carotid bloodletting after being anesthetized by an intraperitoneal injection of chloral hydrate. About 7 cm of the colorectal tract was separated at about 0.3 cm from the edge of perianal fur. The specimen was trimmed and 1 cm of proximal and distal colorectal samples were cut off, respectively, by the same person.

Then, the intestinal tube was dissected longitudinally, photographed and weighed. Specimens were fixed in 10% formaldehyde solution for 48 hours and stained with HE before examination with light microscopy by a pathologist (who was blinded to the study).

Each specimen was graded as follows: 0=normal or minor alterations which cannot be ascribed (with certainty) to radiation; 1=slight radiation damage (mild inflammation and/or slight crypt change); 2=mild damage (more significant inflammation, and/or crypt damage); 3=moderate damage (must have prominent loss of epithelium, degree of inflammation variable); and 4=severe damage (ulcers, necrosis).

Rates of body weight increase at D8 (defined as: body weight on D8−initial body weight)/initial body weight×100%) are shown in Table 12 below. A higher rate indicates a better physiological situation. Compound C was dissolved at concentrations indicated in Table 12 below (L=low dose, H=high dose).

TABLE 12

| Group | Dose | Mean (%) | Standard deviation |
|---|---|---|---|
| Sham | / | 5.7030 | 3.7042 |
| Model | / | −1.7047 | 5.2056 |
| C-L | 0.5 mg/g | 0.8076 | 5.5920 |
| C-H | 1.5 mg/g | 3.7538 | 4.0502 |

The results show that the gels comprising Compound C prevent weight loss caused by radiation proctitis, in a dose dependent manner.

### Example 34

### Radiation Vaginitis

A 45 year-old female patient diagnosed with cervical cancer was treated with radiation therapy. The radiation was delivered by high energy 6-12 MV X-ray. The irradiation dose was 1.8-2.0 Gy, 5 times a week. The radiation therapy was scheduled to be completed within 4 weeks.

Two weeks after the first irradiation, she started to feel pain, and eventually had vaginal bleeding and ulcers. This was diagnosed as radiation vaginitis.

The patient started to use 1.5 mg/g×3 g of the gel prepared as described in Example 33 above, which was packed in a special applicator for vaginal use. This was used twice a day during her radiation therapy. After 3 days, her bleeding had stopped, and the pain was reduced. After completing the radiation therapy, she continued to use the gel for a further of two weeks. Her doctor examined her vagina and found that the ulcers had disappeared, and that there was no evidence of any other damage.

### Example 35

### Croton Oil-Induced Anal Swelling Model in Rats II

Essentially the same procedure as that described in Example 32 above was carried out with the Compound C gels as described in Example 33 above on 50 rats having been randomly divided into 5 groups (with Compound C at 0.5 mg/g (Low) and 1.5 mg/g (High) doses in place of the corresponding Compound A and Compound B gels).

The content of EB in rectoanal tissues (8 mm in length) was calculated to evaluate vascular permeability as in Example 32 and the results are shown in FIG. 6. These results show that Compound C gel reduces inflammatory swelling caused by croton oil application, in a dose dependent manner.

### Example 36

### Ulcerative Proctitis Model

A total of 50 SD rats were randomly divided into 5 groups, with 10 rats in each group (sham operation (Sham); model (blank gel; Model); positive control (sulfasalazine, SSZ, 360 mg/kg; SSZ); Compound C (1.5 mg/g dose (High)); and Compound C (0.5 mg/g dose (Low) (both of which were prepared as described in Example 33 above) groups).

The animals were anesthetized with isoflurane after 24 hours of fasting. Apart from the sham operation group, the other 4 groups were perfused with 0.5 mL of 2,4,6-trinitrobenzene sulfonic acid (TNBS; 1 mL; Dalian Meilun Biotechnology Co., Ltd., China) solution in ethanol (6.05 mL; Shanghai Aladdin Biochemical Technology Co., Ltd., China) and 18.071 mL of sterilized water for injection (i.e. 18 mg TNBS/rat) through the rectum with latex hose under anesthesia.

The length of the hose entering the rectum was about 8 cm. The rats were kept in a state of isoflurane anesthesia for another 15 minutes after drawing out the hose (the day of modelling was Day 0) and then the animals were returned to their cages. The sham operation group was given the same volume of normal saline.

On the day after modelling (Day 1), gels (Model and Compound C groups) were administered rectally at a dose of 0.5 mL/rat after anesthesia (intraperitoneal injection of pentobarbital (35 mg/kg, 1.5%, 0.233 mL/100 g)). Anuses were clamped for 1 hour after administration and then released. In the positive control group, SSZ was given sulfasalazine by oral gavage. This was repeated once a day, for 7 consecutive days (Day 1 to Day 7). The sham operation group had no treatment.

The general condition, disease activity index (DIA) and body weight of rats was observed every day. On the day after final administration (Day 8), the animals were dissected, whole colon tissue was removed, and the contents of the colon cleaned. The body weight was measured and then the colon was opened longitudinally to reveal the ulcerative surface, which was measured and whose general appearance was assessed and photographed.

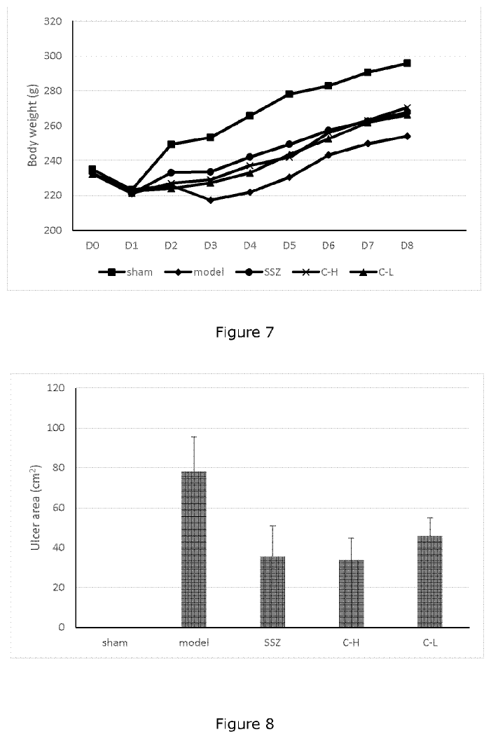

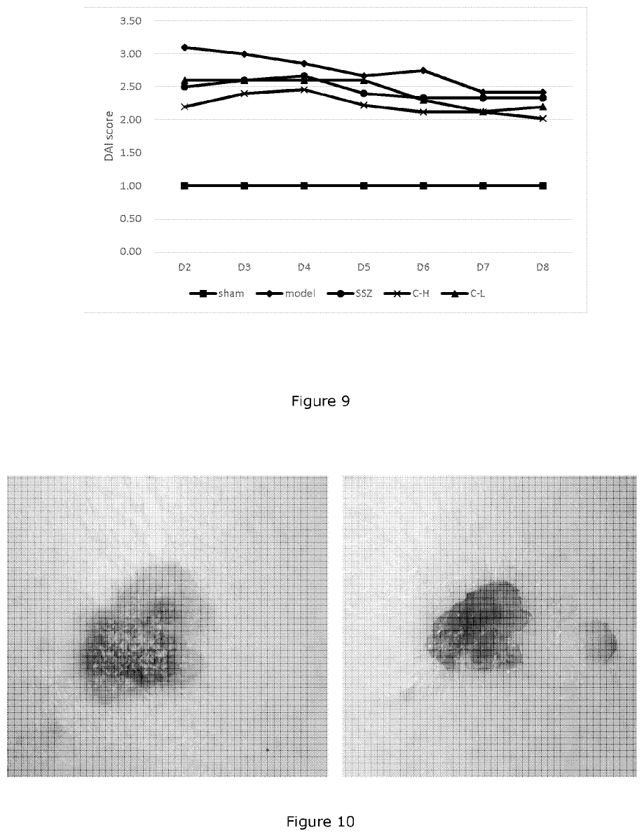

The results are shown in FIG. 7 (body weight), FIG. 8 (ulcerative surface) and FIG. 9 (general appearance), respectively, and indicated that Compound C reduces the severity of TNBS-induced ulcerative proctitis in a dose dependent manner, and may, thus, promote the healing of ulcers.

### Example 37

### Experimental Gastric Ulcer Model

SPF grade SD rats were used in this experiment, with 10 rats in each group. According to an evaluation method of health food for gastric ulcer issued by Chinese State Food and Drug Administration, the protective effect of Compound B on acute gastric ulcers induced by anhydrous alcohol was observed after continuous gavage for 30 days.

The different groups and dosages are shown in Table 13 below.

TABLE 13

| | | |
|---|---|---|
| Group | Concentration | Dose |

| | | |
|---|---|---|
| Control | / | / |
| Model | / | / |

| | | | | |
|---|---|---|---|---|
| B-L | 0.25 | mg/mL | 0.5 | mg/kg |
| B-H | 0.75 | mg/mL | 1.5 | mg/kg |
| Omeprazole | 2 | mg/mL | 4 | mg/kg |

Omeprazole enteric-coated capsules (20 mg/capsule; Heilongjiang Norgas Pharmaceutical Co., Ltd., China) were opened and the powder was dissolved in water to make a 2 mg/mL solution. Compound B (see Example 7 above) powder was dissolved in water to make two solutions with concentrations of 0.25 mg/mL and 0.75 mg/mL. Administration volumes were 2 mg/kg.

All drugs were administrated as shown in Table 13 by oral gavage for 30 consecutive days, once per day. The rats had free access to water. On the 30^{th} day, rats were fasted for 24 hours after the last drug administration. On the 31^{st} day, 1.0 mL/rat of absolute ethanol was given by gavage to each rat in all of the experimental groups except the control group.

1 hour later, all of the rats were sacrificed and dissected to expose the intact stomach, followed by ligation of the pylorus. 10% formaldehyde solution was administered into the stomach by perfusion and fixed for 20 minutes. The stomach was dissociated after fixation and cut open along the greater curvature. The stomach contents were washed off the lining with saline to reveal the gastric mucosa.

The length and width of gastric mucosal bleeding were measured with a Vernier calliper under a stereo microscope or by the naked eye. Scores were given to evaluate the damage based on the evaluation criteria showed in Table 14 below.

TABLE 14

| Damage | 1 point | 2 points | | 3 points | 4 points |
|---|---|---|---|---|---|
| Number of bleeding | 1 for each | / | | / | / |
| Width of bleeding | 1-5 mm | 6-10 | mm | 10-15 mm | >15 mm |
| Length of bleeding | 1-2 mm | >2 | mm | | |

| | |
|---|---|
| Total score | =scores of (number of bleeding + |
| | length + 2 × width) |

The results are shown in Table 15 below.

TABLE 15

| Group | Damage score |
|---|---|
| Control | 0 |
| Model | 28 |
| B-L | 24 |
| B-H | 18 |
| Omeprazole | 20 |

The results showed that Compound B may reduce gastric bleeding induced by alcohol and thus protect the gastric mucosa.

### Example 38

### Radiation Injury in the Nasal Cavity

A 50-year-old male patient was diagnosed with paranasal sinus cancer and received radiation therapy. After two weeks treatment, he had inflammation of the nose and sinuses and felt like his nose was blocked or stuffy. The mucus in the nasal cavity and paranasal sinuses became thick and dry.

The patient used a nasal spray comprising Compound B (see Example 7 above) dissolved in water at a concentration of 0.5 mg/mL and then packed into a nasal spray bottle, once every two hours during daytime. Three days later, his nose became clear and was no longer stuffy. As his radiation therapy went on, he continuously used the nasal spray, and his nasal discomfort did not worsen.

### Example 39

### Radiation Stomatitis

A 79-year-old male patient was diagnosed with oral cancer and underwent radiation therapy.

After 3 weeks, oral mucositis developed with ulcers on the mucous membrane lining the mouth, throat and esophagus. It was very painful and the patient was not able to eat.

The patient then used Compound B solution (0.5 mg/mL; prepared as described in Example 38 above) as a mouthwash, as frequently as needed. Almost immediately after application, pain was very much reduced and this relief could last for one or two hours. As his therapy continued, the patient's stomatitis did not worsen.

### Example 40

### Pain and Bleeding after an Operation

A 54-year-old man had surgery on his right leg to remove a hyperplasia of black tissue as showed in FIG. 10 (left pane). Immediately after the operation, he used 0.5 mg/mL of Compound B spray (as described in Example 38 but packed into a normal spray bottle) to apply directly on the wound.

The right pane in FIG. 10 was taken 1 hour after operation. The bleeding had stopped, and only slight pain was felt. One week later, the wound was fully recovered. The physician said that it would normally take a patient at least ten days to fully recover from such a procedure with standard care.

### Example 41

### Itch Relief

A 36-year-old woman had hemorrhoids for many years. Normally she did not feel pain or get bleeding, but has a problem with itchiness, which bothered her very much. The patient also had a problem with constipation.

The patient started to use Compound C gel (0.5 mg/g×3 g; see Example 33 above) packed into a rectal applicator, which she used once a day before bedtime.

The next day, she felt less of an itch, which disappeared after one week. At the same time, the patient's constipation became less prevalent.

### Example 42

### Ulcerative Colitis

A 39-year-old woman was diagnosed with an acute attack of ulcerative colitis. She had to go to toilet more than 10 times a day and exhibited with severe bleeding from the colon.

She was treated with oral 5-aminosalicylic acid (also known as mesalazine or mesalamine) for 3 days, but her symptoms did not change.

After this, she was given the same Compound C gel as described in Example 41 above, at the same dose, 3 times over the first two days.

The frequent with which she had to go to the toilet reduced to 3 to 4 times a day. She continuously used the gel for further 7 days, once per day, after which her symptoms disappeared.

### Example 43

### Bioadhesive

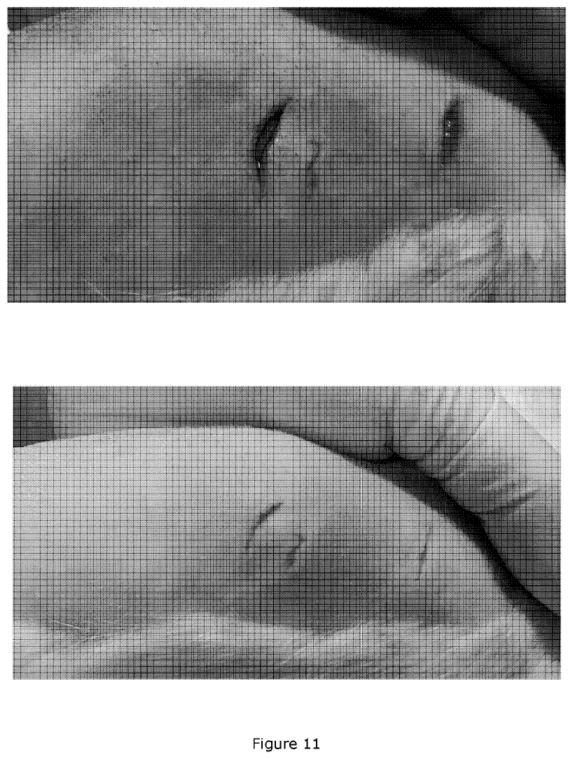

The test was carried out in a rat. Two incisions about 1 cm each were cut on the left side of a SD rat (under isoflurane inhalation anesthesia). The left incision was left untreated while the right incision was covered by a pinch of Compound C powder. Each incision was closed with tweezers for 10 seconds. The rat was then put back to the cage. After about 20 minutes, the rat woke up and started to move around. The untreated wound split but the treated wound kept closed. After 24 h, the two incisions were almost recovered with the treated wound smoother than the untreated one. The left picture (in FIG. 11) was taken immediate after the operation, while the right one was taken after 24 h.

### Example 44

### Preparation of Pre-Crosslinked Compounds of the Invention Using Glutaraldehyde I

100 mg of a peptide of any one of SEQ ID Nos: 45, 48, 51, 57, 58, 63, 64, 69 or 70 is reacted with 2-100 mL of 0.01-0.5 M buffer with different pH values (around 3.0 to 8.0) (such as, 0.01 M sodium acetate (pH 3.0), 0.1 M sodium acetate (pH 5.0), 0.2 M sodium phosphate (pH 6.0), and 0.5 M sodium phosphate (pH 8.0)), containing 0.01%-5.0% glutaraldehyde, at room temperature for 1-300 minutes. At the end of the reaction, an amount of NaHSO₃ (equivalent to 80% of the glutaraldehyde) is added to stop the reaction. The preparations are then dialyzed exhaustively against water, giving rise to the corresponding title compound(s).

### Example 45

### Preparation of a Pre-Crosslinked Compound of the Invention Using Glutaraldehyde II

Essentially the same method as that described in Example 44 above is followed to react 100 mg of [(Ala-Lys-Pro-Ser-Tyr-Hyp-Thr-DOPA-Hyp-Lys)₂-Lys]₂-Lys (SEQ ID No: 63) with 10 mL of 0.1 M sodium acetate buffer (pH 5.0), containing 0.05% glutaraldehyde, at room temperature for 10 minutes. The crosslinked extent is detected via size-exclusion chromatography (SE-HPLC).

### Example 46

### Preparation of Pre-Crosslinked Compounds of the Invention Using an Amide Formation Method I

100 mg of a peptide of any one of SEQ ID Nos: 45, 48, 51, 57, 58, 63, 64, 69 or 70 is mixed with 2-100 mL of pure water or 0.01-0.5 M buffer of different pH values (around 3.0 to 8.0) (such as, 0.01 M sodium acetate (pH 4.0), 0.05 M MES buffer (pH 5.0), 0.1 M MES buffer (pH 6.0), and 0.5 M sodium phosphate (pH 7.0)), containing 1-500 mg of each condensation agent (such as, N-(3-dimethylaminopropyl)-N′-ethylcarbodiimide hydrochloride and N-hydroxysuccinimide (EDC/NHS) or 4-(4,6-dimethoxy-1,3,5-triazin-2-yl)-4-methylmorpholinium chloride (DMTMM) or other water soluble condensation agents), at room temperature for 0.5-72 hours. At the end of the reaction, the preparations are dialyzed exhaustively against water to remove DMTMM and give rise to the corresponding title compound(s).

### Example 47

### Preparation of a Pre-Crosslinked Compound of the Invention Using an Amide Formation Method II

Using essentially the same method as that described in Example 46 above, 100 mg of [(Ala-Lys-Pro-Ser-Tyr-Hyp-Thr-DOPA-Hyp-Lys)₂-Lys]₂-Lys (SEQ ID No: 63) is reacted with 10 mL of 0.05 M MES buffer (pH 5.5), containing 100 mg of DMTMM, at room temperature for 6 hours. The crosslinked extent is detected via size-exclusion chromatography (SE-HPLC).

### Example 48

### Preparation of Pre-Crosslinked Compounds of the Invention Using a DOPA Oxidation Method I

100 mg of a peptide of any one of SEQ ID Nos: 45, 48, 51, 57, 58, 63, 64, 69 or 70, etc.) is reacted with 2-100 mL of pure water or 0.01-0.5 M buffer with different pH values (around 3.0 to 8.0) (such as, 0.01 M sodium acetate (pH 5.0), 0.05 M MES buffer (pH 5.0), 0.5 M sodium phosphate (pH 7.0), and 0.1 M Tris buffer (pH 8.0)), containing 1-500 mg of each oxidants, such as peroxide, periodate, or various phenolases (such as tyrosinase), at room temperature for 0.5-72 hours. At the end of the reaction, 0.5%-5% (v/v) of 1 M HCl solution is added to the mixture to stop the reaction, giving rise to the pre-crosslinked title compounds.

### Example 49

### Preparation of a Pre-Crosslinked Compound of the Invention Using a DOPA Oxidation Method II

Using essentially the same method as the one described in Example 48 above, 100 mg of [(Ala-Lys-Pro-Ser-Tyr-Hyp-Thr-DOPA-Hyp-Lys)₂-Lys]₂-Lys (SEQ ID No: 63) is reacted with 10 mg of Mushroom Tyrosinase (Sigma: T3824-250KU, 2687 units/mg) in 10 mL of a phosphate buffer solution (100 mM, pH 6.5). The mixture is stirred for 2 hours. 1.5 mL of 1M HCl solution is then added to the mixture to stop the reaction. Samples are taken for MALDI-TOF mass spectrum analysis. The results reveal that the molecular weight of the product increases 2 to 6 times than its original molecular weight.

### Example 50

### Effect of a Compound of the Invention on the Activity of Human Influenza A Virus H1N1

A serum free 1640 medium (RPMI1640 culture medium; GIBCO/BRL; Thermo Fisher Scientific China, Nanjing, China) was prepared according to the manufacturer's instructions. It was formulated as a complete medium containing 10% serum by adding neonatal bovine serum (Zhejiang Tianhang Biotechnology Co. Ltd., Luoshe, China) before use, or was formulated as a maintenance solution by adding 2% of the same serum.

40 mg of Compound C was dissolved in 2 mL of aqueous sodium chloride (in aqua pro injection, Jiangsu Hengrui Medicine Co., Ltd., Jiangsu Province, China) to prepare a 20 mg/mL stock solution.

0.05 mL of the stock solution was added to 1.95 mL of the complete medium to formulate a 500 μg/mL drug solution (maintenance solution was used instead of the complete medium in antivirus tests Nos. 3 and 4 below). Then, working solutions with concentrations of 250, 125, 62.5, 31.25, 15.625, 7.8125, 3.9063, 1.9531 and 0.9766 μg/mL were prepared by double dilution.

### Cytotoxicity of Compound C

Vero cells were inoculated on the 96-well culture plate and grew into monolayers. 0.2 mL of Compound C per well with different concentrations (as above) were added. This was repeated in 3 wells for each concentration. The solvent and normal cell cultures were used as a negative control. Cells were cultured at 35° C. (5% CO₂) for 24 h. 10 μl of Cell Counting Kit-8 (CCK-8, Sigma) was added into each well, mixed well and cultivated at 37° C. for 2 hours. The absorbance value of OD₄₅₀ (Optical density) was detected by enzyme-linked immunosorbent assay. The cell viability of untreated cells was set as 100%, and the cytotoxicity rate was calculated. The cytotoxicity rate (%)=(average absorbance of untreated cell−average absorbance of drug treatment well)/average absorbance of untreated cell×100%. The median lethal concentration (LC50) of the tested drug was calculated. Results showed that, under the tested concentrations, Compound C had no cytotoxicity.

Effect of Compound C on the Cytopathic Effect of Viruses after Directly Acting on H1N1

Vero cells were inoculated into 24 well plates and set aside until 70%-80% fusion degree was reached. The virus was mixed with different concentrations of the test drug. The final concentration of Compound C reached 2 μg/mL, 4 μg/mL, 8 μg/mL, 16 μg/mL, 32 μg/mL, 64 μg/mL, 128 μg/mL. 0.1% SDS (SDS; manufactured by AMRESCO LLC, Solon, OH, USA and packed by Biosharp Company, Hefei, China; purity: 99%) was used as the positive control and mixed with the virus for 1 hour. The cell culture medium was removed and the cells were washed with PBS 3 times before adding the virus/drug mixture and incubating for 1 hour. The untreated virus was used as the negative control. After the 1-hour incubation, the culture was washed with PBS 3 times, and continually cultured for 16-24 hours. RNA was extracted, and fluorescence quantitative test was performed with one step QRT PCR kit to detect the antiviral effect.

The virus inhibition rate was calculated. Cells in the non-drug challenge group was set as with 0%. The virus inhibition rate (%)=(1-drug treatment group viral RNA %)×100%. The IC50 of 50% effective concentration was calculated.

The results showed that IC50 for compound C in this test was >32 μg/mL.

### Example 51

Effect of Pre-Administration of a [(Ala-Lys-Pro-Ser-Tyr-Hyp-Thr-DOPA-Hyp-Lys)₂-Lys]₂-Lys (SEQ ID No: 63) on the Pharmacokinetics of Mesalazine

A solution of [(Ala-Lys-Pro-Ser-Tyr-Hyp-Thr-DOPA-Hyp-Lys)₂-Lys]₂-Lys (SEQ ID No: 63; referred to hereinafter as ‘sMAP’#) was prepared by weighing 22.5 mg and adding normal saline to 15 mL to obtain a concentration of 1.5 mg/mL. The rats were treated by administering test samples. Mesalazine suppositories were melted in a water bath at 40° C. to make the mesalazine suppositories suspension.

12 SD rats (6 males and 6 females) were supplied from Beijing Vital River Laboratory Animal Technology Co., Ltd. and housed in a barrier facility for 7 days. The housing temperature was 20-26° C., with 40%-70%, alternating 12-hours of light and darkness, and free access to food and water. The rats were randomized into 2 groups as described in Table 16 below, with 6 rats in each group (3 males and 3 females in each group).

TABLE 16

| | | Dose | Dose Volume | |
|---|---|---|---|---|
| Group | Treatment | Level | (/rat) | Dose route |
| 1 | Mesalazine | 1 g | 200 μL | rectal |
| | | | | administration |
| 2 | Mesalazine + sMAP | 1 g | 200 μL | rectal |
| | solution | | | administration |

The rats were anesthetized by 2% isoflurane inhalation. The rats were placed in the supine position, the rectum end stool was extruded and 75% alcohol cotton ball was used to disinfect the anal periphery skin area.

The first group was given Mesalazine suppositories suspensions directly, while the second group was given Mesalazine suppositories suspensions after treatment with sMAP solution enema for 15 min (the details of the enema dose and administration method are shown in Table 17 below).

TABLE 17

| | | | Dose volume | |
|---|---|---|---|---|
| | | Dose level | of sMAP | Dose |
| Group | Treatment | (/rat) | (/rat) | route |
| 1 | Mesalazine | — | — | — |
| 2 | Mesalazine + sMAP | 1.5 mg | 1000 μL | enema |

A 2 mL syringe was used to draw back the air bag cavity to ensure that there was no air in the air bag after water filling. The catheter (2-WAY 8Ch/Fr3-5 mm) was inserted into the rats anus at a depth of about 3 cm (2 cm length from the end of the balloon; the tip of the catheter was cut below the position of the urethral catheter tip, and shortened as much as possible while maintaining the integrity of the air cavity. The guide wire was removed, the urethral catheter was sealed with neutral glass glue while ensuring smooth sealing end to reduce the damage to the rectal mucosa). 1 mL syringe were used to draw up the mesalazine suppositories suspension to the disposable gavage needle (soft needles), and inserted (about 3.5 cm) into the anus, 200 μL of the content of the syringe was slowly pushed into each rat.

While the perianal skin was held to fix the catheter and the gavage needle, 1 mL of water was injected with a 2 mL syringe into the airbag cavity in order to expand it, and the needle was then quickly released to observe whether there was gel leakage.

After injection, adhesive tape was wrapped around the tail root to fix the catheter (the catheter was litigated about 3-5 cm from the anus, then cut off after ligation, and an adhesive tape was wrapped around the tail root for fixation. The fixation of the pressure-sensitive adhesive tape is based on the standard of non-looseness of the urinary catheter, which should not be too tight and cause discomfort for animals to bite. The catheter should be fixed by tape twice: the catheter should be knotted the first time, while the remaining catheter should be fixed at the end of the tail the second time. The 3 cm scale on the catheter should be at the anus, and the catheter should not be taken out when the knot is tied). The rats were put back into the cage after administration.

An anal plug was maintained in the rectum for 4 h to extend the retention time of the drug in the animal body. The anal plug dropped within 4 h after the drug was administered, and the approximate time period of the drop was recorded.

Blood samples were processed by coating all centrifuge tubes with EDTA-K2 and storing them either in a refrigerator (2-8° C.) or a cooler filled with ice, protected from light prior to use; the blood that was collected was transferred into the centrifuge tubes and stored in ice box protected from light after having been mixed manually by reversing the tubes at least 5 times. The samples were subsequently centrifuged at 1800 g for 10 minutes at 2 to 8° C. within 2 hours from blood collection. After the centrifugation, the collected plasma samples were transferred into newly labeled centrifuge tubes, aliquoted into two sets, and stored below −70° C. away from light.

Plasma concentrations of Mesalazine were analyzed using an LC-MS/MS method. AUC, Cmax, Tmax, and any other parameters as needed, were calculated using WinNonlin software. Microsoft Office Excel was used for data statistical analysis, including mean, standard deviation (SD), and coefficient of variation (CV), etc. The difference in pharmacokinetic parameters between the two groups was compared.

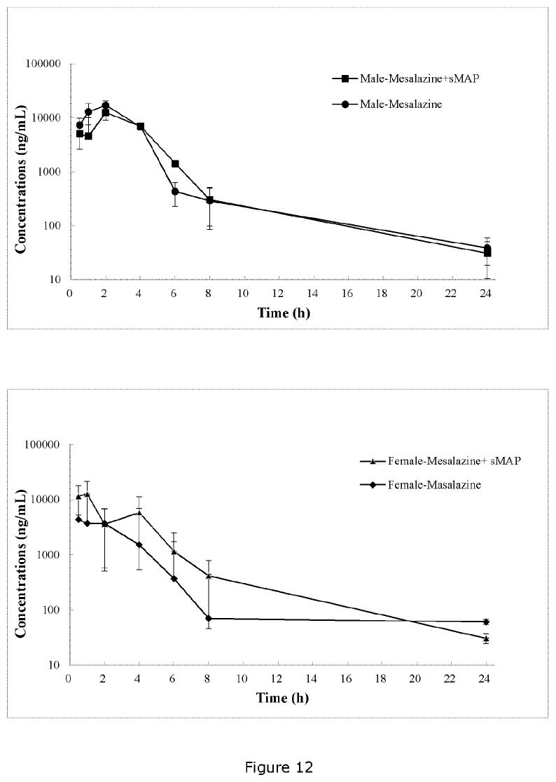

The detailed pharmacokinetics parameters are shown in Table 18 below. The plasma concentration vs time curve is illustrated in FIG. 12.

TABLE 18

| | | |
|---|---|---|
| Group 1 | Group 2 | Ratio |

Male-PK parameters

| | | | |
|---|---|---|---|
| HL_Lambda_z (T_{1/2}, h) | 6.92 | 3.17 | 2.18 |
| Tₘₐₓ (h) | 1.67 | 1.50 | 1.11 |
| Cₘₐₓ (ng/mL) | 17544.9 | 14023.2 | 1.25 |
| AUCₗₐₛₜ (h*ng/mL) | 56145.4 | 42851.8 | 1.31 |

Female-PK parameters

| | | | |
|---|---|---|---|
| HL_Lambda_z (T_{1/2}, h) | 2.74 | 1.49 | 1.84 |
| Tₘₐₓ (h) | 1.83 | 1.00 | 1.83 |
| Cₘₐₓ (ng/mL) | 13753.7 | 4553.2 | 3.02 |
| AUCₗₐₛₜ (h*ng/mL) | 38857.8 | 15236.6 | 2.55 |

The results show that the Cmax and AUCₗₐₛₜ of Mesalazine in Group 2 were lower than that of Group 1. The results indicated that when combinative administration of meselazine and sMAP (SEQ ID No: 66) in rats, the sMAP could reduce the absorption and systemic exposure of mesalazine, improve the safety and prolong the residence time of local administration to improve local efficacy.

### Example 52

### Effect of Pre-Administration of a Compound of the Invention on the Pharmacokinetics of Montelukast

The experiment was conducted in a similar fashion to that of Example 51, with the exception of using montelukast sodium suspension instead of mesalazine suppositories suspension.

The montelukast sodium suspension was prepared by weighing an appropriate amount of montelukast sodium and added into water in order to obtain a concentration of 1 mg/mL. The combination formulation was prepared by weighing an appropriate amount of montelukast sodium and synthetic MAP, and added into the water in order to obtain a suspension containing 1 mg/mL montelukast sodium and 1.5 mg/mL of sMAP, which was used right after it was ready.

12 SD rats (6 males and 6 females) were supplied from Beijing Vital River Laboratory Animal Technology Co., Ltd. and housed in a barrier facility for 7 days. The housing temperature was 20-26° C., with 40%-70%, alternating 12-hours of light and darkness, and free access to food and water. The rats were randomized into 2 groups as described in Table 19 below, with 6 rats in each group (3 males and 3 females in each group).

TABLE 19

| | | | Dose | |
|---|---|---|---|---|
| | | Dose | Volume | |
| Group | Treatment | Level | (μl/animal) | Dose route |
| 1 | Montelukast | 0.2 mg | 200 | rectal |
| | | | | administration |
| 2 | Montelukast + sMAP | 0.2 mg | 200 | rectal |
| | | | | administration |

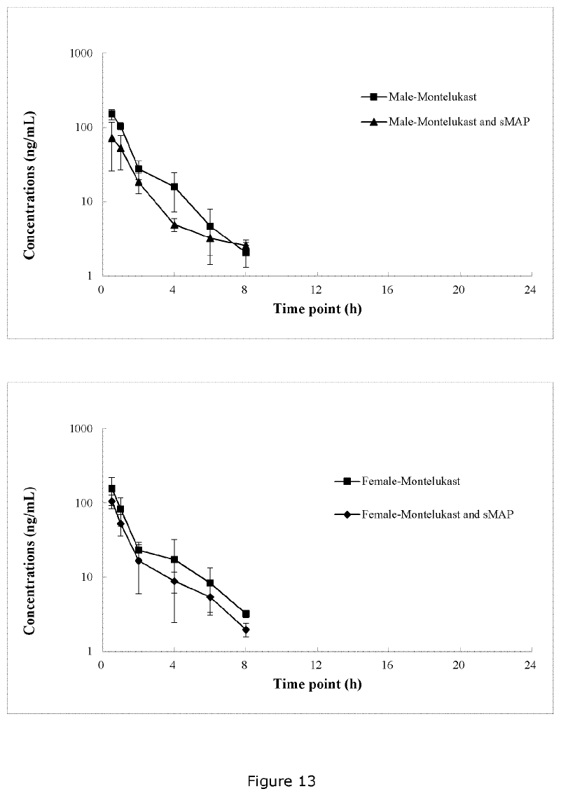

The administration of the doses, as well as the blood sample collection and pharmacokinetics analysis were conducted in the same manner as in Example 51. The detailed pharmacokinetics parameters are shown in Table 20 below, while the plasma concentration vs time curve is illustrated in FIG. 13.

TABLE 20

| | | |
|---|---|---|
| Group 1 | Group 2 | Ratio |

Male-PK parameters

| | | | |
|---|---|---|---|
| HL_Lambda_z (T_{1/2}, h) | 1.8 | 1.92 | 2.18 |
| Tₘₐₓ (h) | 0.50 | 0.50 | 1.11 |
| Cₘₐₓ (ng/mL) | 150.9 | 72.2 | 1.25 |
| AUCₗₐₛₜ (h*ng/mL) | 238.2 | 120.4 | 1.31 |

Female-PK parameters

| | | | |
|---|---|---|---|
| HL_Lambda_z (T_{1/2}, h) | 2.3 | 2.0 | 1.12 |
| Tₘₐₓ (h) | 0.50 | 0.50 | 1.00 |
| Cₘₐₓ (ng/mL) | 155.9 | 105.8 | 1.47 |
| AUCₗₐₛₜ (h*ng/mL) | 229.2 | 147.9 | 1.55 |

The results show that the Cₘₐₓ and AUCₗₐₛₜ of montelukast in Group 2 were lower than that of Group 1. The results indicated that when montelukast is administered in combination with sMAP (SEQ ID No: 66) in rats, the sMAP could reduce the absorption and systemic exposure of montelukast, improve the safety and prolong the residence time of local administration to improve local efficacy.

### Example 53

### Pre-Administration of a Compound of the Invention to Affect Stability and/or Pharmacokinetics of Different Drugs

A solution of sMAP was prepared by weighing 22.5 mg and adding normal saline to 15 mL to obtain a concentration of 1.5 mg/mL. The rats were treated by administering test samples. Test sample 1 was 5-aminosalicylic acid (mesalazine) suppositories, which were melted in a water bath at 40° C. Test sample 2 was montelukast sodium gel, which was prepared as described in Example 52.

18 SD rats (9 males and 9 females) were supplied from Beijing Vital River Laboratory Animal Technology Co., Ltd. and housed in a barrier facility for 7 days. The housing temperature was 20-26° C., with 40%-70% humidity, alternating 12-hours of light and darkness, and free access to food and water. The rats were randomly divided into 3 groups as described in Table 21, with 6 rats in each group (3 males and 3 females in each group).

TABLE 21

| | | Dose | | Dose | |
|---|---|---|---|---|---|
| Group | Treatment | Level | | Volume | Dose route |
| 1 | Mesalazine | 1 | g | 200 μl | rectal |
| | | | | | administration |
| 2 | Mesalazine + sMAP | 1 | g | 200 μl | rectal |
| | | | | | administration |
| 3 | Montelukast + sMAP | 1 | mg | 200 μl | rectal |
| | | | | | administration |

The rats were anesthetized by 2% isoflurane inhalation. The rats were placed in the supine position, the rectum end stool was extruded and 75% alcohol cotton ball was used to disinfect the anal periphery skin area. The first group was given 5-aminosaicyclic acid (mesalazine) suppository suspensions directly, while the second and third group were given montelukast sodium gel and 5-aminosaicyclic acid (mesalazine) suppository suspensions (respectively), after the animals were treated with a MAP solution enema for 15 min (the details of the enema dose and method are in Table 17 below).

TABLE 22

| | | Dose Level | Dose Volume |
|---|---|---|---|
| Group | Treatment | (per rat) | (per rat) |
| 1 | Mesalazine | — | — |
| 2 | Mesalazine + sMAP | 1.5 mg | 1000 μl |
| 3 | Montelukast + sMAP | 1.5 mg | 1000 μl |

A 2 ml syringe was used to draw back the air bag cavity to ensure that there was no air in the air bag after water filling. The catheter (2-WAY 8Ch/Fr3-5 mm) was inserted into the rat anus at a depth of about 3 cm (2 cm length from the end of the balloon; the tip of the catheter was cut below the position of the urethral catheter tip, and shortened as much as possible while maintaining the integrity of the air cavity. the guide wire was removed, the urethral catheter was sealed with neutral glass glue while ensuring smooth sealing end to reduce the damage to the rectal mucosa). 1 mL syringes were used to draw up the montelukast sodium gel (UP-611 gel (5 mg/g)) and the 5-aminosaicyclic acid (mesalazine) suppository suspension (respectively) to the disposable gavage needle (soft needles), and inserted (about 3.5 cm) into the anus, 200 μL of the content of the syringe was slowly pushed into each rat.

While the perianal skin was held to fix the catheter and the gavage needle, 1 mL of water was injected with a 2 mL syringe into the airbag cavity in order to expand it, and the needle was then quickly released to observe whether there was gel leakage.

After injection, adhesive tape was wrapped around the tail root to fix the catheter (the catheter was litigated about 3-5 cm from the anus, then cut off after ligation, and an adhesive tape was wrapped around the tail root for fixation. The fixation of the pressure-sensitive adhesive tape is based on the standard of non-looseness of the urinary catheter, which should not be too tight and cause discomfort for animals to bite. The catheter should be fixed by tape twice: the catheter should be knotted the first time, while the remaining catheter should be fixed at the end of the tail the second time. The 3 cm scale on the catheter should be at the anus, and the catheter should not be taken out when the knot is tied). The rats were put back into the cage after administration.

An anal plug was maintained in the rectum for 4 h to extend the retention time of the drug in the animal body. The anal plug dropped within 4 h after the drug was administered, and the approximate time period of the drop was recorded.

Blood samples are processed by coating all centrifuge tubes with EDTA-K2 and storing them either in a refrigerator (2-8° C.) or a cooler filled with ice, protected from light prior to use; the blood that is collected is transferred into the centrifuge tubes and stored in an ice box protected from light after having being mixed manually by reversing the tubes at least 5 times. The samples are subsequently centrifuged at 1800 rpm for 10 minutes at 2 to 8° C. within 2 hours from blood collection. After the centrifugation, the collected plasma samples are transferred into a newly labeled centrifuge tubes, aliquoted into two sets, and stored below −70° C. away from light.

Pharmacokinetics Analysis: Plasma concentration of 5-aminosaicyclic acid (mesalazine) and montelukast is analyzed using an LC-MS/MS method and relevant standard operating procedures (SOPs). AUC, Cmax, Tmax, and any other parameters as needed, are calculated using WinNonlin software. Microsoft Office Excel is used for data statistical analysis, including mean, standard deviation (SD), and coefficient of variation (CV), etc. The difference in pharmacokinetic parameters between the control group with and without MAP enema groups is compared. The detailed analysis documents are retained in the study files.

### Example 54

### A Compound of the Invention for Use as a Coating

0.01-0.5 M buffer with different pHs (6.0-9.0) (including 0.01 M sodium phosphate (pH 6.0), 0.1 M sodium bicarbonate (pH 8.5), 0.2 M sodium carbonate (pH 9.0) and 0.5 M sodium phosphate (pH 7.0), etc.) are prepared.

The buffer is added to the container the surface of which is to be coated (e.g. cell culture surface). The volume to immerse the surfaces to be coated and the total area that the buffer had covered is calculated. 90% of the total volume needed (to coat the surface) is covered by the buffer solution, while the remaining 10% of the total volume is covered by a solution (of different concentrations, e.g. 1 μg/mL to 100 mg/mL) of either one or more compounds of the invention (such as SEQ ID No: 48, 51, 54, 60, 61, 66, 67, 72, 73, etc.) or pre-crosslinked versions (see above).

The mixture of buffer and compounds of the invention (i.e. the coating solution) in the container is mixed well and left to coat the surface for about 10 minutes to 48 hours at 4-80° C. Then, the coating solution is poured or transferred out and the surface is washed with water (using the same total volume as that of the coating solution).

The coated density (mg/cm²) could be roughly calculated by the total amount (mg) of sMAPs or pre-crosslinked sMAPs added and divided by the total area (cm²) that the solution has covered.

### Example 55

(SEQ ID No. 70)
{[(DOPA-Ala-Lys-Pro-Ser-Tyr-Hyp-Thr-Tyr-Hyp-Lys)₂-
Lys]₂-Lys}₂-Lys Coated Cell Culture Plates

Using essentially the same method as that described in Example 54 above, 0.1 M sodium bicarbonate (pH 8.5) solution is freshly prepared by dissolving 8.4 g sodium bicarbonate in pure water to a total volume of 1 L, the pH of the solution is about 8.5.

Two corning costar 24-well cell culture plates are prepared. To be able to coat the bottom surface of the cell culture plates, a total volume of 400 μL solution is enough for each well. The total surface area to be covered by the solution is about 3 cm². 360 μL of 0.1 M sodium bicarbonate (pH 8.5) solution (as prepared above) is added to each well that is to be coated.

10 mL of {[(DOPA-Ala-Lys-Pro-Ser-Tyr-Hyp-Thr-Tyr-Hyp-Lys)₂-Lys]₂-Lys}2-Lys (SEQ ID No: 70) solutions at 2.25 mg/mL and 1.125 mg/mL are prepared by dissolving 22.5 mg and 11.25 mg respectively of the peptide in pure water to a final volume of 10 mL separately. 40 μL of 2.25 mg/mL peptide solution is added to one corning costar 24-well cell culture plates pre-filled with 0.1 M sodium bicarbonate (pH 8.5) solution and mixed well. 40 μL of 1.125 mg/mL peptide solution is added to the other corning costar 24-well cell culture plates pre-filled with 0.1 M sodium bicarbonate (pH 8.5) solution and mixed well. Then, both plates are left at room temperature, letting the solution coat for 18 hours. The coating solution is poured out and the coated well is washed with the same volume of pure water as coating solution once.

The coated density (mg/cm²) of coated cell culture plates may be roughly calculated by the total amount (mg) of the peptide added, dividing by the total areas (cm²) the solution had covered. Consequently, the coated density is 30 μg/cm² and 15 μg/cm² respectively.

### Example 56

A Compound of the Invention as Carrier for Heparin Sodium for Transfusion Management

A 1 mg/mL solution of [(DOPA-Ala-Lys-Pro-Ser-Tyr-Hyp-Thr-Tyr-Hyp-Lys)₂-Lys]₂-Lys (SEQ ID No: 64) is obtained using the method described in Example 55 above in the previous example by dissolving it in purified water.

The peptide solution is pumped into the catheter at the flow rate of 0.5 mL/min, and the solution is left in the catheter for 30 minutes and dried at room temperature.

Heparin sodium powder (185 USP units/mg, Aladdin) is made into a solution of 200 μg/ml with normal saline. The heparin sodium solution is pumped into the catheter containing a peptide layer at a flow rate of 0.1 mL/min. Heparin sodium is adsorbed to the inner wall of the catheter by electrostatic interaction.

The catheters prepared by the above methods can be used in blood transfusion systems such as ECMO.

### Example 57

### Preparation of Omeprazole Enteric Coated Tablets

Each tablet contains 7.5 mg of sMAP and 20 mg of omeprazole. The formulation is based on 10,000 tablets. Table 21 shows the various steps and amounts of each ingredient in the formulation.

TABLE 23

| | Auxiliary | | |
|---|---|---|---|
| | material | Name of raw and | Dosage |
| Steps | function | auxiliary materials | (unit: g) |
| Omeprazole | Active | Omeprazole | 200 |
| granules | ingredient | | |
| | Surface active | Tween 80 | 2 |
| | agent | | |
| | Alkaline | Sodium dihydrogen | 30 |
| | stabilizer | phosphate | |
| | Filler | Microcrystalline cellulose | 1010 |
| | | mannitol | 200 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1500 |
| Oxazole | Core | Omeprazole granules | 1500 |
| coated | Coating | Hydroxypropyl | 100 |
| granules | material | methylcellulose | |
| | solvent | water | 4500 |
| | Total weight | | 1600 |
| sMAP granules | Active | sMAP | 75 |
| | Ingredient | | |
| | Filler | Microcrystalline cellulose | 1100 |
| | | mannitol | 367 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1600 |
| Plain tablets | Medicinal | Oxazole coated granules | 1600 |
| | granules | | |
| | Medicinal | sMAP granules | 1600 |
| | granules | | |
| | lubricant | Magnesium stearate | 32 |
| | Total weight | | 3232 |
| Separation | Core | Plain tablets | 3232 |
| layer on tablet | Coating | Hydroxypropyl | 100 |
| surface | material | methylcellulose | |
| | solvent | water | 3332 |
| Enteric layer | Core | Omeprazole tablets with | 3332 |
| | | spacer | |
| | Enteric coated | EUDRAGIT L30D-55 | 55 |
| | materials | | |
| | Plasticizer | Polyethylene glycol 400 | 5 |
| | Solvent | water | 1700 |

### Omeprazole Granules

Disodium hydrogen phosphate and Tween 80 are dissolved in purified water by stirring. Omeprazole is added and the mixture is mixed evenly. After adding microcrystalline cellulose, mannitol, hydroxypropyl methylcellulose and low substituted hydroxypropyl cellulose into the mixture, the prepared omeprazole solution is added and stirred evenly, granulated, dried and set aside.

### Omeprazole Particle Coated Spacer

Hydroxypropyl methylcellulose is added into purified water slowly and stirred until it is clear and transparent. The omeprazole granules are put into the coating machine for coating. After coating, the materials are dried at 40-50° C.

sMAP Granules

sMAP is dissolved in purified water, and then stirred to dissolve it. After mixing the formulation amount of microcrystalline cellulose, mannitol, hydroxypropyl methylcellulose and low substituted hydroxypropyl cellulose, the prepared sMAP solution is added, granulated, dried and pelleted.

### The Tablet

After mixing omeprazole coated granules with sMAP granules, magnesium stearate is added and the suspension is mixed and, subsequently, compressed.

### Isolation Coating

Hydroxypropyl methylcellulose is added into purified water slowly and the mixture is stirred until it is clear and transparent. The omeprazole tablets are put into the coating machine to coat and then they are dried.

### Enteric Coating

Polyethylene glycol is dissolved in purified water. Eudragit 130d-55 ss added, and the mixture is stirred evenly, and sieved. The core is put into the coating pot, coated, dried, and sampled for inspection.

### Example 58

### Preparation of Famotidine Enteric Coated Tablets

Each tablet contains 7.5 mg of sMAP and 20 mg of famotidine. The formulation is based on 10,000 tablets. Table 22 shows the various steps and amounts of each ingredient in the formulation.

TABLE 24

| | Auxiliary | | |
|---|---|---|---|
| | material | Name of raw and | Dosage |
| Step | function | auxiliary materials | (unit: G) |
| Famotidine | Active | Famotidine | 200 |
| granules | ingredient | | |
| | Surface active | Tween 80 | 2 |
| | agent | | |
| | Filler | Microcrystalline cellulose | 1040 |
| | | mannitol | 200 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1500 |
| sMAP granules | Active | sMAP | 75 |
| | ingredient | | |
| | Filler | Microcrystalline cellulose | 367 |
| | | lactose | 550 |
| | | mannitol | 550 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1600 |
| Plain tablets | Medicinal | Famotidine Granules | 1600 |
| | granules | | |
| | Medicinal | sMAP granules | 1600 |
| | granules | | |
| | Lubricant | Magnesium stearate | 32 |
| | Total weight | | 3232 |
| Separation | Core | Plain tablets | 3232 |
| layer on tablet | Coating | Hydroxypropyl | 100 |
| surface | material | methylcellulose | |
| | solvent | water | 3332 |
| Enteric layer | Core | Plain sheet with spacer | 3332 |
| | Enteric coated | EUDRAGIT L30D-55 | 55 |
| | materials | | |
| | Plasticizer | Polyethylene glycol 400 | 5 |
| | Solvent | water | 1700 |

### Famotidine Granules

Tween 80 is added to purified water, stirred and dissolved. Famotdine is added into the mixture evenly. After adding microcrystalline cellulose, mannitol, hydroxypropyl methylcellulose and low substituted hydroxypropyl cellulose into the mixture, the prepared omeprazole solution is added, the mixture is stirred evenly, granulated, dried and set aside.

sMAP Granules

sMAP is dissolved in purified water and then stirred to dissolve. After mixing microcrystalline cellulose, mannitol, lactose, hydroxypropyl methylcellulose and low substituted hydroxypropyl cellulose, the prepared sMAP solution is added, granulated, dried and pelleted.

### The Tablet

Famotidine granules are mixed with sMAP granules. Magnesium stearate is added and the mixture is mixed and, subsequently, compressed.

### Isolation Coating

Hydroxypropyl methylcellulose is added into purified water slowly and stirred until it is clear and transparent. The famotidine tablets are put into the coating machine, coated and dried.

### Enteric Coating

Polyethylene glycol is dissolved in purified water, Eudragit 130d-55 is added and the mixture is stirred evenly, and sieved. The core is put into the coating pot, coated, dried, and sampled for inspection.

### Example 59

### Preparation of Omeprazole Enteric Coated Capsules

Each capsule contains 7.5 mg of sMAP and 20 mg of omeprazole. The formulation is calculated based on 10,000 pills. Table 23 shows the various steps and amounts of each ingredient in the formulation.

TABLE 23

| | Auxiliary | | |
|---|---|---|---|
| | material | Name of raw and | Dosage |
| Steps | function | auxiliary materials | (unit: G) |
| Omeprazole | Active | omeprazole | 200 |
| granules | ingredient | | |
| | Surface active | Tween 80 | 2 |
| | agent | | |
| | Alkaline | Sodium dihydrogen | 30 |
| | stabilizer | phosphate | |
| | Filler | Microcrystalline cellulose | 710 |
| | | dextrin | 400 |
| | | mannitol | 200 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1500 |
| Oxazole | Core | Omeprazole granules | 1500 |
| coated | Coating | Hydroxypropyl | 100 |
| granules | material | methylcellulose | |
| | Solvent | water | 4500 |
| | Total weight | | 1600 |
| sMAP | Active \| | sMAP | 75 |
| granules | ingredient | | |
| | Filler | Microcrystalline cellulose | 367 |
| | | mannitol | 1100 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1600 |
| capsule | Medicinal | Oxazole coated granules | 1600 |
| | granules | | |
| | Medicinal | sMAP granules | 1600 |
| | granules | | |
| | Lubricant | Magnesium stearate | 32 |
| | Capsule shell | Enteric coated gelatin | 10000(grain) |
| | | hollow capsules | |

| | |
|---|---|
| Total weight | 3232 |

### Omeprazole Granules

Disodium hydrogen phosphate and Tween 80 are added to purified water, stirred and dissolved. Omeprazole is added and the mixture is stirred and dispersed evenly. Microcrystalline cellulose, dextrin, mannitol, hydroxypropyl methylcellulose and low substituted hydroxypropyl cellulose are added into the mixture, and then added into the solution for granulation, drying and pelleting. It is set aside.

### Omeprazole Package Spacer

Hydroxypropyl methylcellulose is added to purified water slowly and stirred until it is clear and transparent. The omeprazole granules are put into the coating machine for coating. After coating, the materials are dried at 40-50° C.

sMAP Granules

sMAP is dissolved in purified water, and then stirred to dissolve. After mixing microcrystalline cellulose, mannitol, hydroxypropyl methylcellulose and low substituted hydroxypropyl cellulose, the solution is added, whereupon granulation, drying and pelleting are carried out.

### Filling Capsule

Omeprazole coated particles and sMAP granules are mixed. Magnesium stearate is added, and then the enteric coated capsules are filled with the mixture.

### Example 60

### Preparation of Famotidine Enteric Coated Capsules

Each capsule contains 7.5 mg of sMAP and 20 mg of famotidine. The formulation is based on 10,000 pills. Table 24 below shows the various steps and amounts added of each ingredient into the formulation.

TABLE 24

| | Auxiliary | | |
|---|---|---|---|
| | material | Name of raw and | Dosage |
| step | function | auxiliary materials | (unit: G) |
| Famotidine | Active | Famotidine | 200 |
| Granules | ingredient | | |
| | Surface active | Tween 80 | 2 |
| | agent | | |
| | Filler | Microcrystalline cellulose | 1040 |
| | | mannitol | 200 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1500 |
| sMAP | Active | sMAP | 75 |
| granules | ingredient | | |
| | Filler | Microcrystalline cellulose | 367 |
| | | mannitol | 1100 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1600 |
| capsule | Medicinal | Famotidine Granules | 1600 |
| | granules | | |
| | Medicinal | sMAP granules | 1600 |
| | granules | | |
| | Lubricant | Magnesium stearate | 32 |
| | Shell capsule | Enteric coated gelatin | 10000(grain) |
| | | hollow capsules | |

| | |
|---|---|
| Total weight | 3232 |

### Famotidine Granules

Tween 80 is added to purified water, and the solution is stirred to dissolve. Modine is added into the mixture evenly. Microcrystalline cellulose, mannitol, hydroxypropyl methylcellulose and low substituted hydroxypropyl cellulose are mixed, then added into the solution, whereupon granulation, drying and pelleting is conducted. It is set aside.

### Famotidine Granules Coated Isolation Layer

Hydroxypropyl methylcellulose is added into purified water slowly and stirred until the mixture is clear and transparent. The famotidine granules are put into the coating machine for coating. After coating, the materials are dried at 40-50° C.

sMAP Granules

sMAP is dissolved in purified water, and the mixture is stirred to dissolve. Microcrystalline cellulose, mannitol, hydroxypropyl methylcellulose and low substituted hydroxypropyl cellulose, are mixed and then added to the solution, whereupon granulation, drying and pelleting are carried out.

### Filling Capsule

Famotidine granules and sMAP granules are mixed. Magnesium stearate is added, and the mixture is then used to fill the enteric coated capsules.

### Example 61

### Preparation of Omeprazole/sMAP Capsule for Use in Proctitis

Table 25 below shows the various steps and amounts added of each ingredient into the formulation.

TABLE 25

| | Auxiliary | | |
|---|---|---|---|
| | material | Name of raw and | Dosage |
| Steps | function | auxiliary materials | (unit: G) |
| Omeprazole | Active | omeprazole | 200 |
| granules | ingredient | | |
| | Surface active | Tween 80 | 2 |
| | agent | | |
| | Alkaline | Sodium dihydrogen | 30 |
| | stabilizer | phosphate | |
| | Filler | Microcrystalline cellulose | 1010 |
| | | mannitol | 200 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1500 |
| Oxazole | Core | Omeprazole granules | 1500 |
| coated | Coating | Hydroxypropyl | 100 |
| granules | material | methylcellulose | |
| | Solvent | water | 4500 |
| | Total weight | | 1600 |
| sMAP | Active | sMAP | 75 |
| granules | ingredient | | |
| | Filler | Microcrystalline cellulose | 367 |
| | | mannitol | 1100 |
| | Adhesive | Hydroxypropyl | 50 |
| | | methylcellulose | |
| | Disintegrating | Low substituted | 8 |
| | agent | hydroxypropyl | |
| | | methylcellulose | |
| | Solvent | Purified water | 400 |
| | Total weight | | 1600 |
| Capsule | Medicinal | Oxazole coated granules | 1600 |
| | granules | | |
| | Medicinal | sMAP granules | 1600 |
| | granules | | |
| | Lubricant | Magnesium stearate | 32 |
| | Capsule shell | Hydroxypropyl | 10000(grain) |
| | | methylcellulose hollow | |
| | | capsules | |

| | |
|---|---|
| Total weight | 3232 |

### Omeprazole Granules

Disodium hydrogen phosphate and Tween 80 were added to purified water. The mixture was stirred and dissolved. Omeprazole was stirred into the mixture and dispersed evenly. Microcrystalline cellulose, mannitol, hydroxypropyl methylcellulose and low substituted hydroxypropyl cellulose were mixed, then added into the solution, whereupon granulation, drying and pelleting were conducted. It was set aside.

### Omeprazole Particle Coated Spacer

Hydroxypropyl methylcellulose was added to purified water slowly and the mixture was stirred until it was clear and transparent. The omeprazole granules were put into the coating machine for coating. After coating, the materials were dried at 40-50° C.

sMAP Granules

sMAP was dissolved in purified water, and then stirred to dissolve. Microcrystalline cellulose, mannitol, hydroxypropyl methylcellulose and low substituted hydroxypropyl cellulose, were mixed and then added to the solution, whereupon granulation, drying and pelleting were carried out.

### Filling Capsule

After mixing omeprazole coated granules and sMAP granules, magnesium stearate was added to the mixture, and then hydroxypropyl methylcellulose hollow capsules were filled with the mixture.

1. A compound of formula I,
A-Q-B I
wherein:
A and B independently represent Z or A¹-Q¹-B¹;
Q represents a structural fragment of formula II, 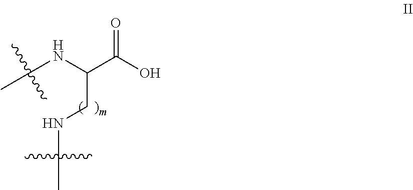
wherein:
the squiggly lines represent points of attachment of Q to A and/or B; and
m represents an integer 1 to 4;
A¹ and B¹ independently represent Z or A²-Q²-B²;
A² and B² independently represent Z or Z-Q³-Z;
Q¹, Q² and Q³ independently represent structural fragments of formula III, 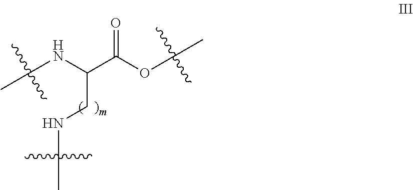
wherein:
the squiggly lines adjacent to the NH groups represent the points of attachment of Q¹, Q² and Q³ to A¹ and/or B¹, A² and/or B², and Z, respectively;
the squiggly line adjacent to the O atom represents the point of attachment of Q¹, Q² and Q³ to Q, Q¹ and Q², respectively; and
m is as defined above;
on each occasion that it is employed, Z represents a peptide component of the amino acid sequence:
(SEQ ID No: 3)
[W-Lys-X¹-Ser-U-X²-Y]ₙ-W-Lys-X¹-Ser-U-X²-Y
wherein:
the dashed line represents the point of attachment of Z to the rest of the molecule;
n represents 0 or an integer 1 to 4; and,
on each occasion that they are employed:
W represents a 1 or 2 amino acid sequence, in which the amino acids are selected from one or more of the group Lys, Ala, DOPA and a 3,4-dihydrocinnamic acid (HCA) residue, provided that, when present, the HCA residue is located at the N-terminus of the peptide sequence Z;
X¹ represents Pro, Hyp or diHyp;
U represents Tyr or DOPA;
X² represents Ser, Pro, Hyp or diHyp; and
Y represents a 1 to 5 amino acid sequence, in which the amino acids are selected from one or more of the group Lys, Ala, Pro, Hyp, diHyp, Thr, DOPA and Tyr, as well as regioisomers, stereoisomers, and pharmaceutically- or cosmetically-acceptable salts of said compound.

2. The compound as claimed in claim 1, wherein m represents 4.

3. The compound as claimed in claim 1, wherein A and B both represent Z, or both represent A¹-Q¹-B¹.

4. The compound as claimed in claim 1, wherein A¹ and B¹ both represent Z, or both represent A²-Q²-B².

5. The compound as claimed in claim 1, wherein A² and B² both represent Z, or both represent Z-Q³-Z.

6. The compound as claimed in claim 1, wherein n is 0.

7. The compound as claimed in claim 1, wherein X¹ represents Pro.

8. The compound as claimed in claim 1, wherein X² represents Hyp.

9. The compound as claimed in claim 1, wherein W is selected from the group HCA, HCA-Ala-, Ala, DOPA, Lys-Ala and DOPA-Ala.

10. The compound as claimed in claim 1, wherein Y represents a 4 amino acid sequence, in which the amino acids are selected from one or more of the group Lys, Ala, Hyp, Thr, DOPA and Tyr.

11. The compound as claimed in claim 10, wherein Y represents an amino acid sequence selected from the group -Hyp-Y¹-Y²-Lys- (SEQ ID NO: 87) and -Thr-Y¹-Y²-Lys- (SEQ ID NO: 88), wherein Y¹ and Y² are each independently selected from the group Ala, Hyp, Thr, DOPA and Tyr.

12. The compound as claimed in claim 10, wherein the amino acid sequence defined by Y is selected from the group -Hyp-Thr-Tyr-Lys- (SEQ ID NO: 93), -Hyp-Thr-DOPA-Lys- (SEQ ID NO: 94), -Hyp-Thr-Ala-Lys- (SEQ ID NO: 95), -Thr-Tyr-Hyp-Lys- (SEQ ID NO: 96), -Thr-DOPA-Hyp-Lys- (SEQ ID NO: 97), and -Thr-Ala-Hyp-Lys- (SEQ ID NO: 98).

13. The compound as claimed in claim 1, wherein the amino acid sequence defined by Y is selected from the group -Thr-Tyr-Hyp-Lys-DOPA- (SEQ ID NO: 100) and -Hyp-Thr-DOPA-.

14. The compound as claimed in claim 1, wherein U represents Tyr.

15. (canceled)

16. The compound as claimed in claim 1, wherein Z is selected from the group:
(SEQ ID No: 2)
Ala-Lys-Pro-Ser-Tyr-Hyp-Hyp-Thr-Tyr-Lys;
(SEQ ID No: 1)
Ala-Lys-Pro-Ser-Tyr-Hyp-Hyp-Thr-DOPA-Lys;
(SEQ ID No: 4)
Ala-Lys-Pro-Ser-Tyr-Hyp-Thr-Tyr-Hyp-Lys;
(SEQ ID No: 5)
Ala-Lys-Pro-Ser-Tyr-Hyp-Thr-DOPA-Hyp-Lys;
(SEQ ID No: 6)
Ala-Lys-Pro-Ser-Tyr-Hyp-Thr-Tyr-Hyp-Lys-DOPA;
(SEQ ID No: 7)
Ala-Lys-Pro-Ser-Tyr-Ser-Hyp-Thr-Tyr-Lys-Ala-Lys-
Pro-Ser-Tyr-Ser-Hyp-Thr-Tyr-Lys-;
(SEQ ID No: 8)
Ala-Lys-Pro-Ser-Tyr-Hyp-Hyp-Thr-Tyr-Lys-Ala-Lys-
Pro-Ser-Tyr-Hyp-Hyp-Thr-Tyr-Lys-Ala-Lys-Pro-Ser-
Tyr-Hyp-Hyp-Thr-Tyr-Lys-Ala-Lys-Pro-Ser-Tyr-Hyp-
Hyp-Thr-Tyr-Lys-Ala-Lys-Pro-Ser-Tyr-Hyp-Hyp-Thr-
Tyr-Lys;
(SEQ ID No: 9)
Lys-Ala-Lys-Pro-Ser-Tyr-Hyp-Hyp-Thr-Tyr;
(SEQ ID No: 10)
Lys-Ala-Lys-Hyp-Ser-Tyr-Hyp-Hyp-Thr-DOPA;
and
(SEQ ID No: 11)
Lys-Ala-Lys-Pro-Ser-Tyr-Hyp-Hyp-Thr-DOPA.

17. (canceled)

18. The compound as claimed in claim 1, wherein W represents HCA, HCA-Ala-, DOPA or DOPA-Ala-, and Z is selected from the group:
(SEQ ID No: 12)
HCA-Ala-Lys-Pro-Ser-Tyr-Hyp-Thr-Tyr-Hyp-Lys;
(SEQ ID No: 13)
HCA-Ala-Lys-Pro-Ser-Tyr-Hyp-Hyp-Thr-Tyr-Lys;
(SEQ ID No: 14)
HCA-Lys-Pro-Ser-Tyr-Hyp-Hyp-Thr-Ala-Lys;
(SEQ ID No: 15)
HCA-Lys-Pro-Ser-Tyr-Hyp-Thr-Ala-Hyp-Lys;
(SEQ ID No: 16)
DOPA-Lys-Pro-Ser-Tyr-Hyp-Thr-Ala-Hyp-Lys;
(SEQ ID No: 17)
DOPA-Lys-Pro-Ser-Tyr-Hyp-Hyp-Thr-Ala-Lys;
(SEQ ID No: 18)
DOPA-Ala-Lys-Pro-Ser-Tyr-Hyp-Thr-Tyr-Hyp-Lys;
and
(SEQ ID No: 19)
DOPA-Ala-Lys-Pro-Ser-Tyr-Hyp-Hyp-Thr-Tyr-Lys.

19. (canceled)

20. The compound as claimed in claim 1, wherein U represents DOPA and Z is selected from the group:
(SEQ ID No: 20)
Ala-Lys-Pro-Ser-DOPA-Hyp-Thr-DOPA-Hyp-Lys;
(SEQ ID No: 21)
Ala-Lys-Pro-Ser-DOPA-Hyp-Hyp-Thr-DOPA-Lys;
(SEQ ID No: 22)
Lys-Ala-Lys-Pro-Ser-DOPA-Hyp-Hyp-Thr-DOPA;
(SEQ ID No: 23)
Lys-Ala-Lys-Hyp-Ser-DOPA-Hyp-Hyp-Thr-DOPA;
(SEQ ID No: 24)
Ala-Lys-Pro-Ser-DOPA-Hyp-Hyp-Thr-DOPA-Lys-Ala-
lys-Pro-Ser-DOPA-Hyp-Hyp-Thr-DOPA-Lys;
and
(SEQ ID No: 25)
Ala-Lys-Pro-Ser-DOPA-Hyp-Hyp-Thr-DOPA-Lys-Ala-
Lys-Pro-Ser-DOPA-Hyp-Hyp-Thr-DOPA-Lys-Ala-Lys-
Pro-Ser-DOPA-Hyp-Hyp-Thr-DOPA-Lys-Ala-Lys-Pro-
Ser-DOPA-Hyp-Hyp-Thr-DOPA-Lys-Ala-Lys-Pro-Ser-
DOPA-Hyp-Hyp-Thr-DOPA-Lys.

21. The compound as claimed in claim 1, wherein Z is selected from the group:
(SEQ ID No: 26)
HCA-Ala-Lys-Pro-Ser-DOPA-Hyp-Thr-DOPA-Hyp-Lys;
(SEQ ID No: 27)
HCA-Ala-Lys-Pro-Ser-DOPA-Hyp-Hyp-Thr-DOPA-Lys;
(SEQ ID No: 28)
HCA-Lys-Pro-Ser-DOPA-Hyp-Thr-Ala-Hyp-Lys;
(SEQ ID No: 29)
HCA-Lys-Pro-Ser-DOPA-Hyp-Hyp-Thr-Ala-Lys;
(SEQ ID No: 30)
DOPA-Lys-Pro-Ser-DOPA-Hyp-Thr-Ala-Hyp-Lys;
(SEQ ID No: 31)
DOPA-Lys-Pro-Ser-DOPA-Hyp-Hyp-Thr-Ala-Lys;
(SEQ ID No: 32)
DOPA-Ala-Lys-Pro-Ser-DOPA-Hyp-Thr-DOPA-Hyp-Lys;
(SEQ ID No: 33)
DOPA-Ala-Lys-Pro-Ser-DOPA-Hyp-Hyp-Thr-Tyr-Lys;
and
(SEQ ID No: 34)
DOPA-Ala-Lys-Pro-Ser-DOPA-Hyp-Hyp-Thr-DOPA-Lys.

22. The compound as claimed in claim 1, wherein A and B both represent Z, one or both Z groups represent:
(SEQ ID No: 4)
Ala-Lys-Pro-Ser-Tyr-Hyp-Thr-Tyr-Hyp-Lys,
(SEQ ID No: 5)
Ala-Lys-Pro-Ser-Tyr-Hyp-Thr-DOPA-Hyp-Lys,
(SEQ ID No: 12)
HCA-Ala-Lys-Pro-Ser-Tyr-Hyp-Thr-Tyr-Hyp-Lys,
(SEQ ID No: 15)
HCA-Lys-Pro-Ser-Tyr-Hyp-Thr-Ala-Hyp-Lys,
(SEQ ID No: 18)
DOPA-Ala-Lys-Pro-Ser-Tyr-Hyp-Thr-Tyr-Hyp-Lys,
(SEQ ID No: 20)
Ala-Lys-Pro-Ser-DOPA-Hyp-Thr-DOPA-Hyp-Lys,
(SEQ ID No: 22)
Lys-Ala-Lys-Pro-Ser-DOPA-Hyp-Hyp-Thr-DOPA
(SEQ ID No: 26)
HCA-Ala-Lys-Pro-Ser-DOPA-Hyp-Thr-DOPA-Hyp-Lys,
(SEQ ID No: 31)
DOPA-Lys-Pro-Ser-DOPA-Hyp-Hyp-Thr-Ala-Lys,
(SEQ ID No: 32)
DOPA-Ala-Lys-Pro-Ser-DOPA-Hyp-Thr-DOPA-Hyp-Lys,
(SEQ ID No: 2)
Ala-Lys-Pro-Ser-Tyr-Hyp-Hyp-Thr-Tyr-Lys,
or
(SEQ ID No: 1)
Ala-Lys-Pro-Ser-Tyr-Hyp-Hyp-Thr-DOPA-Lys;
and
Q represents a Lys residue.

23. The compound as claimed in claim 1, wherein A and B both represent A¹-Q¹-B¹, A¹ and B¹ both represent Z, and one or both Z groups represent:
(SEQ ID No: 4)
Ala-Lys-Pro-Ser-Tyr-Hyp-Thr-Tyr-Hyp-Lys,
(SEQ ID No: 5)
Ala-Lys-Pro-Ser-Tyr-Hyp-Thr-DOPA-Hyp-Lys,
(SEQ ID No: 11)
Lys-Ala-Lys-Pro-Ser-Tyr-Hyp-Hyp-Thr-DOPA,
(SEQ ID No: 12)
HCA-Ala-Lys-Pro-Ser-Tyr-Hyp-Thr-Tyr-Hyp-Lys,
(SEQ ID No: 13)
HCA-Ala-Lys-Pro-Ser-Tyr-Hyp-Hyp-Thr-Tyr-Lys,
(SEQ ID No: 18)
DOPA-Ala-Lys-Pro-Ser-Tyr-Hyp-Thr-Tyr-Hyp-Lys,
(SEQ ID No: 20)
Ala-Lys-Pro-Ser-DOPA-Hyp-Thr-DOPA-Hyp-Lys,
(SEQ ID No: 21)
Ala-Lys-Pro-Ser-DOPA-Hyp-Hyp-Thr-DOPA-Lys,
(SEQ ID No: 10)
Lys-Ala-Lys-Hyp-Ser-Tyr-Hyp-Hyp-Thr-DOPA
(SEQ ID No: 28)
HCA-Lys-Pro-Ser-DOPA-Hyp-Thr-Ala-Hyp-Lys,
(SEQ ID No: 30)
DOPA-Lys-Pro-Ser-DOPA-Hyp-Thr-Ala-Hyp-Lys,
(SEQ ID No: 32)
DOPA-Ala-Lys-Pro-Ser-DOPA-Hyp-Thr-DOPA-Hyp-Lys,
(SEQ ID No: 2)
Ala-Lys-Pro-Ser-Tyr-Hyp-Hyp-Thr-Tyr-Lys,
or
(SEQ ID No: 1)
Ala-Lys-Pro-Ser-Tyr-Hyp-Hyp-Thr-DOPA-Lys;
and
Q¹ represents a Lys residue.

24. The compound as claimed in claim 1, wherein A and B both represent A¹-Q¹-B¹, A¹ and B¹ both represent A²-Q²-B², A² and B² both represent Z, and one or both Z groups represent:
(SEQ ID No: 4)
Ala-Lys-Pro-Ser-Tyr-Hyp-Thr-Tyr-Hyp-Lys,
(SEQ ID No: 5)
Ala-Lys-Pro-Ser-Tyr-Hyp-Thr-DOPA-Hyp-Lys,
(SEQ ID No: 12)
HCA-Ala-Lys-Pro-Ser-Tyr-Hyp-Thr-Tyr-Hyp-Lys,
(SEQ ID No: 18)
DOPA-Ala-Lys-Pro-Ser-Tyr-Hyp-Thr-Tyr-Hyp-Lys,
(SEQ ID No: 21)
Ala-Lys-Pro-Ser-DOPA-Hyp-Hyp-Thr-DOPA-Lys,
(SEQ ID No: 32)
DOPA-Ala-Lys-Pro-Ser-DOPA-Hyp-Thr-DOPA-Hyp-Lys,
(SEQ ID No: 19)
DOPA-Ala-Lys-Pro-Ser-Tyr-Hyp-Hyp-Thr-Tyr-Lys,
(SEQ ID No: 2)
Ala-Lys-Pro-Ser-Tyr-Hyp-Hyp-Thr-Tyr-Lys,
or
(SEQ ID No: 1)
Ala-Lys-Pro-Ser-Tyr-Hyp-Hyp-Thr-DOPA-Lys;
and
Q¹ and Q² both represent Lys residues.

25. (canceled)

26. A peptide compound of the amino acid sequence:
(SEQ ID NO: 35)
[Ala-Lys-X¹-Ser-U-X²-Y]ₚ-Ala-Lys-X¹-Ser-U-X¹-Y-G
wherein
p represents an integer 1 to 4;
G is absent or represents DOPA or dopamine;
X¹ represents Pro, Hyp or diHyp;
U represents Tyr or DOPA;
X² represents Ser, Pro, Hyp or diHyp; and
Y represents a 1 to 5 amino acid sequence, in which the amino acids are selected from one or more of the group Lys, Ala, Pro, Hyp, diHyp, Thr, DOPA and Tyr
as well as regioisomers, stereoisomers, and pharmaceutically- or cosmetically-acceptable salts of said compound.

27. (canceled)

28. The compound as claimed in claim 26 having the amino acid sequence:
(SEQ ID No: 36)
Ala-Lys-Pro-Ser-Tyr-Ser-Hyp-Thr-Tyr-Lys-Ala-Lys-
Pro-Ser-Tyr-Ser-Hyp-Thr-Tyr-Lys;
(SEQ ID No: 37)
Ala-Lys-Pro-Ser-Tyr-Hyp-Hyp-Thr-Tyr-Lys-Ala-Lys-
Pro-Ser-Tyr-Hyp-Hyp-Thr-Tyr-Lys-Ala-Lys-Pro-Ser-
Tyr-Hyp-Hyp-Thr-Tyr-Lys-Ala-Lys-Pro-Ser-Tyr-Hyp-
Hyp-Thr-Tyr-Lys-Ala-Lys-Pro-Ser-Tyr-Hyp-Hyp-Thr-
Tyr-Lys;
(SEQ ID No: 38)
Ala-Lys-Pro-Ser-DOPA-Hyp-Hyp-Thr-DOPA-Lys-Ala-
Lys-Pro-Ser-DOPA-Hyp-Hyp-Thr-DOPA-Lys;
(SEQ ID No: 39)
Ala-Lys-Pro-Ser-DOPA-Hyp-Hyp-Thr-DOPA-Lys-Ala-
Lys-Pro-Ser-DOPA-Hyp-Hyp-Thr-DOPA-Lys-Ala-Lys-
Pro-Ser-DOPA-Hyp-Hyp-Thr-DOPA-Lys-Ala-Lys-Pro-
Ser-DOPA-Hyp-Hyp-Thr-DOPA-Lys-Ala-Lys-Pro-Ser-
DOPA-Hyp-Hyp-Thr-DOPA-Lys;
(SEQ ID No: 40)
Ala-Lys-Pro-Ser-Tyr-Hyp-Hyp-Thr-DOPA-Lys-Ala-
Lys-Pro-Ser-Tyr-Hyp-Hyp-Thr-DOPA-Lys;
(SEQ ID No: 41)
Ala-Lys-Pro-Ser-Tyr-Hyp-Hyp-Thr-DOPA-Lys-Ala-
Lys-Pro-Ser-Tyr-Hyp-Hyp-Thr-DOPA-Lys-Ala-Lys-
Pro-Ser-Tyr-Hyp-Hyp-Thr-DOPA-Lys;
(SEQ ID No: 42)
Ala-Lys-Pro-Ser-Tyr-Hyp-Hyp-Thr-DOPA-Lys-Ala-
Lys-Pro-Ser-Tyr-Hyp-Hyp-Thr-DOPA-Lys-Ala-Lys-
Pro-Ser-Tyr-Hyp-Hyp-Thr-DOPA-Lys-Ala-Lys-Pro-
Ser-Tyr-Hyp-Hyp-Thr-DOPA-Lys;
or
(SEQ ID No: 43)
Ala-Lys-Pro-Ser-Tyr-Hyp-Hyp-Thr-DOPA-Lys-Ala-
Lys-Pro-Ser-Tyr-Hyp-Hyp-Thr-DOPA-Lys-Ala-Lys-
Pro-Ser-Tyr-Hyp-Hyp-Thr-DOPA-Lys-Ala-Lys-Pro-
Ser-Tyr-Hyp-Hyp-Thr-DOPA-Lys-Ala-Lys-Pro-Ser-
Tyr-Hyp-Hyp-Thr-DOPA-Lys.

29-30. (canceled)

31. A pharmaceutical formulation comprising the compound as defined in claim 1.

32. The pharmaceutical formulation as claimed in claim 31, which further comprises a pharmaceutically- or cosmetically-acceptable, adjuvant, diluent or carrier.

33. The pharmaceutical formulation as claimed in claim 31 that is suitable for, adapted for, and/or packaged and presented for, topical administration, wherein the pharmaceutically- or cosmetically-acceptable adjuvant, diluent or carrier is a topical adjuvant, diluent or carrier.

34. The pharmaceutical formulation as claimed in claim 31, which is in the form of a gel, a spray, a cream, an ointment or a dry powder.

35. The pharmaceutical formulation as claimed in claim 31, which further includes a further pharmaceutically-active ingredient.

36. A kit of parts comprising components:
(A) the compound as defined in claim 1 or a pharmaceutical formulation that comprises the compound; and
(B) a pharmaceutical formulation including a further pharmaceutically-active ingredient in admixture with a pharmaceutically-acceptable adjuvant, diluent or carrier, which components (A) and (B) are each provided in a form that is suitable for administration in conjunction with the other.

37. The pharmaceutical formulation as claimed in claim 35 wherein the further pharmaceutically-active ingredient is an anti-inflammatory agent, a pro-inflammatory agent, an antibiotic, an anti-bacterial and/or antiprotozoal agent, an antiviral agent, an anaesthetic and/or a wound recovery drug.

38. The pharmaceutical formulation as claimed in claim 37, wherein the pharmaceutically-active ingredient is an antiinflammatory agent.

39-40. (canceled)

41. A method of treatment of inflammation, an inflammatory disorder, and/or of a disorder characterised by inflammation, which method comprises the administration of a compound as defined in claim 1 to a patient in need of such treatment.

42. The method as claimed in claim 41, wherein the disorder characterised by inflammation is, or results in, a wound or a burn.

43. The method as claimed in claim 42, wherein the disorder resulting in a wound is haemorrhoids or ulcerative colitis.

44. The method as claimed in claim 42, wherein the compound or salt thereof is administered topically in the form of a topical formulation.

45. The method as claimed in claim 44, wherein said administration is carried out by direct topical administration to skin.

46. The method as claimed in claim 44, wherein said administration is carried out by direct topical administration to a mucosal surface.

47. The method as claimed in claim 41, wherein said administration is carried by oral, intravenous, cutaneous or subcutaneous, nasal, intramuscular, intraperitoneal, pulmonary or anorectal delivery.

48. (canceled)

49. The method as claimed in claim 41, wherein W is HCA, HCA-Ala-, DOPA or DOPA-Ala- and/or U is DOPA, and the compound is crosslinked, before or after said administration to a subject.

50-56. (canceled)


SEQUENCE LISTING (100 sequences)

SEQ ID NO: 1 - protein, 10 aa, Mytilus sp.
  mefp-1 decapeptide
  SITE 6,7: Wherein Xaa is hydroxyproline
  SITE 9: Wherein Xaa is 3,4-dihydroxyphenylalanine
AKPSYXXTXK

SEQ ID NO: 2 - protein, 10 aa, Mytilus sp.
  Mefp-1 decapepdite analogue
  SITE 6,7: Wherein Xaa is hydroxyproline
AKPSYXXTYK

SEQ ID NO: 3 - protein, 24 aa, Artificial Sequence
  REPEAT 1..12: Wherein the sequence features 1 to 4 times, or is absent
  Peptide component
  VARIANT 1,13: Wherein Xaa is selected from the group lysine, alanine, 3,4-dihydroxyphenylalanine and a 3,4-dihydrocinnamic acid (HCA)residue, provided that, when present, the HCA residue is located at the N-terminus of the peptide sequence
  VARIANT 2,14: Wherein Xaa is selected from the group lysine, alanine, and 3,4-dihydroxyphenylalanine, or is absent
  VARIANT 4,16: Wherein Xaa represents proline, hydroxyproline, or dihydroxyproline
  VARIANT 6,18: Wherein Xaa represents tyrosine or 3,4- dihydroxyphenylalanine
  VARIANT 7,19: Wherein Xaa represents serine, proline, hydroxyproline, or dihydroxyproline
  VARIANT 8,20: Wherein Xaa is selected from the group lysine, alanine, proline, hydroxyproline, dihydroxyproline, threonine, 3,4-dihydroxyphenylalanine and tyrosine
  VARIANT 9,10,11,12,21,22,23,24: Wherein Xaa is selected from the group lysine, alanine, proline,hydroxyproline, dihydroxyproline, threonine, 3,4-dihydroxyphenylalanine and tyrosine, or is absent
XXKXSXXXXXXXXXKXSXXXXXXX

SEQ ID NO: 4 - protein, 10 aa, Artificial Sequence
  Peptide component
  SITE 6,9: Wherein Xaa is 3,4-dihydroxyphenylalanine
AKPSYXTYXK

SEQ ID NO: 5 - protein, 10 aa, Artificial Sequence
  Peptide component
  SITE 6,9: Wherein Xaa is hydroxyproline
  SITE 8: Wherein Xaa is 3,4-dihydroxyphenylalanine
AKPSYXTXXK

SEQ ID NO: 6 - protein, 11 aa, Artificial Sequence
  Peptide component
  SITE 6,9: Wherein Xaa is hydroxyproline
  SITE 11: Wherein Xaa is 3,4-dihydroxyphenylalanine
AKPSYXTYXKX

SEQ ID NO: 7 - protein, 20 aa, Artificial Sequence
  Peptide component
  SITE 7,17: Wherein Xaa is hydroxyproline
AKPSYSXTYKAKPSYSXTYK

SEQ ID NO: 8 - protein, 50 aa, Artificial Sequence
  Peptide component
  SITE 6,7,16,17,26,27,36,37,46,47: Wherein Xaa is hydroxyproline
AKPSYXXTYKAKPSYXXTYKAKPSYXXTYKAKPSYXXTYKAKPSYXXTYK

SEQ ID NO: 9 - protein, 10 aa, Artificial Sequence
  Peptide component
  SITE 7,8: Wherein Xaa is hydroxyproline
KAKPSYXXTY

SEQ ID NO: 10 - protein, 10 aa, Artificial Sequence
  Peptide component
  SITE 4,7,8: Wherein Xaa is hydroxyproline
  SITE 10: Wherein Xaa is 3,4-dihydroxyphenylalanine
KAKXSYXXTX

SEQ ID NO: 11 - protein, 10 aa, Artificial Sequence
  Peptide component
  SITE 7,8: Wherein Xaa is hydroxyproline
  SITE 10: Wherein Xaa is 3,4-dihydroxyphenylalanine
KAKPSYXXTX

SEQ ID NO: 12 - protein, 10 aa, Artificial Sequence
  SITE 1: Wherein Ala is N-terminally modified with a 3,4-dihydrocinnamic acid residue
  Peptide component
  SITE 6,9: Wherein Xaa is hydroxyproline
AKPSYXTYXK

SEQ ID NO: 13 - protein, 10 aa, Artificial Sequence
  SITE 1: Wherein Ala is N-terminally modified with a 3,4-dihydrocinnamic acid residue
  Peptide component
  SITE 6,7: Wherein Xaa is hydroxyproline
AKPSYXXTYK

SEQ ID NO: 14 - protein, 9 aa, Artificial Sequence
  SITE 1: Wherein Lys is N-terminally modified with a 3,4-dihydrocinnamic acid residue
  Peptide component
  SITE 5,6: Wherein Xaa is hydroxyproline
KPSYXXTAK

SEQ ID NO: 15 - protein, 9 aa, Artificial Sequence
  SITE 1: Wherein Lys is N-terminally modified with a 3,4-dihydrocinnamic acid residue
  Peptide component
  SITE 5,8: Wherein Xaa is hydroxyproline
KPSYXTAXK

SEQ ID NO: 16 - protein, 10 aa, Artificial Sequence
  SITE 1: Wherein Xaa is 3,4-dihydroxyphenylalanine
  Peptide component
  SITE 6,9: Wherein Xaa is hydroxyproline
XKPSYXTAXK

SEQ ID NO: 17 - protein, 10 aa, Artificial Sequence
  SITE 1: Wherein Xaa is 3,4-dihydroxyphenylalanine
  Peptide component
  SITE 6,7: Wherein Xaa is hydroxyproline
XKPSYXXTAK

SEQ ID NO: 18 - protein, 11 aa, Artificial Sequence
  SITE 1: Wherein Xaa is 3,4-dihydroxyphenylalanine
  Peptide component
  SITE 7,10: Wherein Xaa is hydroxyproline
XAKPSYXTYXK

SEQ ID NO: 19 - protein, 11 aa, Artificial Sequence
  SITE 1: Wherein Xaa is 3,4-dihydroxyphenylalanine
  Peptide component
  SITE 7,8: Wherein Xaa is hydroxyproline
XAKPSYXXTYK

SEQ ID NO: 20 - protein, 10 aa, Artificial Sequence
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 21 - protein, 10 aa, Artificial Sequence
  Peptide component
  SITE 5,9: Wherein Xaa is 3,4-dihydroxyphenylalanine
  SITE 6,7: Wherein Xaa is hydroxyproline
AKPSXXXTXK

SEQ ID NO: 22 - protein, 10 aa, Artificial Sequence
  Peptide component
  SITE 6,10: Wherein Xaa is 3,4-dihydroxyphenylalanine
  SITE 7,8: Wherein Xaa is hydroxyproline
KAKPSXXXTX

SEQ ID NO: 23 - protein, 10 aa, Artificial Sequence
  Peptide component
  SITE 4,7,8: Wherein Xaa is hydroxyproline
  SITE 6,10: Wherein Xaa is 3,4-dihydroxyphenylalanine
KAKXSXXXTX

SEQ ID NO: 24 - protein, 20 aa, Artificial Sequence
  Peptide component
  SITE 5,9,15,19: Wherein Xaa is 3,4-dihydroxyphenylalanine
  SITE 6,7,16,17: Wherein Xaa is hydroxyproline
AKPSXXXTXKAKPSXXXTXK

SEQ ID NO: 25 - protein, 50 aa, Artificial Sequence
  Peptide component
  SITE 5,9,15,19,25,29,35,39,45,49: Wherein Xaa is 3,4-dihydroxyphenylalanine
  SITE 6,7,16,17,26,27,36,37,46,47: Wherein Xaa is hydroxyproline
AKPSXXXTXKAKPSXXXTXKAKPSXXXTXKAKPSXXXTXKAKPSXXXTXK

SEQ ID NO: 26 - protein, 10 aa, Artificial Sequence
  SITE 1: Wherein Ala is N-terminally modified with a 3,4-dihydrocinnamic acid residue
  Peptide component
  SITE 5,8: Wherein Xaa is 3,4-dihydroxyphenylalanine
  SITE 6,9: Wherein Xaa is hydroxyproline
AKPSXXTXXK

SEQ ID NO: 27 - protein, 10 aa, Artificial Sequence
  SITE 1: Wherein Ala is N-terminally modified with a 3,4-dihydrocinnamic acid residue
  Peptide component
  SITE 5,9: Wherein Xaa is 3,4-dihydroxyphenylalanine
  SITE 6,7: Wherein Xaa is hydroxyproline
AKPSXXXTXK

SEQ ID NO: 28 - protein, 9 aa, Artificial Sequence
  SITE 1: Wherein Lys is N-terminally modified with a 3,4-dihydrocinnamic acid residue
  Peptide component
  SITE 4: Wherein Xaa is 3,4-dihydroxyphenylalanine
  SITE 5,8: Wherein Xaa is hydroxyproline
KPSXXTAXK

SEQ ID NO: 29 - protein, 9 aa, Artificial Sequence
  SITE 1: Wherein Lys is N-terminally modified with a 3,4-dihydrocinnamic acid residue
  Peptide component
  SITE 4: Wherein Xaa is 3,4-dihydroxyphenylalanine
  SITE 5,6: Wherein Xaa is hydroxyproline
KPSXXXTAK

SEQ ID NO: 30 - protein, 10 aa, Artificial Sequence
  SITE 1,5: Wherein Xaa is 3,4-dihydroxyphenylalanine
  Peptide component
  SITE 6,9: Wherein Xaa is hydroxyproline
XKPSXXTAXK

SEQ ID NO: 31 - protein, 10 aa, Artificial Sequence
  SITE 1,5: Wherein Xaa is 3,4-dihydroxyphenylalanine
  Peptide component
  SITE 6,7: Wherein Xaa is hydroxyproline
XKPSXXXTAK

SEQ ID NO: 32 - protein, 11 aa, Artificial Sequence
  SITE 1,6,9: Wherein Xaa is 3,4-dihydroxyphenylalanine
  Peptide component
  SITE 7,10: Wherein Xaa is hydroxyproline
XAKPSXXTXXK

SEQ ID NO: 33 - protein, 11 aa, Artificial Sequence
  SITE 1,6: Wherein Xaa is 3,4-dihydroxyphenylalanine
  Peptide component
  SITE 7,8: Wherein Xaa is hydroxyproline
XAKPSXXXTYK

SEQ ID NO: 34 - protein, 11 aa, Artificial Sequence
  SITE 1,6,10: Wherein Xaa is 3,4-dihydroxyphenylalanine
  Peptide component
  SITE 7,8: Wherein Xaa is hydroxyproline
XAKPSXXXTXK

SEQ ID NO: 35 - protein, 23 aa, Artificial Sequence
  REPEAT 1..11: Wherein the sequence features 1 to 4 times
  Peptide component
  VARIANT 3,14: Wherein Xaa represents proline, hydroxyproline, or dihydroxyproline
  VARIANT 5,16: Wherein Xaa represents tyrosine or 3,4-dihydroxyphenylalanine
  VARIANT 6,17: Wherein Xaa represents serine, proline, hydroxyproline, or dihydroxyproline
  SITE 7,18: Wherein Xaa is selected from the group lysine, alanine, proline,hydroxyproline, dihydroxyproline, threonine, 3,4-dihydroxyphenylalanine and tyrosine
  VARIANT 8,9,10,11,19,20,21,22: Wherein Xaa is selected from the group lysine, alanine, proline,hydroxyproline, dihydroxyproline, threonine, 3,4-dihydroxyphenylalanine and tyrosine, or is absent
  SITE 23: Wherein Xaa represents 3,4- dihydroxyphenylalanine or dopamine or a dopamine fragment, or is absent
AKXSXXXXXXXAKXSXXXXXXXX

SEQ ID NO: 36 - protein, 20 aa, Artificial Sequence
  Peptide component
  SITE 7,17: Wherein Xaa is hydroxyproline
AKPSYSXTYKAKPSYSXTYK

SEQ ID NO: 37 - protein, 50 aa, Artificial Sequence; identical to SEQ ID NO: 8
  Peptide component
  SITE 6,7,16,17,26,27,36,37,46,47: Wherein Xaa is hydroxyproline

SEQ ID NO: 38 - protein, 20 aa, Artificial Sequence
  Peptide component
  SITE 5,9,15,19: Wherein Xaa is 3,4-dihydroxyphenylalanine
  SITE 6,7,16,17: Wherein Xaa is hydroxyproline
AKPSXXXTXKAKPSXXXTXK

SEQ ID NO: 39 - protein, 50 aa, Artificial Sequence; identical to SEQ ID NO: 25
  Peptide component
  SITE 5,9,15,19,25,29,35,39,45,49: Wherein Xaa is 3,4-dihydroxyphenylalanine
  SITE 6,7,16,17,26,27,36,37,46,47: Wherein Xaa is hydroxyproline

SEQ ID NO: 40 - protein, 20 aa, Artificial Sequence
  Peptide component
  SITE 6,7,16,17: Wherein Xaa is hydroxyproline
  SITE 9,19: Wherein Xaa is 3,4-dihydroxyphenylalanine
AKPSYXXTXKAKPSYXXTXK

SEQ ID NO: 41 - protein, 30 aa, Artificial Sequence
  Peptide component
  SITE 6,7,16,17,26,27: Wherein Xaa is hydroxyproline
  SITE 9,19,29: Wherein Xaa is 3,4-dihydroxyphenylalanine
AKPSYXXTXKAKPSYXXTXKAKPSYXXTXK

SEQ ID NO: 42 - protein, 40 aa, Artificial Sequence
  Peptide component
  SITE 6,7,16,17,26,27,36,37: Wherein Xaa is hydroxyproline
  SITE 9,19,29,39: Wherein Xaa is 3,4-dihydroxyphenylalanine
AKPSYXXTXKAKPSYXXTXKAKPSYXXTXKAKPSYXXTXK

SEQ ID NO: 43 - protein, 50 aa, Artificial Sequence
  Peptide component
  SITE 6,7,16,17,26,27,36,37,46,47: Wherein Xaa is hydroxyproline
  SITE 9,19,29,39,49: Wherein Xaa is 3,4-dihydroxyphenylalanine
AKPSYXXTXKAKPSYXXTXKAKPSYXXTXKAKPSYXXTXKAKPSYXXTXK

SEQ ID NO: 44 - skipped

SEQ ID NO: 45 - protein, 11 aa, Artificial Sequence
  REPEAT 1..10: Wherein the sequence features 2 times
  Peptide component
  SITE 6,7: Wherein Xaa is hydroxyproline
  SITE 9: Wherein Xaa is 3,4-dihydroxyphenylalanine
AKPSYXXTXKK

SEQ ID NO: 46 - protein, 11 aa, Artificial Sequence
  REPEAT 1..10: Wherien the sequence features 2 times
  Peptide component
  SITE 6,7: Wherein Xaa is hydroxyproline
  SITE 9: Wherein Xaa is 3,4-dihydroxyphenylalanine
  SITE 11: Wherein Xaa is Ornithine
AKPSYXXTXKX

SEQ ID NO: 47 - protein, 11 aa, Artificial Sequence
  REPEAT 1..10: Wherein the sequence features 2 times
  Peptide component
  SITE 6,7: Wherein Xaa is hydroxyproline
  SITE 9: Wherein Xaa is 3,4-dihydroxyphenylalanine
  SITE 11: Wherein Xaa is diaminopropionic acid
AKPSYXXTXKX

SEQ ID NO: 48 - protein, 12 aa, Artificial Sequence
  REPEAT 1..10: Wherein the sequence features 2 times
  REPEAT 1..11: Wherein the sequence features 2 times
  Peptide component
  SITE 6,7: Wherein Xaa is hydroxyproline
  SITE 9: Wherein Xaa is 3,4-dihydroxyphenylalanine
AKPSYXXTXKKK

SEQ ID NO: 49 - protein, 12 aa, Artificial Sequence
  REPEAT 1..10: Wherein the sequence features 2 times
  REPEAT 1..11: Wherein the sequence features 2 times
  Peptide component
  SITE 6,7: Wherein Xaa is hydroxyproline
  SITE 9: Wherein Xaa is 3,4-dihydroxyphenylalanine
  SITE 11,12: Wherein Xaa is Ornithine
AKPSYXXTXKXX

SEQ ID NO: 50 - protein, 12 aa, Artificial Sequence
  REPEAT 1..10: Wherein the sequence features 2 times
  REPEAT 1..11: Wherein the sequence features 2 times
  Peptide component
  SITE 6,7: Wherein Xaa is hydroxyproline
  SITE 9: Wherein Xaa is 3,4-dihydroxyphenylalanine
  SITE 11,12: Wherein Xaa is diaminopropionic acid
AKPSYXXTXKXX

SEQ ID NO: 51 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,7: Wherein Xaa is hydroxyproline
  SITE 9: Wherein Xaa is 3,4-dihydroxyphenylalanine
AKPSYXXTXKKKK

SEQ ID NO: 52 - protein, 14 aa, Artificial Sequence
  SITE 1: Wherein Xaa is 3,4-dihydroxyphenylalanine
  REPEAT 1..11: Wherein the sequence features 2 times
  REPEAT 1..12: Wherein the sequence features 2 times
  REPEAT 1..13: Wherein the sequence features 2 times
  Peptide component
  SITE 7,8: Wherein Xaa is hydroxyproline
XAKPSYXXTYKKKK

SEQ ID NO: 53 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,7: Wherein Xaa is hydroxyproline
  SITE 9: Wherein Xaa is 3,4-dihydroxyphenylalanine
  SITE 11,12,13: Wherein Xaa is Ornithine
AKPSYXXTXKXXX

SEQ ID NO: 54 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,7: Wherein Xaa is hydroxyproline
  SITE 9: Wherein Xaa is 3,4-dihydroxyphenylalanine
  SITE 11,12,13: Wherein Xaa is diaminopropionic acid
AKPSYXXTXKXXX

SEQ ID NO: 55 - protein, 11 aa, Artificial Sequence
  REPEAT 1..10: Wherein the sequence features 2 times
  Peptide component
  SITE 6,7: Wherein Xaa is hydroxyproline
AKPSYXXTYKK

SEQ ID NO: 56 - protein, 11 aa, Artificial Sequence
  REPEAT 1..10: Wherein the sequence features 2 times
  Peptide component
  SITE 6,9: Wherein Xaa is hydroxyproline
AKPSYXTYXKK

SEQ ID NO: 57 - protein, 11 aa, Artificial Sequence
  REPEAT 1..10: Wherein the sequence features 2 times
  Peptide component
  SITE 6,9: Wherein Xaa is hydroxyproline
  SITE 8: Wherein Xaa is 3,4-dihydroxyphenylalanine
AKPSYXTXXKK

SEQ ID NO: 58 - protein, 12 aa, Artificial Sequence
  SITE 1: Wherein Xaa is 3,4-dihydroxyphenylalanine
  REPEAT 1..11: Wherein the sequence features 2 times
  Peptide component
  SITE 7,10: Wherein Xaa is hydroxyproline
XAKPSYXTYXKK

SEQ ID NO: 59 - protein, 11 aa, Artificial Sequence
  REPEAT 1..10: Wherein the sequence features 2 times
  Peptide component
  SITE 5,8: Wherein Xaa is 3,4-dihydroxyphenylalanine
  SITE 6,9: Wherein Xaa is hydroxyproline
AKPSXXTXXKK

SEQ ID NO: 60 - protein, 12 aa, Artificial Sequence
  SITE 1,6,9: Wherein Xaa is 3,4-dihydroxyphenylalanine
  REPEAT 1..11: Wherein the sequence features 2 times
  Peptide component
  SITE 7,10: Wherein Xaa is hydroxyproline
XAKPSXXTXXKK

SEQ ID NO: 61 - protein, 12 aa, Artificial Sequence
  REPEAT 1..10: Wherein the sequence features 2 times
  REPEAT 1..11: Wherein the sequence features 2 times
  Peptide component
  SITE 6,7: Wherein Xaa is hydroxyproline
AKPSYXXTYKKK

SEQ ID NO: 62 - protein, 12 aa, Artificial Sequence
  REPEAT 1..10: Wherein the sequence features 2 times
  REGION 1..11: Wherein the sequence features 2 times
  Peptide component
  SITE 6,9: Wherein Xaa is hydroxyproline
AKPSYXTYXKKK

SEQ ID NO: 63 - protein, 12 aa, Artificial Sequence
  REPEAT 1..10: Wherein the sequence features 2 times
  REPEAT 1..11: Wherein the sequence features 2 times
  Peptide component
  SITE 6,9: Wherein Xaa is hydroxyproline
  SITE 8: Wherein Xaa is 3,4-dihydroxyphenylalanine
AKPSYXTXXKKK

SEQ ID NO: 64 - protein, 13 aa, Artificial Sequence
  SITE 1: Wherein Xaa is 3,4-dihydroxyphenylalanine
  REPEAT 1..11: Wherein the sequence features 2 times
  REPEAT 1..12: Wherein the sequence features 2 times
  Peptide component
  SITE 7,10: Wherein Xaa is hydroxyproline
XAKPSYXTYXKKK

SEQ ID NO: 65 - protein, 12 aa, Artificial Sequence
  REPEAT 1..10: Wherein the sequence features 2 times
  REPEAT 1..11: Wherein the sequence features 2 times
  Peptide component
  SITE 5,8: Wherein Xaa is 3,4-dihydroxyphenylalanine
  SITE 6,9: Wherein Xaa is hydroxyproline
AKPSXXTXXKKK

SEQ ID NO: 66 - protein, 13 aa, Artificial Sequence
  SITE 1,6,9: Wherein Xaa is 3,4-dihydroxyphenylalanine
  REPEAT 1..11: Wherein the sequence features 2 times
  REPEAT 1..12: Wherein the sequence features 2 times
  Peptide component
  SITE 7,10: Wherein Xaa is hydroxyproline
XAKPSXXTXXKKK

SEQ ID NO: 67 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,7: Wherein Xaa is hydroxyproline
AKPSYXXTYKKKK

SEQ ID NO: 68 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,9: Wherein Xaa is hydroxyproline
AKPSYXTYXKKKK

SEQ ID NO: 69 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,9: Wherein Xaa is hydroxyproline
  SITE 8: Wherein Xaa is 3,4-dihydroxyphenylalanine
AKPSYXTXXKKKK

SEQ ID NO: 70 - protein, 14 aa, Artificial Sequence
  SITE 1: Wherein Xaa is 3,4-dihydroxyphenylalanine
  REPEAT 1..11: Wherein the sequence features 2 times
  REPEAT 1..12: Wherein the sequence features 2 times
  REPEAT 1..13: Wherein the sequence features 2 times
  Peptide component
  SITE 7,10: Wherein Xaa is hydroxyproline
XAKPSYXTYXKKKK

SEQ ID NO: 71 - protein, 13 aa, Artificial Sequence
  REPEAT 1..10: Wherein the sequence features 2 times
  REPEAT 1..11: Wherein the sequence features 2 times
  REPEAT 1..12: Wherein the sequence features 2 times
  Peptide component
  SITE 5,8: Wherein Xaa is 3,4-dihydroxyphenylalanine
  SITE 6,9: Wherein Xaa is hydroxyproline
AKPSXXTXXKKKK

SEQ ID NO: 72 - protein, 14 aa, Artificial Sequence
  SITE 1,6,9: Wherein Xaa is 3,4-dihydroxyphenylalanine
  REPEAT 1..11: Wherein the sequence features 2 times
  REPEAT 1..12: Wherein the sequence features 2 times
  REPEAT 1..13: Wherein the sequence features 2 times
  Peptide component
  SITE 7,10: Wherein Xaa is hydroxyproline
XAKPSXXTXXKKKK

SEQ ID NO: 73 - protein, 11 aa, Artificial Sequence
  SITE 1: Wherein Ala is N terminally modified with a 3,4-dihydrocinnamic acid residue
  REPEAT 1..10: Wherein the sequence features 2 times
  Peptide component
  SITE 6,9: Wherein Xaa is hydroxyproline
AKPSYXTYXKK

SEQ ID NO: 74 - protein, 12 aa, Artificial Sequence
  SITE 1: Wherein Ala is N terminally modified with a 3,4-dihydrocinnamic acid residue
  REPEAT 1..10: Wherein the sequence features 2 times
  REPEAT 1..11: Wherein the sequence features 2 times
  Peptide component
  SITE 6,9: Wherein Xaa is hydroxyproline
AKPSYXTYXKKK

SEQ ID NO: 75 - protein, 13 aa, Artificial Sequence
  SITE 1: Wherein Ala is N terminally modified with a 3,4-dihydrocinnamic acid residue
  REPEAT 1..10: Wherein the sequence features 2 times
  REPEAT 1..11: Wherein the sequence features 2 times
  REPEAT 1..12: Wherein the sequence features 2 times
  Peptide component
  SITE 6,9: Wherein Xaa is hydroxyproline
AKPSYXTYXKKKK

SEQ ID NO: 76 - protein, 12 aa, Artificial Sequence
  REPEAT 1..10: Wherein the sequence features 2 times
  REPEAT 1..11: Wherein the sequence features 2 times
  Peptide component
  SITE 5,9: Wherein Xaa is 3,4-dihydroxyphenylalanine
  SITE 6,7: Wherein Xaa is hydroxyproline
AKPSXXXTXKKK

SEQ ID NO: 77 - protein, 12 aa, Artificial Sequence
  SITE 1: Wherein Ala is N terminally modified with a 3,4-dihydrocinnamic acid residue
  REPEAT 1..10: Wherein the sequence features 2 times
  REPEAT 1..11: Wherein the sequence features 2 times
  Peptide component
  SITE 6,7: Wherein Xaa is hydroxyproline
AKPSYXXTYKKK

SEQ ID NO: 78 - protein, 11 aa, Artificial Sequence
  SITE 1: Wherein Ala is N terminally modified with a 3,4-dihydrocinnamic acid residue
  REPEAT 1..10: Wherein the sequence features 2 times
  Peptide component
  SITE 5,8: Wherein Xaa is 3,4-dihydroxyphenylalanine
  SITE 6,9: Wherein Xaa is hydroxyproline
AKPSXXTXXKK

SEQ ID NO: 79 - protein, 12 aa, Artificial Sequence
  SITE 1,5: Wherein Xaa is 3,4-dihydroxyphenylalanine
  REPEAT 1..10: Wherein the sequence features 2 times
  REPEAT 1..11: Wherein the sequence features 2 times
  Peptide component
  SITE 6,9: Wherein Xaa is hydroxyproline
XKPSXXTAXKKK

SEQ ID NO: 80 - protein, 11 aa, Artificial Sequence
  SITE 1,5: Wherein Xaa is 3,4-dihydroxyphenylalanine
  REPEAT 1..10: Wherein the sequence features 2 times
  Peptide component
  SITE 6,7: Wherein Xaa is hydroxyproline
XKPSXXXTAKK

SEQ ID NO: 81 - protein, 10 aa, Artificial Sequence
  SITE 1: Wherein Lys is N terminally modified with a 3,4-dihydrocinnamic acid residue
  REPEAT 1..9: Wherein the sequence features 2 times
  Peptide component
  SITE 5,6: Wherein Xaa is hydroxyproline
KPSYXXTAKK

SEQ ID NO: 82 - protein, 11 aa, Artificial Sequence
  SITE 1: Wherein Lys is N terminally modified with a 3,4-dihydrocinnamic acid residue
  REPEAT 1..9: Wherein the sequence features 2 times
  REPEAT 1..10: Wherein the sequence features 2 times
  Peptide component
  SITE 4: Wherein Xaa is 3,4-dihydroxyphenylalanine
  SITE 5,8: Wherein Xaa is hydroxyproline
KPSXXTAXKKK

SEQ ID NO: 83 - protein, 12 aa, Artificial Sequence
  REPEAT 1..10: Wherein the sequence features 2 times
  REPEAT 1..11: Wherein the sequence features 2 times
  Peptide component
  SITE 7,8: Wherein Xaa is hydroxyproline
KAKPSYXXTYKK

SEQ ID NO: 84 - protein, 11 aa, Artificial Sequence
  REPEAT 1..10: Wherein the sequence features 2 times
  Peptide component
  SITE 6,10: Wherein Xaa is 3,4-dihydroxyphenylalanine
  SITE 7,8: Wherein Xaa is hydroxyproline
KAKPSXXXTXK

SEQ ID NO: 85 - protein, 12 aa, Artificial Sequence
  REPEAT 1..10: Wherein the sequence features 2 times
  REPEAT 1..11: Wherein the sequence features 2 times
  Peptide component
  SITE 4,7,8: Wherein Xaa is hydroxyproline
  SITE 6,10: Wherein Xaa is 3,4-dihydroxyphenylalanine
KAKXSXXXTXKK

SEQ ID NO: 86 - protein, 4 aa, Artificial Sequence
  Sequence defined by Y
  VARIANT 2,3: Wherein Xaa is selected from the group proline, alanine,hydroxyproline, threonine, 3,4-dihydroxyphenylalanine, and tyrosine
PXXK

SEQ ID NO: 87 - protein, 4 aa, Artificial Sequence
  SITE 1: Wherein Xaa is hydroxyproline
  Sequence defined by Y
  VARIANT 2,3: Wherein Xaa is selected from the group proline, alanine,hydroxyproline, threonine, 3,4-dihydroxyphenylalanine, and tyrosine
XXXK

SEQ ID NO: 88 - protein, 4 aa, Artificial Sequence
  Sequence defined by Y
  VARIANT 2,3: Wherein Xaa is selected from the group proline, alanine,hydroxyproline, threonine, 3,4-dihydroxyphenylalanine, and tyrosine
TXXK

SEQ ID NO: 89 - protein, 4 aa, Artificial Sequence
  Sequence defined by Y
  SITE 3: Wherein Xaa is 3,4-dihydroxyphenylalanine
PTXK

SEQ ID NO: 90 - protein, 4 aa, Artificial Sequence
  Sequence defined by Y
PTYK

SEQ ID NO: 91 - protein, 4 aa, Artificial Sequence
  Sequence defined by Y
TYPK

SEQ ID NO: 92 - protein, 4 aa, Artificial Sequence
  Sequence defined by Y
  SITE 2: Wherein Xaa is 3,4-dihydroxyphenylalanine
TXPK

SEQ ID NO: 93 - protein, 4 aa, Artificial Sequence
  SITE 1: Wherein Xaa is hydroxyproline
  Sequence defined by Y
XTYK

SEQ ID NO: 94 - protein, 4 aa, Artificial Sequence
  SITE 1: Wherein Xaa is hydroxyproline
  Sequence defined by Y
  SITE 3: Wherein Xaa is 3,4-dihydroxyphenylalanine
XTXK

SEQ ID NO: 95 - protein, 4 aa, Artificial Sequence
  SITE 1: Wherein Xaa is hydroxyproline
  Sequence defined by Y
XTAK

SEQ ID NO: 96 - protein, 4 aa, Artificial Sequence
  3
  SITE 3: Wherein Xaa is hydroxyproline
TYXK

SEQ ID NO: 97 - protein, 4 aa, Artificial Sequence
  Sequence defined by Y
  SITE 2: Wherein Xaa is 3,4-dihydroxyphenylalanine
  SITE 3: Wherein Xaa is hydroxyproline
TXXK

SEQ ID NO: 98 - protein, 4 aa, Artificial Sequence
  Sequence defined by Y
  SITE 3: Wherein Xaa is hydroxyproline
TAXK

SEQ ID NO: 99 - protein, 5 aa, Artificial Sequence
  SITE 1,4: Wherein Xaa is hydroxyproline
  Sequence defined by Y
XTYXK

SEQ ID NO: 100 - protein, 5 aa, Artificial Sequence
  Sequence defined by Y
  SITE 3: Wherein Xaa is hydroxyproline
  SITE 5: Wherein Xaa is 3,4-dihydroxyphenylalanine
TYXKX